(19) United States
(12) Patent Application Publication
(10) Pub. No.: US 2025/0114355 A1
(43) Pub. Date: Apr. 10, 2025

(54) TREATING LIVER DISEASE

(71) Applicants: Mayo Foundation for Medical Education and Research, Rochester, MN (US); Regents of the University of Minnesota, Minneapolis, MN (US)

(72) Inventors: Vijay H. Shah, Rochester, MN (US); Sheng Cao, Rochester, MN (US); Mengfei Liu, Rochester, MN (US); Joseph Topczewski, Minneapolis, MN (US); William C.K. Pomerantz, Minneapolis, MN (US); Angela S. Carlson, Minneapolis, MN (US); Huarui Cui, Minneapolis, MN (US); Anand Divakaran, Minneapolis, MN (US)

(21) Appl. No.: 18/289,047

(22) PCT Filed: May 9, 2022
(86) PCT No.: PCT/US2022/028352

§ 371 Date: Oct. 31, 2023

Related U.S. Application Data
(60) Provisional application No. 63/185,895, filed on May 7, 2021.

(51) Int. Cl. A61K 31/506; A61K 31/4545; A61P 1/16

(52) U.S. Cl. CPC A61K 31/506; A61K 31/4545; A61P 1/16

(57) ABSTRACT

This document relates to methods and materials for treating liver diseases (e.g., an alcohol-induced liver disease (ALD) such as alcoholic hepatitis (AH)). For example. one or more inhibitors of a bromodomain-containing protein 4 (BRD4) polypeptide can be administered to a mammal (e.g., a human) having a liver disease (e.g., an ALD such as AH) to treat the mammal.

### CROSS-REFERENCE TO RELATED APPLICATIONS

This application claims the benefit of U.S. Patent Application Ser. No. 63/185,895, filed on May 7, 2021. The disclosure of the prior application is considered part of (and is incorporated by reference in) the disclosure of this application.

### TECHNICAL FIELD

This document relates to methods and materials for treating liver diseases (e.g., an alcohol-induced liver disease (ALD) such as alcoholic hepatitis (AH)). For example, one or more inhibitors of a bromodomain-containing protein 4 (BRD4) polypeptide can be administered to a mammal (e.g., a human) having a liver disease (e.g., ALD such as AH) to treat the mammal.

### BACKGROUND INFORMATION

Alcoholic hepatitis (AH) is a highly morbid condition characterized by acute liver injury in the setting of excess alcohol ingestion. Severe AH can lead to acute-on-chronic liver failure and is associated with a 30-day mortality of greater than 30% with few treatment options (Mathurin et al., J. Hepatol., 36 (4): 480-7 (2002); and Sehrawat et al., Lancet Gastroenterol. Hepatol., 5 (5): 494-506 (2020)).

### SUMMARY

This document provides methods and materials for treating mammals (e.g., humans) having a liver disease (e.g., an ALD such as AH). For example, this document provides inhibitors of a BRD4 polypeptide as well as methods for using inhibitors of a BRD4 polypeptide. In some cases, one or more inhibitors of a BRD4 polypeptide can be administered to a mammal (e.g., a human) having a liver disease to treat the mammal. As demonstrated herein, inhibitors of a BRD4 polypeptide, a transcriptional and epigenetic regulator, can attenuate neutrophil infiltration and liver inflammation associated with AH, and can be used to treat AH.

In general, one aspect of this document features compositions including a compound of Formula (I):

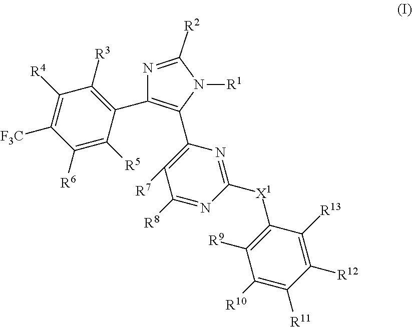

or a pharmaceutically acceptable salt thereof, where R¹ is a 4-7-membered heterocycloalkyl ring comprising at least one N atom, which is optionally substituted with 1, 2, or 3 R^{A} substituents independently selected from C₁₋₆ alkyl, C₂₋₆ alkenyl, C₂₋₆ alkynyl, C₁₋₄ haloalkyl, and C₃₋₅ cycloalkyl, each of which is optionally substituted with 1, 2, or 3 substituents independently selected from OH, NO₂, CN, amino, C₁₋₆ alkylamino, and di(C₁₋₆ alkyl)amino; where X¹ is selected from O and NR^{N}; where R^{N} is selected from H, C₁₋₃ alkyl, and C₁₋₃ haloalkyl; and where each of R², R³, R⁴, R⁵, R⁶, R⁷, R⁸, R⁹, R¹⁰, R¹¹, R¹², and R¹³ is independently selected from H, OH, NO₂, CN, halo, C₁₋₆ alkyl, C₂₋₆ alkenyl, C₂₋₆ alkynyl, C₁₋₄ haloalkyl, C₁₋₆ alkoxy, and C₁₋₆ haloalkoxy, wherein each of said C₁₋₆ alkyl, C₂₋₆ alkenyl, and C₂₋₆ alkynyl is substituted with a substituent independently selected from OH, NO₂, CN, amino, C₁₋₆ alkylamino, and di(C₁₋₆ alkyl)amino. The compound of Formula (I) can have the formula:

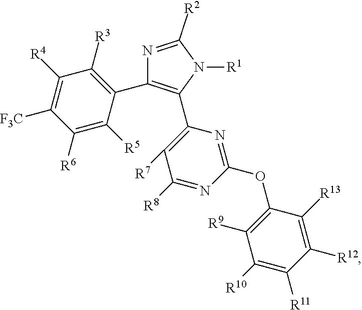

or a pharmaceutically acceptable salt thereof.

The compound of Formula (I) can have the formula:

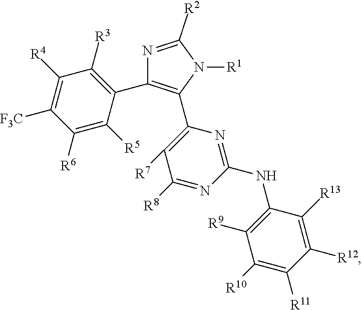

or a pharmaceutically acceptable salt thereof. R¹ can be a 4-7-membered heterocycloalkyl ring comprising at least one N atom, which is optionally substituted with an R^{A} substituent. R¹ can be pyrrolidine or piperidine, each of which is optionally substituted with an R^{A}. R^{A} can be a C₁₋₆ alkyl, substituted with a substituent selected from amino, C₁₋₆ alkylamino, and di(C₁₋₆ alkyl)amino. Each of R², R³, R⁴, R⁵, R⁶, R⁷, R⁸, R⁹, R¹⁰, R¹¹, R¹², and R¹³ can be independently selected from H, halo, and C₁₋₆ alkyl. The compound of Formula (I) can have the formula:

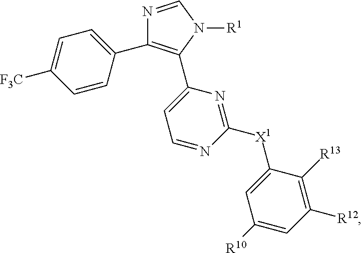

or a pharmaceutically acceptable salt thereof. The compound of Formula (I) can have the formula:

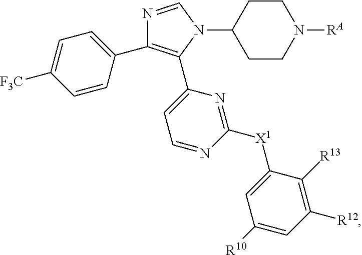

or a pharmaceutically acceptable salt thereof. The compound of Formula (I) can have the formula:

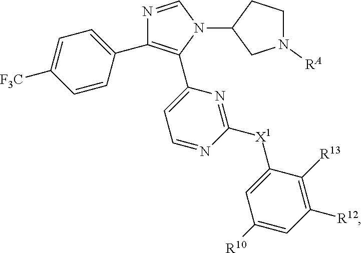

or a pharmaceutically acceptable salt thereof. The compound of Formula (I) can be selected from any one of the following compounds:

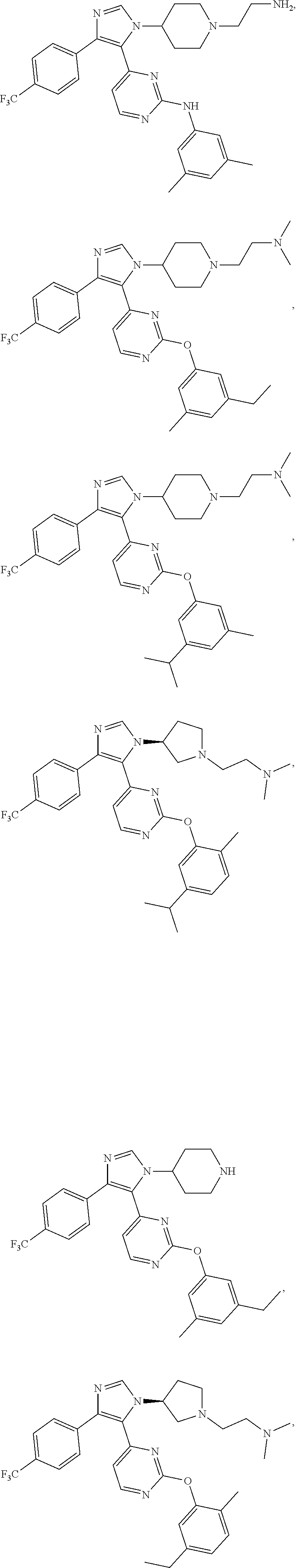

or a pharmaceutically acceptable salt thereof. The compound can inhibit BRD4 polypeptide activity.

In another aspect, this document features compositions including a compound of Formula (II):

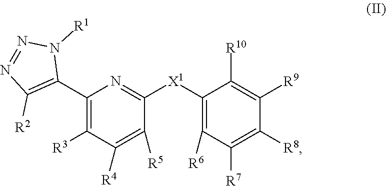

or a pharmaceutically acceptable salt thereof, where R¹ is a 4-7-membered heterocycloalkyl ring comprising at least one N atom, which is optionally substituted with 1, 2, or 3 R^{A} substituents independently selected from C₁₋₆ alkyl, C₂₋₆ alkenyl, C₂₋₆ alkynyl, C₁₋₄ haloalkyl, and C₃₋₅ cycloalkyl, each of which is optionally substituted with 1, 2, or 3 substituents independently selected from OH, NO₂, CN, amino, C₁₋₆ alkylamino, and di(C₁₋₆ alkyl)amino; where X¹ is selected from O and NR^{N}; where R^{N} is selected from H, C₁₋₃ alkyl, and C₁₋₃ haloalkyl; and where each of R², R³, R⁴, R⁵, R⁶, R⁷, R⁸, R⁹, and R¹⁰ is independently selected from H, OH, NO₂, CN, halo, C₁₋₆ alkyl, C₂₋₆ alkenyl, C₂₋₆ alkynyl, C₁₋₄ haloalkyl, C₁₋₆ alkoxy, and C₁₋₆ haloalkoxy, wherein each of said C₁₋₆ alkyl, C₂₋₆ alkenyl, and C₂₋₆ alkynyl is substituted with a substituent independently selected from OH, NO₂, CN, amino, C₁₋₆ alkylamino, and di(C₁₋₆ alkyl)amino. The compound of Formula (II) can have the Formula:

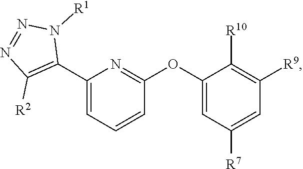

or a pharmaceutically acceptable salt thereof. R¹ can be a 4-7-membered heterocycloalkyl ring comprising at least one N atom, which is optionally substituted with an R^{A} substituent. R^{A} can be a C₁₋₆ alkyl, substituted with a substituent selected from amino, C₁₋₆ alkylamino, and di(C₁₋₆ alkyl)amino. Each of R², R³, R⁴, R⁵, R⁶, R⁷, R⁸, R⁹, and R¹⁰ can be independently selected from H, halo, and C₁₋₆ alkyl. The compound of Formula (II) can have the formula:

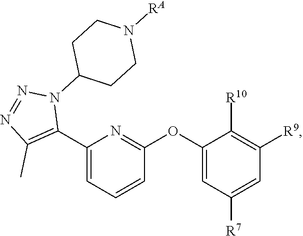

or a pharmaceutically acceptable salt thereof.

The compound of Formula (II) can be

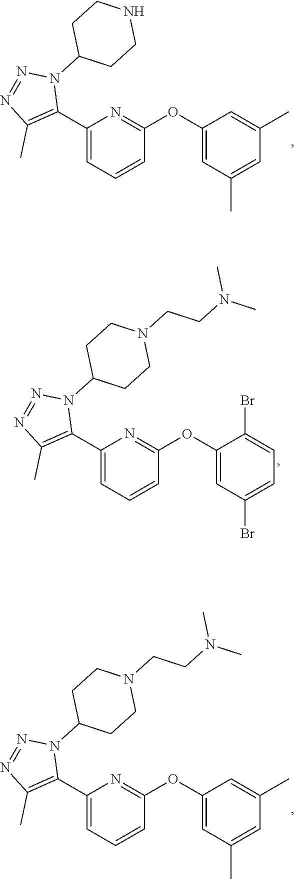 [SMILES: Cc1cc(C)cc(Oc2cccc(-c3c(C)nnn3C3CCNCC3)n2)c1 ; Cc1nnn(C2CCN(CCN(C)C)CC2)c1-c1cccc(Oc2cc(Br)ccc2Br)n1 ; Cc1cc(C)cc(Oc2cccc(-c3c(C)nnn3C3CCN(CCN(C)C)CC3)n2)c1]

or a pharmaceutically acceptable salt thereof. The compound can inhibit BRD4 polypeptide activity.

In another aspect, this document features compositions including a compound of Formula (III):

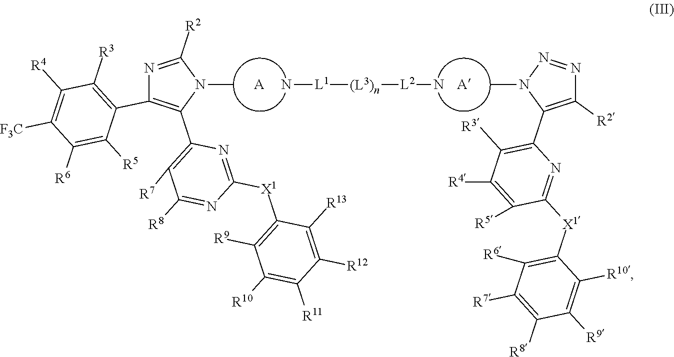

or a pharmaceutically acceptable salt thereof, where each of ring A and ring A′ is independently a 4-7-membered heterocycloalkyl ring comprising at least one N atom, which is optionally substituted with 1, 2, or 3 RA substituents independently selected from C₁₋₆ alkyl, C₂₋₆ alkenyl, C₂₋₆ alkynyl, C₁₋₄ haloalkyl, and C₃₋₅ cycloalkyl, each of which is optionally substituted with 1, 2, or 3 substituents independently selected from OH, NO₂, CN, amino, C₁₋₆ alkylamino, and di(C₁₋₆ alkyl)amino; where each of X¹ and X¹′ is independently selected from O and NR^{N}; where each R^{N} is independently selected from H, C₁₋₃ alkyl, and C₁₋₃ haloalkyl; where each of R², R³, R⁴, R⁵, R⁶, R⁷, R⁸, R⁹, R¹⁰, R¹¹, R¹², R¹³, R^{2′}, R^{3′}, R^{4′}, R^{5′}, R^{6′}, R^{7′}, R^{8′}, R^{9′}, and R^{10′} is independently selected from H, OH, NO₂, CN, halo, C₁₋₆ alkyl, C₂₋₆ alkenyl, C₂₋₆ alkynyl, C₁₋₄ haloalkyl, C₁₋₆ alkoxy, and C₁₋₆ haloalkoxy, wherein each of said C₁₋₆ alkyl, C₂₋₆ alkenyl, and C₂₋₆ alkynyl is substituted with a substituent independently selected from OH, NO₂, CN, amino, C₁₋₆ alkylamino, and di(C₁₋₆alkyl)amino; where each of L¹ and L² is independently C₁₋₃ alkylene, optionally substituted with 1, 2, or 3 substituents independently selected from OH, NO₂, CN, halo, amino, and carboxy; where n is an integer selected from 1 to 10; and where each L³ is independently selected from C(—O), N(R^{N}), O, (—C₁₋₃ alkylene-O—)ₓ, (—O—C₁₋₃ alkylene-)ₓ, and —C₁₋₃ alkylene-, wherein each x is independently an integer from 1 to 10 and each C₁₋₃ alkylene is optionally substituted with 1, 2, or 3 substituents independently selected from OH, NO₂, CN, halo, amino, and carboxy. X¹ can be O and X¹′ can be O. X¹ can be NH and X¹′ can be O. Ring A and ring A′ can each independently be a 4-7-membered heterocycloalkyl ring comprising at least one N atom, which is optionally substituted with an R^{A} substituent. R^{A} can be a C₁₋₆ alkyl, substituted with a substituent selected from amino, C₁₋₆ alkylamino, and di(C₁₋₆ alkyl)amino. Each of R², R³, R⁴, R⁵, R⁶, R⁷, R⁸, R⁹, R¹⁰, R¹¹, R¹², R¹³, R^{2′}, R^{3′}, R^{4′}, R^{5′}, R^{6′}, R^{7′}, R^{8′}, R^{9′}, and R^{10′} can be independently selected from H, halo, and C₁₋₆ alkyl. The compound of Formula (III) can have the formula:

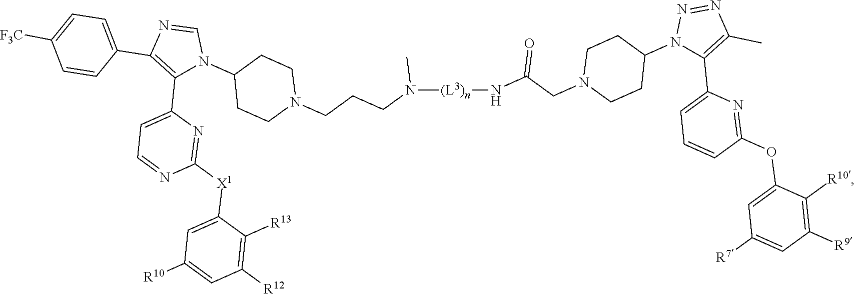

or a pharmaceutically acceptable salt thereof. Each L³ can be selected from —C₁₋₃ alkylene-, (—O—C₁₋₃ alkylene-)ₓ, (—C₁₋₃ alkylene-O—)ₓ, O, NH, C(═O), and N(CH₃). n can be 2, 3, 4, 5, 6, 7, or 8. The compound of Formula (III) can be

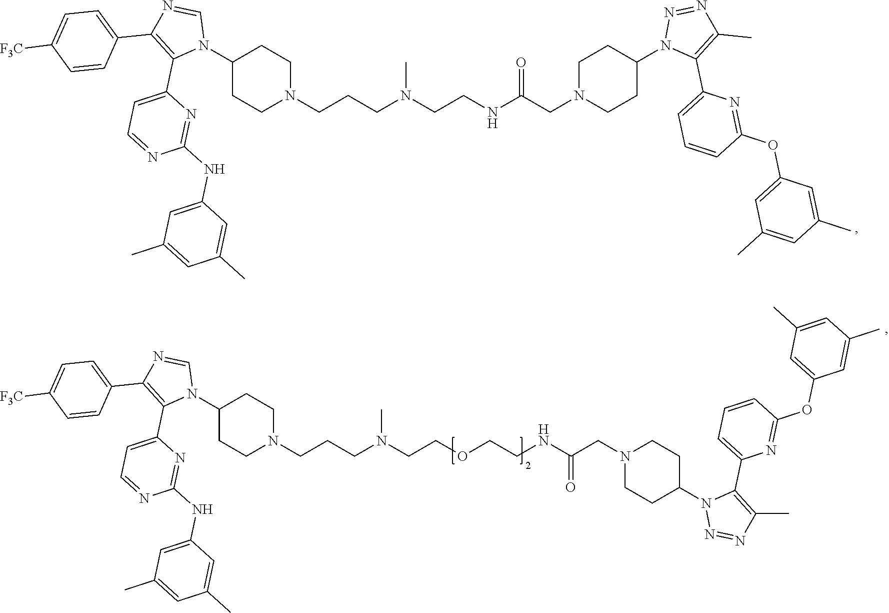

or a pharmaceutically acceptable salt thereof. The compound can inhibit BRD4 polypeptide activity.

In another aspect, this document features methods for inhibiting BRD4 polypeptide activity within a mammal. The methods can include, or consist essentially of, administering a composition including the compound of Formula (I), the compound of Formula (II), and/or the compound of Formula (III) to a mammal. The mammal can be a human. The mammal can have a liver disease. The liver disease can be an ALD. The ALD can be alcoholic hepatitis. The composition can include the compound:

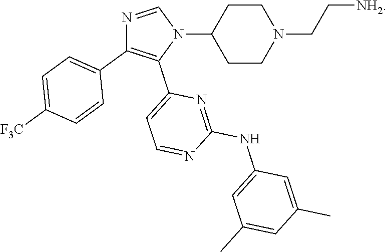 [SMILES: Cc1cc(C)cc(Nc2nccc(-c3c(-c4ccc(C(F)(F)F)cc4)ncn3C3CCN(CCN)CC3)n2)c1]

In another aspect, this document features methods for treating a mammal having a liver disease. The methods can include, or consist essentially of, administering a composition including the compound of Formula (I), the compound of Formula (II), and/or the compound of Formula (III) to a mammal. The mammal can be a human. The liver disease can be an ALD. The ALD can be alcoholic hepatitis. The method can include identifying the mammal as having the liver disease. The method also can include administering an agent used to treat a liver disease to the mammal. The agent can be a nutritional supplement, a corticosteroid, pentoxifylline, an antibiotic, or any combinations thereof. The method also can include subjecting the mammal to a therapy used to treat a liver disease. The therapy can be alcohol cessation counseling or liver transplantation. The composition can include the compound:

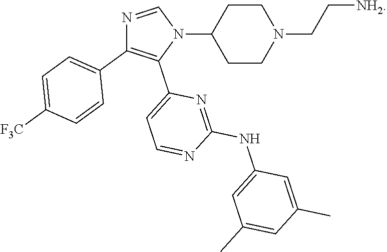 [SMILES: Cc1cc(C)cc(Nc2nccc(-c3c(-c4ccc(C(F)(F)F)cc4)ncn3C3CCN(CCN)CC3)n2)c1]

In another aspect, this document features methods for reducing inflammation in a liver of a mammal having a liver disease. The methods can include, or consist essentially of, administering a composition including the compound of Formula (I), the compound of Formula (II), and/or the compound of Formula (III) to a mammal having a liver disease. The mammal can be a human. The liver disease can be an ALD. The ALD can be alcoholic hepatitis. The composition can include the compound:

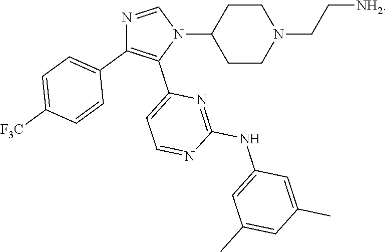 [SMILES: Cc1cc(C)cc(Nc2nccc(-c3c(-c4ccc(C(F)(F)F)cc4)ncn3C3CCN(CCN)CC3)n2)c1]

In another aspect, this document features methods for reducing a number of neutrophils in a liver of a mammal having a liver disease. The methods can include, or consist essentially of, administering a composition including the compound of Formula (I), the compound of Formula (II), and/or the compound of Formula (III) to a mammal having a liver disease. The mammal can be a human. The liver disease can be an ALD. The ALD can be alcoholic hepatitis. The composition can include the compound:

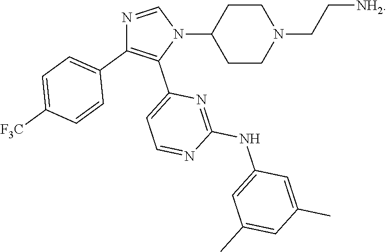 [SMILES: Cc1cc(C)cc(Nc2nccc(-c3c(-c4ccc(C(F)(F)F)cc4)ncn3C3CCN(CCN)CC3)n2)c1]

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIGS. 1A-1F. RNA-Seq and histone mark ChIP-Seq of AH and normal livers show significant differences. FIG. 1A: Schematic of RNA-Seq and ChIP-Seq analyses pipeline. FIG. 1B: Heatmap of differentially expressed genes from the integrated analysis of RNA-Seq and ChIP-Seq. FIG. 1C: Ingenuity pathway analysis (IPA) of differentially upregulated genes from the integrated analysis. Top 10 affected canonical pathways are listed along with their respective inverse log of p-values. FIG. 1D: Differentially expressed genes from the granulocytes/agranulocytes adhesion and diapedesis pathways are listed. Four CXCL chemokines 1, 5, 6, and 8 are located at the same locus and are colored gray. FIG. 1E: Upstream regulator analysis from IPA. Top 10 activated upstream regulators are listed along with their respective normalized z-scores. FIG. 1F: GSEA of TNFα and NF-κB pathway target genes. AH enriched genes are plotted to the left and control enriched genes are plotted to the right. Normalized Enrichment Score (NES) and False Discover Rate (FDR) are listed for the analyses.

FIGS. 2A-2C. LSECs are the major source of CXCL chemokines in the liver under control of TNFα/NF-κB Signaling. FIG. 2A: RNA-Seq of normal liver cell types. Expression levels of CXCL genes (expressed in RPKM) were normalized and ratios were plotted. Error bars indicate SD. One-way ANOVA analysis was performed, with Post-hoc Dunnett's multiple comparison correction (n=3 for each cell type). FIG. 2B: TNFα significantly increased expression of CXCL chemokines revealed by qPCR (n=4) and by ELISA (n=11). CXCL chemokines expression is shown as fold change over control condition for qPCR and as pg/mL by ELISA. Error bars indicate SD. Paired/test analysis was performed on the log-transformed values. FIG. 2C: LSECs were pretreated with varying amounts of celastrol (0 to 5 μM) and exposed to medium with or without TNFα. qPCRs were performed for expression of CXCL 1, 6, and 8, and are shown in log₁₀ fold over basal expression. Error bars indicate SD. One-way matched-pairs ANOVA analysis was performed with Post-hoc Dunnett's multiple comparison correction (n=6). There was a significant linear trend of decreasing CXCL1, 6, and 8 expression with increasing celastrol concentrations with TNFα (p<0.0001 for all groups).

FIGS. 3A-3F. Identification of a super enhancer for CXCL chemokines. FIG. 3A: 4C was performed on LSEC cells with and without TNFα stimulation. Interactions with CXCL1 promoter were plotted using fragment read counts. A genome region of about 75 kb contained two peaks of CXCL1 interaction under TNFα stimulation (boxed). Viewpoint (VP) was labeled with a black line indicating the location of the reference sequence. FIG. 3B: H3K27 acetylation (H3K27ac) and H3K4 methylation (H3K4me1) ChIP-Seq signals of normal (shown in blue) and AH (shown in red) livers were plotted. ⋅ indicates the location of the NF-κB site (E1) targeted for subsequent analyses. Scale bar represents 50 kb. FIG. 3C: ChIP-qPCR assays for BRD4 and NF-κB binding at the aforementioned locus (⋅). Sequence enrichment was normalized to input. Error bar indicates SD. Paired/test analysis was performed on percent input values (n=8). FIG. 3D and FIG. 3E: ROSE algorithm of putative super enhancer analysis from LSECs without (FIG. 3D) or with (FIG. 3E) TNFα treatment. Region contained in the dashed box contained sequences with top H3K27ac enrichment and are considered to be putative super enhancers. FIG. 3F: 3C experiments were performed on LSECs to detect interaction of the predicted CXCL super enhancer with promoters of various CXCLs without (thick line) and with TNFα (thin line). The aforementioned NF-κB binding site (⋅) within the CXCL super-enhancer (dash lines) was used as reference sequence. Interaction frequencies were plotted after being normalized to that of RASSF6, a nearby non-inflammatory gene used as control. Multiple other sequences were selected between target CXCL promoters as additional controls. X-axis maps relevant gene sequences as distance (in kb) from RASSF6. Two-way paired ANOVA analysis was performed on the relative interaction frequencies at target sites followed by Post-hoc Sidak's multiple comparison correction (n=5). Treatment with TNFα was found to be a significant variable (p=0.0034). CXCL1, 2, 3, 6, and 8 loci were found to have increased interaction frequencies with p values as labeled.

FIGS. 4A-4D. Histone modifications at CXCL super enhancer and CXCL promoter sites modulate chemokine gene expression. FIG. 4A: Schematic of dCas9-KRAB binding with sgRNA leading to epigenetic silencing. FIG. 4B: dCas9-KRAB fusion protein targeting the selected NF-κB site within CXCL super-enhancer suppressed CXCL expression in LSECs. An sgRNA with specificity for a predicted NF-κB binding site on the CXCL enhancer decreased CXCL1, 6, and 8 expression revealed by qPCR, but did not affect expression of MTHFD2L, a nearby noninflammatory gene (negative control) (n=8). Changes in chemokine expression were calculated as fold change over basal expression and log₁₀ (fold change) was plotted on the y-axis. ELISA for CXCL1 was performed on supernatants and mirrored the pattern seen from qPCR (n=6). Error bars indicate SD. Two-way matched-pairs ANOVA was performed on log-transformed fold-change values with Post-hoc Tukey's multiple comparison correction. FIG. 4C: Another sgRNA targeting a predicted NF-κB binding site on CXCL1 promoter decreased expression of CXCL1, but not other CXCLs by qPCR (n=8) and CXCL1 ELISA (n=6). Log₁₀ (fold change) was plotted. Error bars indicate SD. Two-way matched-pairs ANOVA was performed on log-transformed fold change values, with Post hoc Tukey's multiple comparison correction. FIG. 4D: ChIP-qPCR for H3K9me3 on dCas9-KRAB treated cells. dCas9-KRAB was co-transduced with sgRNA targeting CXCL promoter (P), CXCL super-enhancer (E), or empty vector (C), and treated with or without TNFα. ChIP-qPCR was performed with anti-H3K9me3 antibody or isotype control. Enrichment for either CXCL1 promoter or CXCL super-enhancer sequence was examined. Y-axis plots percent input (n=3). Error bars indicate SD. Two-way matched-pairs ANOVA was performed with Post-hoc Dunnett's multiple comparison correction.

FIGS. 5A-5C. Bromodomain inhibitors suppress expression of CXCLs by inhibition of transcription factor binding at CXCL super enhancer and promoter sites. FIG. 5A: LSECs were pretreated with UMN627 (0-50 μM) (n=5). CXCL1, 6, and 8 expression levels were assessed by qPCR. Expression levels were normalized to basal condition and log₁₀ fold change values were plotted. One-way matched-pairs ANOVA analysis was performed with Post-hoc Dunnett's multiple comparison correction. FIG. 5B and FIG. 5C: Same experiments were repeated with celastrol. Enrichment for either CXCL1 promoter or CXCL super enhancer sequence was examined. Sequence enrichment was normalized to input. One-way ANOVA analysis was performed with Post-hoc Tukey's multiple comparison correction. Error bars indicate SD. There were significant linear trends of decreasing CXCL1, 6, 8 expression with increasing UMN627 concentration without and with TNF (p<0.0001 for all groups).

FIGS. 6A-6D. Bromodomain inhibitor UMN627 attenuates liver CXCL production and neutrophil infiltration in an alcohol binge/LPS model. FIG. 6A: Schematic of alcohol binge/LPS model protocol. Mice underwent once-daily gastric gavage with alcohol or maltose dextran for 3 days. A subset of mice also received IP injection of UMN627. Alcohol-gavaged mice also received IP injection of LPS on day 4, 8 hours before sacrifice (n=7 Control/Veh, n=10 Binge+LPS/Veh, n=7 Control/UMN627, and n=12 Binge+LPS/UMN627). FIG. 6B: qPCRs demonstrated CXCL chemokine and neutrophil marker Ly6g expression elevation with alcohol/LPS treatment. This response was attenuated by UMN627. Expression levels were normalized to average expression of maltose gavaged control mice. FIG. 6C: IHC for MPO is shown, with number of neutrophils per low power fields on y-axis. FIG. 6D: Serum ALT levels did not show statistical difference among various groups. Two-way ANOVA was performed on normalized expression values for qPCRs or cell counts for IHC staining or ALT values, with Post-hoc Tukey's multiple comparison correction. Error bars indicate SD.

FIG. 7. Clinical Characteristics of AH patients. The median and IQR of various clinical parameters are listed.

FIGS. 8A-8C. Principle component analysis of AH and control liver gene expression profiles demonstrate differential clustering. FIGS. 8A-8C: PCA of all genes from RNA-seq showed clear separation of clinical samples into 2 clusters based on control and AH status. Expression level of differential genes with congruent histone marks from integrated analysis (FIG. 8B) and genes in the Granulocyte Adhesion/Diapedesis pathway (FIG. 8C) were analyzed and showed similar separation.

FIGS. 9A-9B. Heatmap of RNA-seq with histone ChIP-seq profiles. FIG. 9A: Heatmap of RNA-seq and ChIP-seq for marks H3K4me3, H3K4me1, H3K27ac, and H3K27me3 for differentially expressed genes are plotted. ChIP-seq signal over TSS±2kb was estimated as counts per 10M uniquely mapped, non-redundant reads in log2 scaled and quantile normalized. Z-scores were plotted. FIG. 9B: The input-subtracted read density (RPM, reads per million) in 100-bp non-overlapping bins over the TSS±5kb region was plotted separately for the AH up- and down-regulated genes. Bin signal of all protein-coding genes was quantile normalized. There was an increase of signal from AH samples for active marks H3K27ac and H3K4me3 in AH up-regulated genes. Conversely, there was a decrease of signal from AH samples for repressive mark H3K27me3 in AH down-regulated genes.

FIG. 10. Correlation of four histone mark profiles with gene expression change. The 761 genes down regulated in AH were split into three groups based on H3K4me3 status in the proximal regulatory regions: no peaks, no signal change, and reduced signals as identified by DiffBind analysis. For genes in each of the three groups, the number of genes showing the expected changes in histone modifications (i.e., increased signals for H3K27me3 and decreased signals for the other three active marks) in the proximal regulatory regions (TSS±2kb) and H3K27ac in the distal regulatory regions (≥2.5 kb away from TSS) was estimated (FDR≤0.01 and log2 (fold change)≥1 in DiffBind). For the 761 genes down-regulated in AH, 283 genes showed reduced H3K4me3 in the promoters compared to normal. Of the remaining 478 genes whose promoters had no H3K4me3 peaks (184 genes) or no H3K4me3 changes (294 genes), 229 (158+71) were associated with increased H3K27me3 or decreased H3K4me1/H3K27ac in the promoters, or decreased H3K27ac in the nearest enhancers. Thus, two-thirds (512/761) of the AH down-regulated genes appeared to be associated with the expected chromatin changes in the promoters or enhancers. On the other hand, for the 950 genes up-regulated in AH, 54 genes showed increased H3K4me3 in the promoters compared to normal. Of the remaining 896 genes with no H3K4me3 peaks (231) or no H3K4me3 changes (664) in promoters, 342 (281+61) were associated with decreased H3K27me3 or increased H3K4me1/H3K27ac in the promoters, or increased H3K27ac in the nearest enhancers. Together, 396 (41.7%) of the AH up-regulated genes were associated with chromatin changes in the promoters or enhancers. Distal only: the genes only associated with H3K27ac changes in the enhancers, but no changes of the 4 marks in the TSS±2kb regions; distal: the genes associated with H3K27ac changes in the enhancers as well as the changes of H3K27me3, H3K4me1 or H3K4me3 in the TSS±2kb regions.

FIG. 11. Active histone marks are enriched on CXCL 1, 6, and 8. The CXCL gene loci were examined in histone mark ChIP-seq. Representative AH and control (CON) samples are shown to demonstrate that active histone marks H3K27ac and H3K4me3 were increased at these loci whereas occupancy of repressive mark H3K27me3 decreased. This correlated with increased expression seen at mRNA level.

FIG. 12. FANTOM5 database CXCL chemokine expression levels in normal human livers. FANTOM5 human hg19 promoterome was accessed for genes CXCL1, 2, 6, and 8. Phase 1 and 2 pooled human tracks were accessed, filtered for liver related sources, and gene expression values from various tissue/cell sources were analyzed with mRNA CAGE sequencing. Values are plotted in relative log expression (RLE), and one-way ANOVA analysis was performed on the expression levels, with Post-hoc Dunnett's multiple comparison correction (n=3 for LSEC, HSC, and Hepatocyte sources and n=1 for fetal and adult liver tissues). Error bars indicate SD.

FIG. 13. Schematic of predicated NF-κB binding sites on CXCL promoters and super enhancer. Schematic of the CXCL locus was used to demonstrate the presence of NF-κB binding motifs in the CXCL promoter regions (labeled as P with gene name) or in CXCL SE (labeled as E sequentially based on distance away from CXCL8). TNFα treated LSEC H3K27ac ChIP-seq track was shown to highlight positions of activated chromosomal regions. The NF-κB binding site targeted for dCas9-KRAB suppression used in subsequent analysis was labeled as E1. Scale bar represents 50 kb.

FIGS. 14A-14C. Microfluidic chamber analysis of neutrophil adhesion demonstrates increased neutrophil in presence of LSEC secreted factors. FIG. 14A: Schematic of microfluidic chamber device. Cells are added to reservoir wells and flown through the chamber channels and drained from a connecting tubing. FIG. 14B: Neutrophils attached to LSECs lined chamber were quantified under various conditions. Neutrophils were labeled with Hoechst dye (round) and LSECs could also be seen (elongated cells). FIG. 14C: Quantification of neutrophil attachment normalized to control condition (n=4). Addition of CXCL1 (n=6) and LSEC medium (n=3) increased adhesion of neutrophils to LSECs. Arrows point to neutrophils. One-way ANOVA analysis was performed on the fold change ratio in cell counts, with Post-hoc Dunnett's multiple comparison correction. Error bars indicate SD.

FIGS. 15A-15B. Transwell neutrophil chemotaxis assay demonstrates increased chemotaxis with TNFα stimulated LSEC supernatant. FIG. 15A: IncuCyte obtained phase photos of LSECs (gray arrows) with attached neutrophils (white arrows) under various conditions. FIG. 15B: Quantification of neutrophil in lower chamber (chemotactic cells) per standard image field. Addition of CXCL1 recombinant protein or LSEC cultured medium increased neutrophil chemotaxis to lower chamber. Pretreatment of LSEC with TNFα further enhances neutrophil chemotaxis, while treatment with Celastrol diminished chemotaxis in a dose-dependent manner (n=3). One-way ANOVA analysis was performed on neutrophil cell counts, with Post-hoc Tukey's multiple comparison correction. Error bars indicate SD.

FIGS. 16A-16B. HEK293T lacks chromatin interaction with putative enhancer after TNFα stimulation. FIG. 16A: 4C was performed on TNFα stimulated LSEC cells and HEK293T cells. Interactions with CXCL1 promoter were plotted against fragment read count. A genome region of about 75 kb contained two peaks of CXCL1 interaction under TNFα stimulation (enclosed in the box) in LSECs. No interaction was seen in HEK293T cells. Viewpoint (VP) was labeled with blackline indicating the location of the reference sequence. Scale bar represents 50 kb. FIG. 16B: In silico analysis of HUVEC ChIP-seq for H3K27ac, NF-κB and BRD4 were obtained from public database, analyzed for this locus, and plotted under control conditions or TNFα stimulation. ⋅ indicates location of NF-κB site (E1) targeted for subsequent dCas9-KRAB experiments.

FIGS. 17A-17C. Identification of CXCL super enhancer in LSEC with H3K27ac ChIP-seq. FIGS. 17A: IGV snapshot of H3K27ac and NF-κB ChIP-seq signals in human LSECs and NF-κB ChIP-seq signals from HUVECs showing the CXCL super-enhancer site. H3K27ac occupancy in LSECs was enriched in the putative super-enhancer region and further increased after TNFα treatment. ChIP-seq of LSECs and HUVECs demonstrated enriched NF-κB binding in putative super-enhancer after TNFα treatment. There are lower signals of NF-κB ChIP-seq from LSECs compared to HUVECs due to technical limitations, but the most significant enrichment peaks were preserved and similar between the two groups. ⋅ indicates location of NF-κB site (E1) targeted for subsequent dCas9-KRAB experiments. Scale bar represents 50 kb. FIGS. 17B and 17C: ROSE algorithm of putative super-enhancer analysis from HUVEC cells without (FIG. 17B) or with (FIG. 17C) TNFα treatment. The top peaks in orange dashed box showed the most H3K27ac enrichment and are considered to be putative super-enhancers.

FIGS. 18A and 18B. NF-κB Binding and H3K27ac occupancy increase with TNFα stimulation. FIGS. 18A: ChIP-qPCR assays for NF-κB binding at various predicated NF-κB binding sites on CXCL promoters and SE were performed (as marked in FIG. 13). Sequence enrichment was normalized to percentage of input. There is enhancement of NF-κB binding at most of these sites with TNFα treatment CXCL1, 2, 3, 5, 8, E1, and E4). N=6 for CXCL1 and E1, n=4 for other samples. ChIP-qPCR isotype control with IgG showed minimal binding. Pair 1-test were performed for analysis. FIGS. 18B: ChIP-qPCR assays for H3K27ac occupancy at CXCL1 Promoter and CXCL SE E1 site (n=3). There is enrichment of H3K27ac at both loci, which is further increased after TNFα treatment and attenuated after celastrol treatment. Two-way matched-pairs ANOVA was performed with Post-hoc Tukey's multiple comparison correction. Error bar indicates SD.

FIG. 19. 3C chromatin conformation capture shows increased interaction with TNFα treatment that is unchanged with CRISPR targeting. 3C experiments were performed on control (black lines) or dCas9-KRAB cells targeting CXCL1 promoter (red lines) or CXCL SE (yellow lines) under control (solid lines) and TNFα treatment (dash lines) to detect binding of predicted CXCL super-enhancer with promoters of various CXCLs. E1 site (⋅) within the CXCL super-enhancer (vertical dash lines) was used as reference sequence. Interaction frequencies were plotted after normalized to that of RASSF6, a nearby noninflammatory gene used as control. Multiple other gene sequences between target CXCL promoters were selected as additional controls. X-axis maps relevant gene sequences as kb distance from RASSF6. Three-way ANOVA analysis was performed on the relative interaction frequencies separately with Control vs. dCas9-KRAB sgCXCL1 Promoter and Control vs. dCas9-KRAB sgCXCL SE, followed by Post-hoc Dunnett's multiple comparison correction (n=3). There was no significant change to SE-promoter interactions with either TNFα treatment or dCas9-KRAB targeting. There are increased gene interactions at CXCL8 and CXCL1 compared to negative control.

FIG. 20. Selection of sgRNA for CRISPR dCas9-KRAB targeting. dCas9-KRAB fusion protein targeting CXCL super-enhancer NFκB site suppressed CXCL expression in LSECs. 15 sgRNAs targeting one of four top predicted NFκB binding sites on the CXCL super-enhancer were studied here in dCas9-KRAB treated cells. For simplicity of presentation, the sgRNAs with best activity targeting each NFκB binding site was shown alongside with control sgRNA (green box), CXCL1 promoter sgRNA (red box), n=4. SE sgRNA decreased CXCL1, 3, 6, and 8 expression to varying degrees (qPCR), but did not affect expression of MTHFD2L, a nearby noninflammatory gene (negative control). Of these sgRNAs targeting CXCL SE, sgRNA targeting E1 showed best efficacy and consistency of CXCL deduction and was used for subsequent analysis (yellow box). Changes in chemokine expression were calculated as fold change over basal expression and log₁₀ (fold change) was plotted on the y-axis. Two-way ANOVA analysis was performed on the log-transformed ratios followed by Post-hoc Sidak's multiple comparison correction. Error bars indicate SD.

FIG. 21. CRISPR dCas9-KRAB LSEC cytotoxicity assay. IncuCyte system was utilized to image cells and assess for cell toxicity. Dying cells (white arrows) are stained green by fluorescent dye, and green cells are counted for each low power image field. Images shown were acquired after 4 hours of incubation with dye. Quantification for the number of dead cells was done at 6 hour intervals for 24 hours, showing no difference among control, P1 sgRNA/dCas9-KRAB cotransfected cells, and E1 sgRNA/dCas9-KRAB cotransfected cells. Error bars indicate SD, n=3. Two-way RM ANOVA analysis showed no significant difference among treatment groups.

FIGS. 22A-22D. dCas9-KRAB fusion protein targeting CXCL super enhancer NFκB site suppressed CXCL expression in LSECs. FIG. 22A: dCas9-KRAB treatment with sgRNA targeting CXCL super-enhancer (E1) decreased CXCL1, 6, and 8 expression, but did not affect expression of CXCL2, 3, 5, and MTHFD2L, a nearby noninflammatory gene (negative control). Log₁₀ (fold change) was plotted, n=4. FIG. 22B: dCas9-KRAB treatment with sgRNA targeting CXCL1 promoter (P1) decreased expression of CXCL1, but not other CXCLs by qPCR, n=4. FIGS. 22C and 22D: sgRNA treatment alone targeting either CXCL1 promoter or CXCL SE without dCas9-KRAB did not result in significant repression of CXCL expression. Two-way matched-pairs ANOVA was performed on log-transformed fold-change values with Post-hoc Tukey's multiple comparison correction. Error bars indicate SD, n=4.

FIGS. 23A and 23B. dCas9-FLAG transduced cells suppressed CXCL expression. FIG. 23A: Cotransfection of dCas9-FLAG with E1 sgRNA decreased expression of CXCL1 (n=7), and but not CXCL6 (n=4), CXCL8 (n=6), or MTHFD2L (n=6) by qPCR. Changes in chemokine expression were calculated as fold change over basal expression, and log₁₀ (fold change) was plotted on the y-axis. FIG. 23B: Cotransfection of dCas9-FLAG with P1 sgRNA decreased CXCL1 (n=7) expression at basal level and after TNFα treatment. There was no effect on expression of CXCL6 (n=4), CXCL8 (n=6), or MTHFDL2 (n=4). Two-way matched-pairs ANOVA was performed on log-transformed fold-change values, with Post-hoc Tukey's multiple comparison correction. Error bars indicate SD.

FIG. 24. Bromodomain inhibitors suppress expression of CXCLs. LSECs were pretreated with UMN627 (0-50 μM; n=4). CXCL2, 3, and 5 expression levels were assessed by qPCR. Expression levels were normalized to basal condition and log₁₀ fold change values were plotted. One-way matched-pairs ANOVA analysis was performed with Post-hoc Dunnett's multiple comparison correction. Error bars indicate SD. There were linear trends for decreasing CXCL2, 3, 5 expression with increasing UMN627 concentrations with TNFα, (p<0.01 for all groups).

FIGS. 25A-25C. Multiple human cell types demonstrate looping interactions between CXCL super enhancer and CXCL promoters. FIG. 25A: Comparison of HUVEC and LSEC in the levels of cis-interactions involving CXCL1 promoter. Heatmap represented the differences in chromosomal interaction frequency (red, higher signal in LSECs; blue, higher signal in HUVEC), showing a similar interaction profile between the two cell types. Arc graphs represented chromosomal interactions of CXCL1 promoter, most notably with CXCL super-enhancer (gold bar) in the two cell types. Data were accessed from the 3DIV Hi-C database. FIG. 25B: Comparison of CXCL1 promoter chromosomal interactions under control condition and TNFα stimulation in IMR90 fibroblast as determined by Hi-C. Heatmap represented the differences in chromosomal interaction with or without TNFα treatment (red, higher signal in TNFα treated cells), which showed increased cis-interactions following TNFα treatment. Arc graphs depicted chromosomal interactions of the CXCL1promoter, which were enhanced after TNFα stimulation, particularly with the CXCL super-enhancer (gold bar). Data were accessed from 3DIV. FIG. 25C: Comparison of chromosomal interactions involving CXCL1 promoter across three primary human cell types. Elevated chromosomal interactions were identified with the CXCL super-enhancer. Capture Hi-C data were from the CHiCP web browser (chicp.org). In the three plots, CXCL super-enhancer was marked in gold color.

FIG. 26. The CXCL locus contained putative super enhancers in mouse macrophage and hepatocyte. IGV snapshot of NF-κB, BRD4, and H3K27ac ChIP-seq signals in mouse macrophage and hepatocyte. The two putative super-enhancers (enclosed by red box) were identified. LPS treatment of macrophage cells increased the binding of NF-κB and BRD4 at this locus. ChIP-seq of mouse hepatocytes demonstrated enriched NF-κB binding in putative super-enhancer after IL1β treatment. H3K27ac occupancy in hepatocytes was enriched in the putative super-enhancer region and further increased after IL1β treatment (pink, baseline condition; teal, IL1β treatment). Scale bar represents 20 kb.

FIG. 27A and 27B. TNFα and LPS treatments of human peripheral blood monocytes derived macrophages significantly increased expression of CXCL chemokines by qPCR. Monocytes were cultured with M-CSF to induce differentiation into macrophages. Cells at Day 3 (FIG. 27A) and 7 (FIG. 27B) of culture were assayed separately. CXCL chemokines expression are shown as fold change over control condition for qPCR in log scale. One-way ANOVA analysis of log transformed ratios was performed with Post-hoc Dunnett's multiple comparison correction. Error bars indicate SD.

FIGS. 28A-28D. Alcohol feeding increased neutrophil infiltration in NIAAA chronic binge alcohol feeding model. FIG. 28A: qPCRs of CXCL chemokine expression and neutrophil marker Ly6g with alcohol feeding. Expression levels were normalized to average expression of pair-fed control mice. FIG. 28B: Serum ALT levels were shown. No significance was seen between groups for FIG. 28A or FIG. 28B. FIG. 28C: IHC for MPO (neutrophil marker) showed a significantly increased amount of neutrophil infiltration with alcohol feeding. FIG. 28D: Frozen sections of mouse liver were stained with Oil-Red-O (red). Hematoxylin counterstain was used to stain nuclei (blue). Quantification showed an increase in steatosis with alcohol. For all analysis one-way ANOVA was performed on log-transformed normalized expression values or cell counts for IHC staining or ALT values, with Post hoc Tukey's multiple comparison correction used for above analysis. N=6 for each group, except for alcohol-fed mice ALT measurement n=5 (serum was unable to be collected from one mouse for technical reasons).

FIGS. 29A and 29B. Comparison of LPS alone treatment with combination alcohol-feeding/LPS treatment mice. FIG. 29A: qPCRs of CXCL chemokine and neutrophil marker Ly6g were normalized to average expression of Pair-fed control mice (n=12). Increased Cxcl1 and Cxcl2 expression was seen in combination group (n=12) compared to LPS alone (n=10). No significant difference was seen with Ly6g. FIG. 29B: BODIPY 493/503 staining of mice liver was performed. Scatter plot shows normalized quantification of BODIPY 493/503 staining to Pair-fed mice (n=12 for Pair-fed, n=14 for EtOH+LPS, and n=10 for LPS only). One-way ANOVA was performed with qPCR fold changes or BODIPY quantification ratios, with Post-hoc Sidak's multiple comparison correction. Error bars indicate SD.

FIG. 30. BODIPY 493/503 stain for analysis of steatosis in alcohol gavage/LPS injection mice treated with UMN627. Frozen section of mouse liver was stained with BODIPY 493/503 (shown in green). DAPI was used to stain nuclei (shown in blue). Scattered plot showed normalized quantification of BODIPY 493/503 staining (n=7 Control/Veh, n=10 Binge+LPS/veh, n=7 Control/UMN627, n=12 Binge+LPS/UMN627). Two-way ANOVA with Tukey's correction was performed on normalized quantification ratios, and BODIPY 493/503 stain was not significantly correlated with Binge+LPS treatment or UMN627 injections. Error bars indicate SD.

FIG. 31. FANTOM5 Database CXCL Chemokine Expression Levels in Normal Mouse Livers. FANTOM5 mouse mm9 promoterome was access for genes Cxcl1 and 2. Phase 1 and 2 pooled mouse tracks were accessed, filtered for liver related sources, and gene expression values from various tissue/cell sources were analyzed. Values are plotted in relative log expression (RLE), and one-way ANOVA analysis was performed on the expression levels, with Post-hoc Dunnett's multiple comparison correction (n=4 for LSEC and HSC, n=9 for Hepatocyte). Error bars indicate SD.

FIGS. 32A and 32B. Quantitative PCR of chemokine expression in LPS treated HHSEC with BRD4 inhibitors. FIG. 32A: qPCR expression of CXCL1, normalized to control, with three concentrations: 0.31 μM, 1.25 μM, and 5 μM. FIG. 32B: qPCR expression of CCL2, normalized to control, with three concentrations: 0.31 μM, 1.25 μM, and 5 μM. Each concentration was performed in triplicate (n=3).

FIGS. 33A and 33B. HHSEC viability at various concentrations of BRD4inhibitors. FIG. 33A: Dose response curves of HHSEC viability (i.e., absorbance with respect to control) at several concentrations of BRD4 inhibitors. Two graphs were generated to make compounds more visible. Every data point is the mean±SEM (n=3). FIG. 33B: Concentration of BRD4 inhibitors that cause 50% toxicity to HHSEC (i.e., EC₅₀). Data are represented as mean±SEM (n=3). Significance was determined with a one-way ANOVA combined with a Tukey post hoc test. P-values<0.0001.

FIGS. 34A and 34B. UMN627 inhibition of inflammatory chemokine expression in murine LPS infection models. FIG. 34A: qPCR analysis showing CXCL1 mRNA expression of control, LPS only, and LPS+UMN627 groups. UMN627 was administered one hour before LPS at several concentrations (mg drug/kg mouse). IC₅₀=10.9 mg/kg. FIG. 34B: CCL2 mRNA expression of control, LPS only, and LPS+UMN627 groups. IC₅₀=8.8 mg/kg. No significant differences were found. Significance was determined with a one-way ANOVA followed by a Dunnett post hoc test between LPS control and UMN627 treatments. p-values: *=p<0.05

FIGS. 35A and 35B. AC4118 Inhibition of Inflammatory Chemokine Expression in Murine LPS Infection Models. FIG. 35A: qPCR analysis showing CXCL1 mRNA expression of control, LPS only, and LPS+AC4118 groups. AC4118 was administered one hour before LPS at several concentrations (mg drug/kg mouse). FIG. 35B: CCL2 mRNA expression of control, LPS only, and LPS+AC4118 groups. Significance was determined with a one-way ANOVA followed by a Dunnett post hoc test between LPS control and Ac4118 treatments. p-values: *=p<0.05, ***=p<0.001.

### DETAILED DESCRIPTION

This document provides methods and materials for treating mammals (e.g., humans) having a liver disease (e.g., an ALD such as AH). For example, this document provides inhibitors of a BRD4 polypeptide as well as methods for using inhibitors of a BRD4 polypeptide. In some cases, one or more (e.g., one, two, three, four, or more) inhibitors of a BRD4 polypeptide can be administered to a mammal (e.g., a human) having a liver disease (e.g., an ALD such as AH) to treat the mammal.

In some cases, one or more (e.g., one, two, three, four, or more) inhibitors of a BRD4 polypeptide can be used to reduce the severity of one or more symptoms of the liver disease. For example, one or more inhibitors of a BRD4 polypeptide can be administered to a mammal (e.g., a human) in need thereof (e.g., a human having a liver disease (e.g., an ALD such as AH)) to reduce the severity of one or more symptoms of the liver disease. Examples of symptoms of an ALD include, without limitation, anorexia, weight loss, abdominal pain, abdominal distention, nausea, vomiting, hepatomegaly, jaundice, angiomas (e.g., spider angiomas), fever, encephalopathy, thrombocytopenia, hypoalbuminemia, coagulopathy, fatigue, weakness, liver failure, bleeding complications, lower extremity swelling, and kidney dysfunction. In some cases, the materials and methods described herein can be effective to reduce the severity of one or more symptoms of ALD in a mammal having an ALD by, for example, 10, 20, 30, 40, 50, 60, 70, 80, 90, 95, or more percent.

In some cases, one or more (e.g., one, two, three, four, or more) inhibitors of a BRD4 polypeptide can be used to reduce the severity of one or more complications associated with the ALD. For example, one or more inhibitors of a BRD4 polypeptide can be administered to a mammal (e.g., a human) in need thereof (e.g., a human having a liver disease (e.g., an ALD such as AH)) to reduce the severity of one or more complications associated with the ALD. Complications associated with an ALD can include, without limitation, enlarged veins (varices), ascites, hepatic encephalopathy, kidney failure, infection, fatigue, weakness, liver failure, bleeding complications, lower extremity swelling, and kidney dysfunction. In some cases, the materials and methods described herein can be effective to reduce the severity of one or more complications associated with an ALD in a mammal having an ALD by, for example, 10, 20, 30, 40, 50, 60, 70, 80, 90, 95, or more percent.

In some cases, one or more (e.g., one, two, three, four, or more) inhibitors of a BRD4 polypeptide can be used to increase the survival of the mammal. For example, one or more inhibitors of a BRD4 polypeptide can be administered to a mammal (e.g., a human) in need thereof (e.g., a human having a liver disease (e.g., an ALD such as AH)) to increase the survival of the mammal. In some cases, the materials and methods described herein can be effective to increase the survival of a mammal having an ALD (e.g., having an ALD and identified as being at high risk of mortality) by, for example, 10, 20, 30, 40, 50, 60, 70, 80, 90, 95, or more percent.

In some cases, one or more (e.g., one, two, three, four, or more) inhibitors of a BRD4 polypeptide can be used to reduce or eliminate inflammation in the liver of the mammal. For example, one or more inhibitors of a BRD4 polypeptide can be administered to a mammal (e.g., a human) in need thereof (e.g., a human having a liver disease (e.g., an ALD such as AH)) to reduce or eliminate inflammation in the liver of the mammal. In some cases, the materials and methods described herein can be effective to reduce inflammation in the liver of a mammal having an ALD by, for example, 10, 20, 30, 40, 50, 60, 70, 80, 90, 95, or more percent.

In some cases, one or more (e.g., one, two, three, four, or more) inhibitors of a BRD4 polypeptide can be used to reduce the number of neutrophils in the liver of the mammal. For example, one or more inhibitors of a BRD4 polypeptide can be administered to a mammal (e.g., a human) in need thereof (e.g., a human having a liver disease (e.g., an ALD such as AH)) to reduce the number of neutrophils in the liver of the mammal. In some cases, the materials and methods described herein can be effective to reduce the number of neutrophils in the liver of a mammal having an ALD by, for example, 10, 20, 30, 40, 50, 60, 70, 80, 90, 95, or more percent.

Any appropriate mammal having a liver disease (e.g., an ALD such as AH) can be treated as described herein (e.g., by administering one or more inhibitors of a BRD4 polypeptide). Examples of mammals that can have a liver disease (e.g., an ALD such as AH) and can be treated as described herein include, without limitation, humans, non-human primates such as monkeys, dogs, cats, horses, cows, pigs, sheep, mice, and rats. In some cases, a human can be treated as described herein.

When treating a mammal (e.g., a human) having a liver disease, the mammal can have any type of liver disease. Examples of liver diseases that can be treated as described herein (e.g., by administering one or more inhibitors of a BRD4 polypeptide) include, without limitation, ALDs (e.g., AH), autoimmune liver diseases, cholestatic liver diseases, nonalcoholic fatty liver diseases (NAFLDs), nonalcoholic steatohepatitis (NASH), and inflammatory liver diseases. In some cases, a liver disease that can be treated as described herein (e.g., by administering one or more inhibitors of a BRD4 polypeptide) can be as described elsewhere (see, e.g., Gilan et al., Science, 368 (6489): 387-394 (2020)).

In some cases, the methods described herein can include identifying a mammal (e.g., a human) as having a liver disease (e.g., an ALD such as AH). Any appropriate method can be used to identify a mammal as having a liver disease (e.g., an ALD such as AH). For example, circulating extracellular vesicles and their sphingolipid content, elevated AST, elevated ALT, elevated bilirubin, INR with a clinical history of alcohol intake, jaundice, abdominal distension, lower extremity swelling, spider angiomas in patients with a history of alcohol use, and/or liver biopsy can be used to identify mammals (e.g., humans) having a liver disease (e.g., an ALD such as AH).

A mammal (e.g., a human) having a liver disease (e.g., an ALD such as AH) can be administered or instructed to self-administer any one or more (e.g., one, two, three, four, or more) inhibitors of a BRD4 polypeptide. An inhibitor of a BRD4 polypeptide can be an inhibitor of BRD4 polypeptide activity or an inhibitor of BRD4 polypeptide expression. In some cases, an inhibitor of a BRD4 polypeptide can be an inhibitor of the bromodomain 1 (BD1) of the BRD4 polypeptide. In some cases, an inhibitor of a BRD4 polypeptide can be an inhibitor of the BD2 of the BRD4 polypeptide. In some cases, an inhibitor of a BRD4 polypeptide can be an inhibitor of both the BD1 and the BD2 of the BRD4 polypeptide.

Examples of compounds that can reduce or eliminate BRD4 polypeptide activity include, without limitation, anti-BRD4 antibodies (e.g., neutralizing anti-BRD4 antibodies), small molecules that target (e.g., target and bind) to a BRD4 polypeptide, and chemicals that can lead to the degradation of a BRD4 polypeptide. When a compound that can reduce or eliminate BRD4 polypeptide activity is a small molecule that targets (e.g., targets and binds) to a BRD4 polypeptide, the small molecule can be in the form of salt (e.g., a pharmaceutically acceptable salt). Examples of compounds that can reduce or eliminate BRD4 polypeptide expression include, without limitation, nucleic acid molecules designed to induce RNA interference of BRD4 polypeptide expression (e.g., a siRNA molecule or a shRNA molecule), antisense molecules, and miRNAs. In some cases, an inhibitor of a BRD4 polypeptide can be as described elsewhere (see, e.g., Ding et al., Proc. Natl. Acad. Sci., 112 (51): 15713-15718 (2015); Khan et al., PLOS One, April 23; 9 (4): e95051 (2014); Felgenhauer et al., Neoplasia, Oct;20 (10): 965-974 (2018); Waring et al., Nat. Chem. Biol., 12:1097-1104 (2016); and Gilan et al., Science, 368 (6489): 387-394 (2020)). In some cases, an inhibitor of BRD4 polypeptide activity that can be used to treat a liver disease as described herein can have the structure of Formula (I):

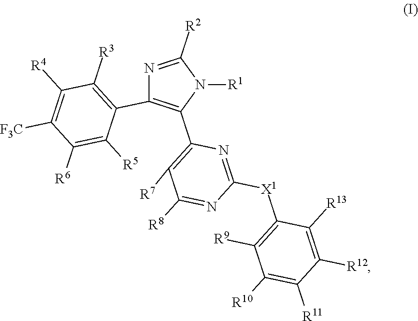

or a pharmaceutically acceptable salt thereof, wherein:
R¹ is a 4-7-membered heterocycloalkyl ring comprising at least one N atom, which is optionally substituted with 1, 2, or 3 R^{A} substituents independently selected from C₁₋₆ alkyl, C₂₋₆ alkenyl, C₂₋₆ alkynyl, C₁₋₄ haloalkyl, and C₃₋₅ cycloalkyl, each of which is optionally substituted with 1, 2, or 3 substituents independently selected from OH, NO₂, CN, amino, C₁₋₆ alkylamino, and di(C₁₋₆ alkyl)amino;
X¹ is selected from O and NR^{N},
R^{N} is selected from H, C₁₋₃ alkyl, and C₁₋₃ haloalkyl; and each of R², R³, R⁴, R⁵, R⁶, R⁷, R⁸, R⁹, R¹⁰, R¹¹, R¹², and R¹³ is independently selected from H, OH, NO₂, CN, halo, C₁₋₆ alkyl, C₂₋₆ alkenyl, C₂₋₆ alkynyl, C₁₋₄ haloalkyl, C₁₋₆ alkoxy, and C₁₋₆ haloalkoxy, wherein each of said C₁₋₆ alkyl, C₂₋₆ alkenyl, and C₂₋₆ alkynyl is substituted with a substituent independently selected from OH, NO₂, CN, amino, C₁₋₆ alkylamino, and di(C₁₋₆ alkyl)amino.

In some embodiments, X¹ is O.

In some embodiments, X¹ is NR^{N}.

In some embodiments, R^{N} is selected from H and C₁₋₃ alkyl.

In some embodiments, the compound of Formuyla (I) has formula:

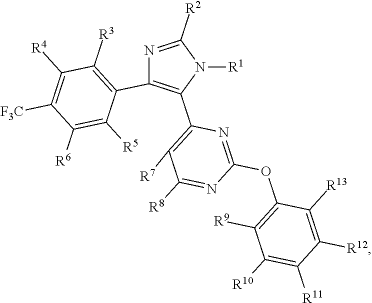

or a pharmaceutically acceptable salt thereof.

In some embodiments, the compound of Formula (I) has formula:

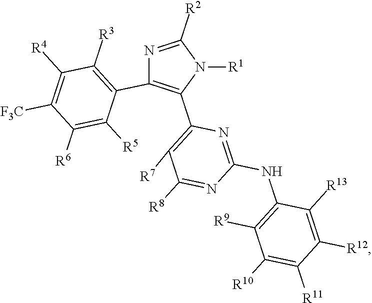

or a pharmaceutically acceptable salt thereof.

In some embodiments, R¹ is a 4-7-membered heterocycloalkyl ring comprising at least one N atom, which is optionally substituted with an R^{A} substituent.

In some embodiments, R¹ is selected from azetidine, pyrrolidine, piperidine, morpholine, thiomorpholine, imidazolidine, pyrazolidine, and oxazolidine, each of which is optionally substituted with an R^{A}.

In some embodiments, R¹ is selected from pyrrolidine and piperidine, each of which is optionally substituted with an R^{A}.

In some embodiments, R^{A} is selected from C₁₋₆ alkyl, C₂₋₆ alkenyl, and C₂₋₆ alkynyl, each of which is optionally substituted with a substituent selected from amino, C₁₋₆ alkylamino, and di(C₁₋₆ alkyl)amino.

In some embodiments, R^{A} is C₁₋₆ alkyl, substituted with a substituent selected from amino, C₁₋₆ alkylamino, and di(C₁₋₆ alkyl)amino.

In some embodiments, each of R², R³, R⁴, R⁵, R⁶, R⁷, R⁸, R⁹, R¹⁰, R¹¹, R¹², and R¹³ is independently selected from H, OH, NO₂, CN, halo, C₁₋₆ alkyl, C₂₋₆ alkenyl, and C₂₋₆ alkynyl.

In some embodiments, each of R², R³, R⁴, R⁵, R⁶, R⁷, R⁸, R⁹, R¹⁰, R¹¹, R¹², and R¹³ is independently selected from H, halo, and C₁₋₆ alkyl.

In some embodiments, the compound of Formula (I) has formula:

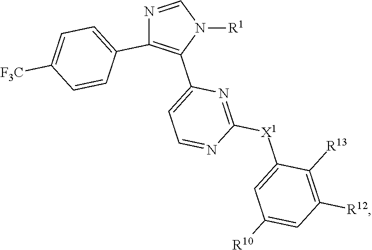

or a pharmaceutically acceptable salt thereof.

In some embodiments, the compound of Formula (I) has formula:

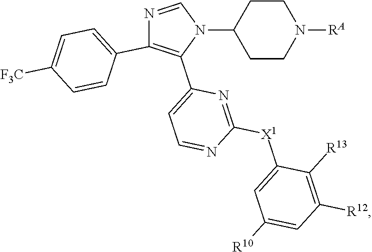

or a pharmaceutically acceptable salt thereof.

In some embodiments, the compound of Formula (I) has formula:

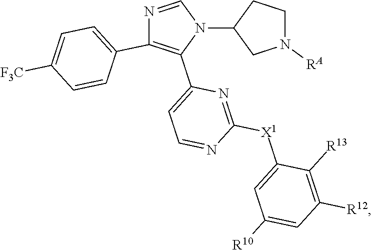

or a pharmaceutically acceptable salt thereof.

In some embodiments, the compound of Formula (I) is selected from any one of the following compounds:

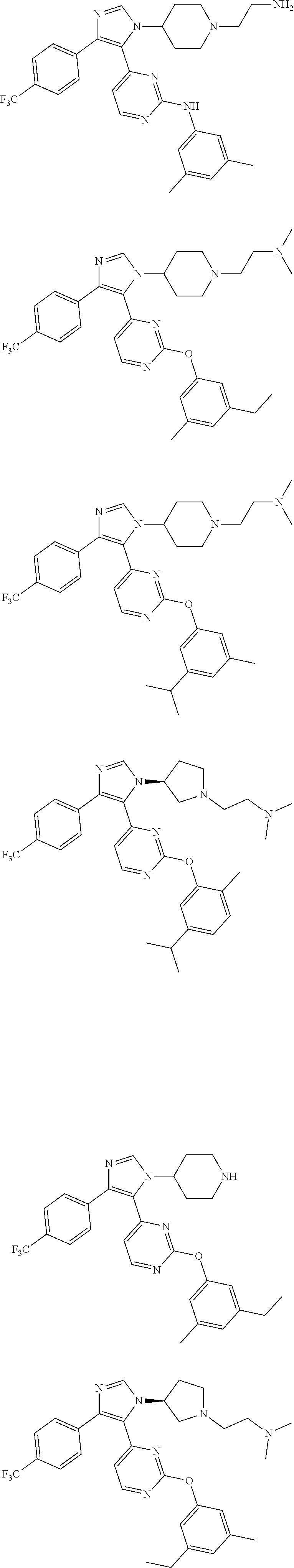 [SMILES: Cc1cc(C)cc(Nc2nccc(-c3c(-c4ccc(C(F)(F)F)cc4)ncn3C3CCN(CCN)CC3)n2)c1 ; CCc1cc(C)cc(Oc2nccc(-c3c(-c4ccc(C(F)(F)F)cc4)ncn3C3CCN(CCN(C)C)CC3)n2)c1 ; Cc1cc(Oc2nccc(-c3c(-c4ccc(C(F)(F)F)cc4)ncn3C3CCN(CCN(C)C)CC3)n2)cc(C(C)C)c1 ; Cc1ccc(C(C)C)cc1Oc1nccc(-c2c(-c3ccc(C(F)(F)F)cc3)ncn2[C@H]2CCN(CCN(C)C)C2)n1 ; CCc1cc(C)cc(Oc2nccc(-c3c(-c4ccc(C(F)(F)F)cc4)ncn3C3CCNCC3)n2)c1 ; CCc1cc(C)cc(Oc2nccc(-c3c(-c4ccc(C(F)(F)F)cc4)ncn3[C@H]3CCN(CCN(C)C)C3)n2)c1]

or a pharmaceutically acceptable salt thereof.

In some cases, an inhibitor of BRD4 polypeptide activity that can be used to treat a liver disease as described herein can have the structure of Formula (II):

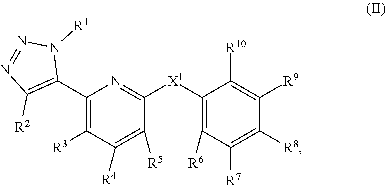

or a pharmaceutically acceptable salt thereof, wherein:
R¹ is a 4-7-membered heterocycloalkyl ring comprising at least one N atom, which is optionally substituted with 1, 2, or 3 R^{A} substituents independently selected from C₁₋₆ alkyl, C₂₋₄ alkenyl, C₂₋₄ alkynyl, C₁₋₄ haloalkyl, and C₃₋₅ cycloalkyl, each of which is optionally substituted with 1, 2, or 3 substituents independently selected from OH, NO₂, CN, amino, C₁₋₆ alkylamino, and di(C₁₋₆ alkyl)amino;
X¹ is selected from O and NR^{N};
R^{N} is selected from H, C₁₋₃ alkyl, and C₁₋₃ haloalkyl; and
each of R², R³, R⁴, R⁵, R⁶, R⁷, R⁸, R⁹, and R¹⁰ is independently selected from H, OH, NO₂, CN, halo, C₁₋₆ alkyl, C₂₋₆ alkenyl, C₂₋₆ alkynyl, C₁₋₄ haloalkyl, C₁₋₆ alkoxy, and C₁₋₆ haloalkoxy, wherein each of said C₁₋₆ alkyl, C₂₋₆ alkenyl, and C₂₋₆ alkynyl is substituted with a substituent independently selected from OH, NO₂, CN, amino, C₁₋₆ alkylamino, and di(C₁₋₆ alkyl)amino.

In some embodiments, X¹ is O.

In some embodiments, X¹ is NR^{N}.

In some embodiments, R^{N} is selected from H and C₁₋₃ alkyl.

In some embodiments, the compound of Formula (II) has formula:

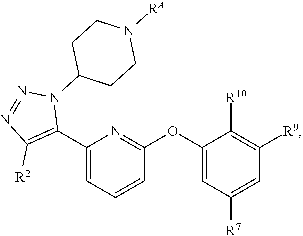

or a pharmaceutically acceptable salt thereof.

In some embodiments, R¹ is a 4-7-membered heterocycloalkyl ring comprising at least one N atom, which is optionally substituted with an RA substituent.

In some embodiments, R¹ is selected from azetidine, pyrrolidine, piperidine, morpholine, thiomorpholine, imidazolidine, pyrazolidine, and oxazolidine, each of which is optionally substituted with an R^{A}.

In some embodiments, R¹ is selected from pyrrolidine and piperidine, each of which is optionally substituted with an R^{A}.

In some embodiments, R¹ is piperidine, optionally substituted with an R^{A}.

In some embodiments, R^{A} is selected from C₁₋₆ alkyl, C₂₋₆ alkenyl, and C₂₋₆ alkynyl, each of which is optionally substituted with a substituent selected from amino, C₁₋₆ alkylamino, and di(C₁₋₆ alkyl)amino.

In some embodiments, R^{A} is C₁₋₆ alkyl, substituted with a substituent selected from amino, C₁₋₆ alkylamino, and di(C₁₋₆ alkyl)amino.

In some embodiments, each of R², R³, R⁴, R⁵, R⁶, R⁷, R⁸, R⁹, and R¹⁰ is independently selected from H, OH, NO₂, CN, halo, C₁₋₆ alkyl, C₂₋₆ alkenyl, and C₂₋₆ alkynyl.

In some embodiments, each of R², R³, R⁴, R⁵, R⁶, R⁷, R⁸, R⁹, and R¹⁰ is independently selected from H, halo, and C₁₋₆ alkyl.

In some embodiments, the compound of Formula (II) has formula:

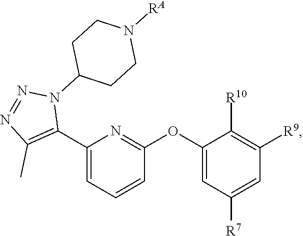

or a pharmaceutically acceptable salt thereof.

In some embodiments, the compound of Formula (II) is selected from any one of the following compounds:

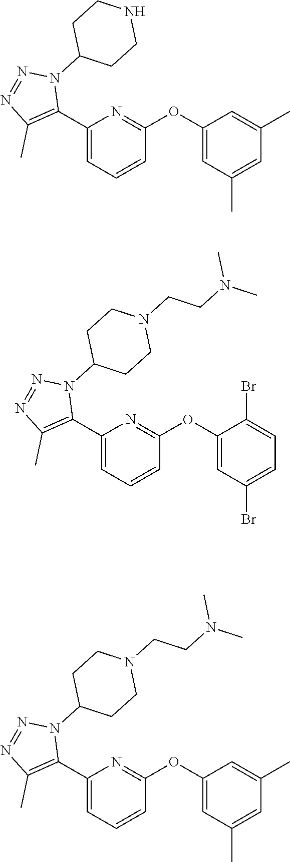

or a pharmaceutically acceptable salt thereof.

In some cases, an inhibitor of BRD4 polypeptide activity that can be used to treat a liver disease as described herein can have the structure of Formula (III):

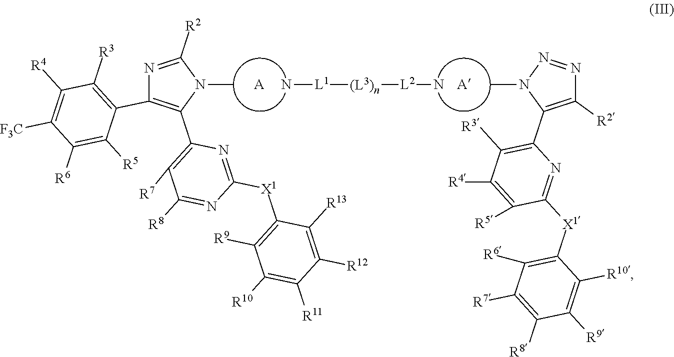

or a pharmaceutically acceptable salt thereof, wherein:
each of ring A and ring A′ is independently a 4-7-membered heterocycloalkyl ring comprising at least one N atom, which is optionally substituted with 1, 2, or 3 RA substituents independently selected from C₁₋₆ alkyl, C₂₋₆ alkenyl, C₂₋₆ alkynyl, C₁₋₄ haloalkyl, and C₃₋₅ cycloalkyl, each of which is optionally substituted with 1, 2, or 3 substituents independently selected from OH, NO₂, CN, amino, C₁₋₆ alkylamino, and di(C₁₋₆ alkyl)amino;
each of X¹ and X¹′ is independently selected from O and NR^{N};
each R^{N} is independently selected from H, C₁₋₃ alkyl, and C₁₋₃ haloalkyl;
each of R², R³, R⁴, R⁵, R⁶, R⁷, R⁸, R⁹, R¹⁰, R¹¹, R¹², R¹³, R^{2′}, R^{3′}, R^{4′}, R^{5′}, R^{6′}, R^{7′}, R^{8′}, R^{9′}, and R^{10′} is independently selected from H, OH, NO₂, CN, halo, C₁₋₆ alkyl, C₂₋₆ alkenyl, C₂₋₆ alkynyl, C₁₋₄ haloalkyl, C₁₋₆ alkoxy, and C₁₋₆ haloalkoxy, wherein each of said C₁₋₆ alkyl, C₂₋₆ alkenyl, and C₂₋₆alkynyl is substituted with a substituent independently selected from OH, NO₂, CN, amino, C₁₋₆ alkylamino, and di(C₁₋₆ alkyl)amino;
each of L¹ and L² is independently C₁₋₃ alkylene, optionally substituted with 1, 2, or 3 substituents independently selected from OH, NO₂, CN, halo, amino, and carboxy;
n is an integer selected from 1 to 10; and
each L³ is independently selected from C(═O), N(R^{N}), O, (—C₁₋₃ alkylene-O—)ₓ, (—O—C₁₋₃ alkylene-)ₓ, and —C₁₋₃ alkylene-, wherein each x is independently an integer from 1 to 10 and each C₁₋₃ alkylene is optionally substituted with 1, 2, or 3 substituents independently selected from OH, NO₂, CN, halo, amino, and carboxy.

In some embodiments, X¹ is O.

In some embodiments, X¹ is NR^{N}.

In some embodiments, X¹ is O.

In some embodiments, X¹ is NR^{N}.

In some embodiments, R^{N} is selected from H and C₁₋₃ alkyl.

In some embodiments, ring A is a 4-7-membered heterocycloalkyl ring comprising at least one N atom, which is optionally substituted with an RA substituent.

In some embodiments, ring A is selected from azetidine, pyrrolidine, piperidine, morpholine, thiomorpholine, imidazolidine, pyrazolidine, and oxazolidine, each of which is optionally substituted with an R^{A}.

In some embodiments, ring A is selected from pyrrolidine and piperidine, each of which is optionally substituted with an R^{A}.

In some embodiments, ring A′ is a 4-7-membered heterocycloalkyl ring comprising at least one N atom, which is optionally substituted with an R^{A} substituent.

In some embodiments, ring A′ is selected from azetidine, pyrrolidine, piperidine, morpholine, thiomorpholine, imidazolidine, pyrazolidine, and oxazolidine, each of which is optionally substituted with an R^{A}.

In some embodiments, ring A′ is selected from pyrrolidine and piperidine, each of which is optionally substituted with an R^{A}.

In some embodiments, R^{A} is selected from C₁₋₆ alkyl, C₂₋₆ alkenyl, and C₂₋₆ alkynyl, each of which is optionally substituted with a substituent selected from amino, C₁₋₆ alkylamino, and di(C₁₋₆ alkyl)amino.

In some embodiments, RA is C₁₋₆ alkyl, substituted with a substituent selected from amino, C₁₋₆ alkylamino, and di(C₁₋₆ alkyl)amino.

In some embodiments, each of R², R³, R⁴, R⁵, R⁶, R⁷, R⁸, R⁹, R¹⁰, R¹¹, R¹², R¹³, R^{2′}, R^{3′}, R^{4′}, R^{5′}, R^{6′}, R^{7′}, R^{8′}, R^{9′}, and R^{10′} is independently selected from H, OH, NO₂, CN, halo, C₁₋₆ alkyl, C₂₋₆ alkenyl, and C₂₋₆ alkynyl.

In some embodiments, each of R², R³, R⁴, R⁵, R⁶, R⁷, R⁸, R⁹, R¹⁰, R¹¹, R¹², R¹³, R^{2′}, R^{3′}, R^{4′}, R^{5′}, R^{6′}, R^{7′}, R^{8′}, R^{9′}, and R^{10′} is independently selected from H, halo, and C₁₋₆ alkyl.

In some embodiments, L¹ is C₁₋₃ alkylene.

In some embodiments, L² is C₁₋₃ alkylene.

In some embodiments, n is 2, 3, 4, 5, 6, 7, or 8.

In some embodiments, L³ is C(═O).

In some embodiments, L³ is N(R^{N}).

In some embodiments, L³ is O.

In some embodiments, L³ is (—C₁₋₃ alkylene-O—)ₓ.

In some embodiments, L³ is (—O—C₁₋₃ alkylene-)ₓ.

In some embodiments, L³ is —C₁₋₃ alkylene-.

In some embodiments, x is 2, 3, 4, 5, 6, 7, or 8.

In some embodiments, the compound of Formula (III) has formula:

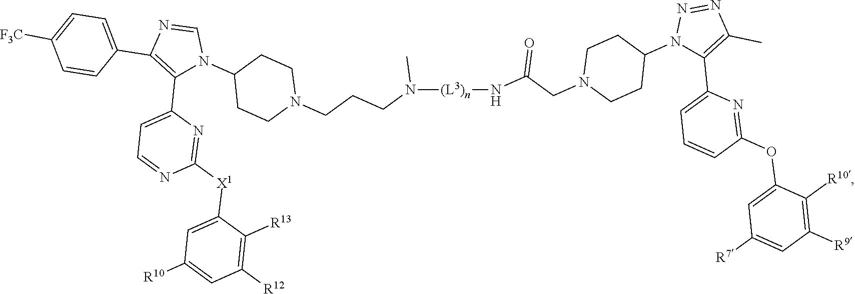

or a pharmaceutically acceptable salt thereof.

In some embodiments, the compound of Formula (III) is selected from any one of the following compounds:

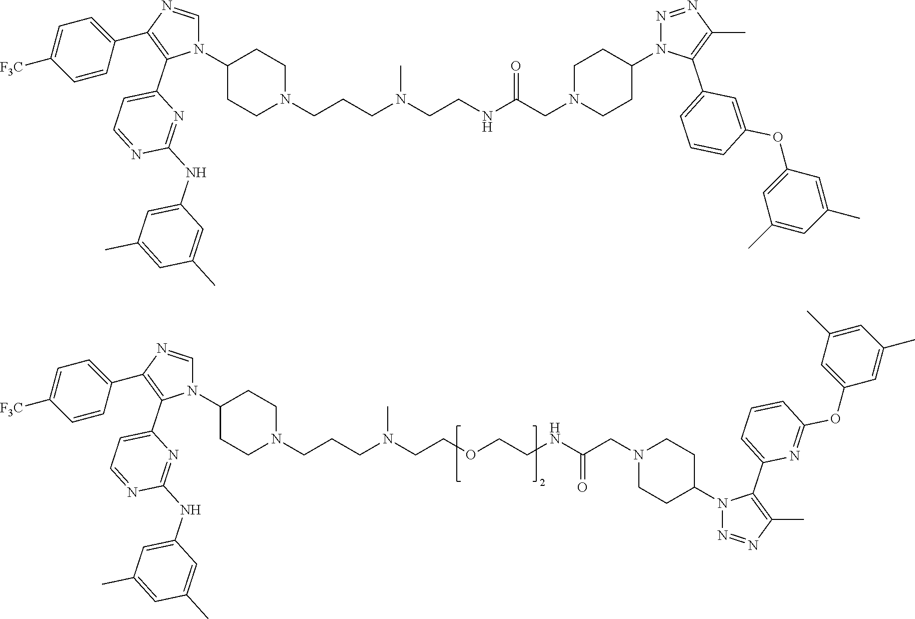

or a pharmaceutically acceptable salt thereof.

In some cases, an inhibitor of a BRD4 polypeptide can be as shown in Table 1.

TABLE 1
Inhibitors of BRD4 polypeptides.

| Name | Structure |
|---|---|
| UMN627^{a} aka 9-229 | 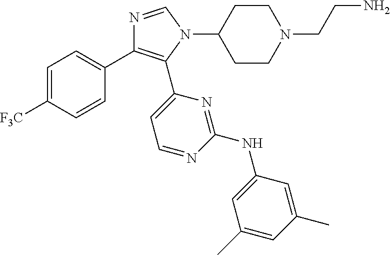 [SMILES: Cc1cc(C)cc(Nc2nccc(-c3c(-c4ccc(C(F)(F)F)cc4)ncn3C3CCN(CCN)CC3)n2)c1] |
| 9-73^{b} | 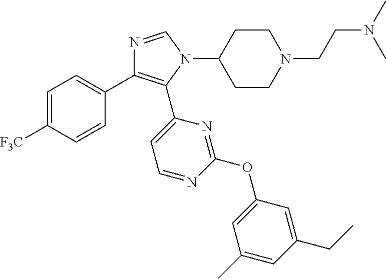 [SMILES: CCc1cc(C)cc(Oc2nccc(-c3c(-c4ccc(C(F)(F)F)cc4)ncn3C3CCN(CCN(C)C)CC3)n2)c1] |
| M-1-141^{c} | 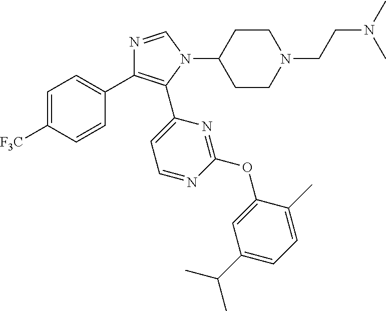 [SMILES: Cc1ccc(C(C)C)cc1Oc1nccc(-c2c(-c3ccc(C(F)(F)F)cc3)ncn2C2CCN(CCN(C)C)CC2)n1] |
| M-1-151^{c} | 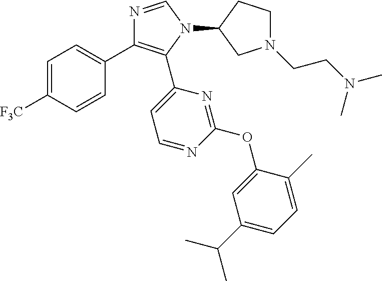 [SMILES: Cc1ccc(C(C)C)cc1Oc1nccc(-c2c(-c3ccc(C(F)(F)F)cc3)ncn2[C@H]2CCN(CCN(C)C)C2)n1] |
| 8-247^{d} | 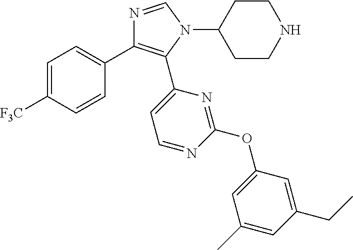 [SMILES: CCc1cc(C)cc(Oc2nccc(-c3c(-c4ccc(C(F)(F)F)cc4)ncn3C3CCNCC3)n2)c1] |
| 9-209^{c} | 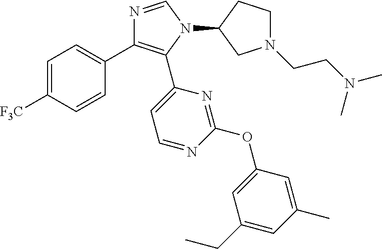 [SMILES: CCc1cc(C)cc(Oc2nccc(-c3c(-c4ccc(C(F)(F)F)cc4)ncn3[C@H]3CCN(CCN(C)C)C3)n2)c1] |
| AC4118^{c} | 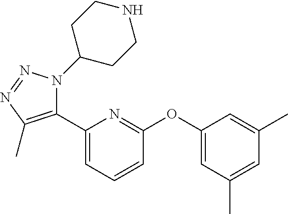 [SMILES: Cc1cc(C)cc(Oc2cccc(-c3c(C)nnn3C3CCNCC3)n2)c1] |
| AC6026^{c} | 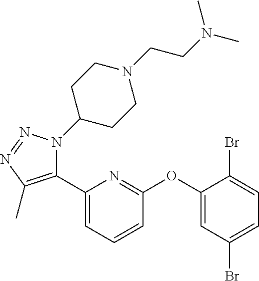 [SMILES: Cc1nnn(C2CCN(CCN(C)C)CC2)c1-c1cccc(Oc2cc(Br)ccc2Br)n1] |
| AC6027^{c} | 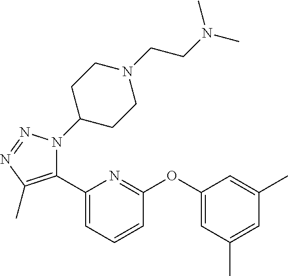 [SMILES: Cc1cc(C)cc(Oc2cccc(-c3c(C)nnn3C3CCN(CCN(C)C)CC3)n2)c1] |
| 9-169^{f} | 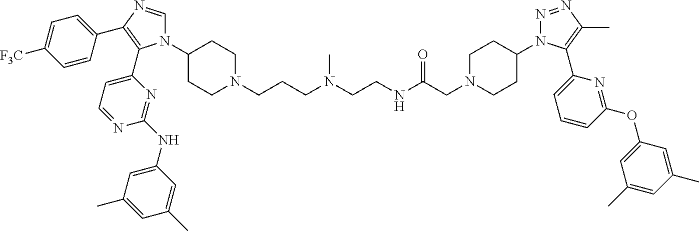 [SMILES: Cc1cc(C)cc(Nc2nccc(-c3c(-c4ccc(C(F)(F)F)cc4)ncn3C3CCN(CCCN(C)CCNC(=O)CN4CCC(n5nnc(C)c5-c5cccc(Oc6cc(C)cc(C)c6)n5)CC4)CC3)n2)c1] |
| 9-201^{g} | 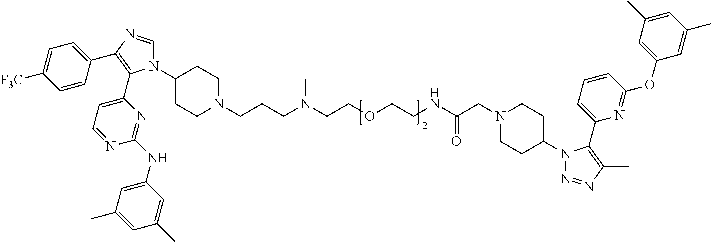 |
| OTX-015 | 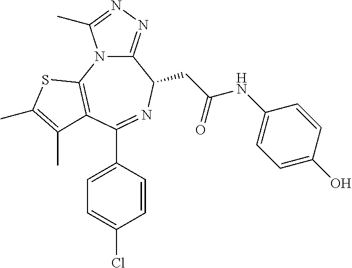 [SMILES: Cc1sc2c(c1C)C(c1ccc(Cl)cc1)=N[C@@H](CC(=O)Nc1ccc(O)cc1)c1nnc(C)n1-2] |
| JQ1 | 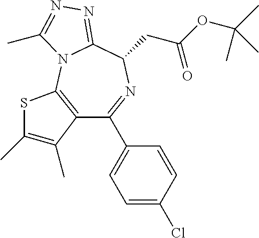 [SMILES: Cc1sc2c(c1C)C(c1ccc(Cl)cc1)=N[C@@H](CC(=O)OC(C)(C)C)c1nnc(C)n1-2] |
| MS436 | 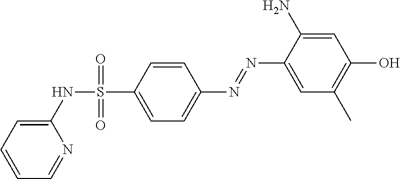 [SMILES: Cc1cc(/N=N/c2ccc(S(=O)(=O)Nc3ccccn3)cc2)c(N)cc1O] |
| ABBV- 075 | 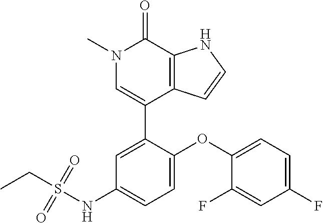 [SMILES: CCS(=O)(=O)Nc1ccc(Oc2ccc(F)cc2F)c(-c2cn(C)c(=O)c3[nH]ccc23)c1] |

^{a}The compound was prepared and tested as 4 × TFA, 6 × HCI, and 2 × HCl salt.
^{b}The compound was prepared and tested as a 3 × TFA salt.
^{c}The compound was prepared and tested as a 2 × TFA salt.
^{d}The compound was prepared and tested as a 4 × TFA salt.
^{e}The compound was prepared and tested as a TFA salt.
^{f}The compound was prepared and tested as a 6 × TFA salt.
^{g}The compound was prepared and tested as a 5 × TFA salt.

When an inhibitor of BRD4 polypeptide activity that can be used to treat a liver disease as described herein having the structure of Formula (I), Formula (II), or Formula (III) is a pharmaceutically acceptable salt, the pharmaceutically acceptable salt can be any pharmaceutically acceptable salt. A salt of a compound disclosed herein is formed between an acid and a basic group of the compound, such as an amino functional group, or a base and an acidic group of the compound, such as a carboxyl functional group. According to another embodiment, the compound is a pharmaceutically acceptable acid addition salt.

In some embodiments, acids commonly employed to form pharmaceutically acceptable salts of the compounds disclosed herein include inorganic acids such as hydrogen bisulfide, hydrochloric acid, hydrobromic acid, hydroiodic acid, sulfuric acid and phosphoric acid, as well as organic acids such as para-toluenesulfonic acid, salicylic acid, tartaric acid, bitartaric acid, ascorbic acid, maleic acid, besylic acid, fumaric acid, gluconic acid, glucuronic acid, formic acid, glutamic acid, methanesulfonic acid, ethanesulfonic acid, benzenesulfonic acid, lactic acid, oxalic acid, para-bromophenylsulfonic acid, carbonic acid, succinic acid, citric acid, benzoic acid and acetic acid, as well as related inorganic and organic acids. Such pharmaceutically acceptable salts thus include sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, terephthalate, sulfonate, xylene sulfonate, phenylacetate, phenylpropionate, phenylbutyrate, citrate, lactate, β-hydroxybutyrate, glycolate, maleate, tartrate, methanesulfonate, propanesulfonate, naphthalene-1-sulfonate, naphthalene-2-sulfonate, mandelate and other salts. In one embodiment, pharmaceutically acceptable acid addition salts include those formed with mineral acids such as hydrochloric acid and hydrobromic acid, and especially those formed with organic acids such as maleic acid.

In some embodiments, bases commonly employed to form pharmaceutically acceptable salts of the compounds disclosed herein include hydroxides of alkali metals, including sodium, potassium, and lithium; hydroxides of alkaline earth metals such as calcium and magnesium; hydroxides of other metals, such as aluminum and zinc; ammonia, organic amines such as unsubstituted or hydroxyl-substituted mono-, di-, or tri-alkylamines, dicyclohexylamine; tributyl amine; pyridine; N-methyl, N-ethylamine; diethylamine; triethylamine; mono-, bis-, or tris-(2—OH—(C1-C6)-alkylamine), such as N,N-dimethyl-N-(2-hydroxyethyl)amine or tri-(2-hydroxyethyl) amine; N-methyl-D-glucamine; morpholine; thiomorpholine; piperidine; pyrrolidine; and amino acids such as arginine, lysine, and the like.

In some embodiments, the compounds of the present disclosure, or pharmaceutically acceptable salts thereof, are substantially isolated.

At various places in the present specification, substituents of compounds of the invention are disclosed in groups or in ranges. It is specifically intended that the invention include each and every individual subcombination of the members of such groups and ranges. For example, the term “C₁₋₆ alkyl” is specifically intended to individually disclose methyl, ethyl, C₃ alkyl, C₄ alkyl, C₅ alkyl, and C₆ alkyl.

At various places in the present specification various aryl, heteroaryl, cycloalkyl, and heterocycloalkyl rings are described. Unless otherwise specified, these rings can be attached to the rest of the molecule at any ring member as permitted by valency. For example, the term “a pyridine ring” or “pyridinyl” may refer to a pyridin-2-yl, pyridin-3-yl, or pyridin-4-yl ring.

It is further appreciated that certain features of the invention, which are, for clarity, described in the context of separate embodiments, can also be provided in combination in a single embodiment. Conversely, various features of the invention which are, for brevity, described in the context of a single embodiment, can also be provided separately or in any suitable subcombination.

The term “n-membered” where n is an integer typically describes the number of ring-forming atoms in a moiety where the number of ring-forming atoms is n. For example, piperidinyl is an example of a 6-membered heterocycloalkyl ring, pyrazolyl is an example of a 5-membered heteroaryl ring, pyridyl is an example of a 6-membered heteroaryl ring, and 1,2,3,4-tetrahydro-naphthalene is an example of a 10-membered cycloalkyl group.

As used herein, the phrase “optionally substituted” means unsubstituted or substituted. The substituents are independently selected, and substitution may be at any chemically accessible position. As used herein, the term “substituted” means that a hydrogen atom is removed and replaced by a substituent. A single divalent substituent, e.g., oxo, can replace two hydrogen atoms. It is to be understood that substitution at a given atom is limited by valency.

Throughout the definitions, the term “Cₙ₋ₘ” indicates a range which includes the endpoints, wherein n and m are integers and indicate the number of carbons. Examples include C₁₋₄, C₁₋₆, and the like.

As used herein, the term “Cn-m alkyl”, employed alone or in combination with other terms, refers to a saturated hydrocarbon group that may be straight-chain or branched, having n to m carbons. Examples of alkyl moieties include, but are not limited to, chemical groups such as methyl, ethyl, n-propyl, isopropyl, n-butyl, tert-butyl, isobutyl, sec-butyl; higher homologs such as 2-methyl-1-butyl, n-pentyl, 3-pentyl, n-hexyl, 1,2,2-trimethylpropyl, and the like. In some embodiments, the alkyl group contains from 1 to 6 carbon atoms, from 1 to 4 carbon atoms, from 1 to 3 carbon atoms, or 1 to 2 carbon atoms

As used herein, the term “Cₙ₋ₘ haloalkyl”, employed alone or in combination with other terms, refers to an alkyl group having from one halogen atom to 2s+1 halogen atoms which may be the same or different, where “s” is the number of carbon atoms in the alkyl group, wherein the alkyl group has n to m carbon atoms. In some embodiments, the haloalkyl group is fluorinated only. In some embodiments, the alkyl group has 1 to 6, 1 to 4, or 1 to 3 carbon atoms.

As used herein, “Cₙ₋ₘ alkenyl” refers to an alkyl group having one or more double carbon-carbon bonds and having n to m carbons. Example alkenyl groups include, but are not limited to, ethenyl, n-propenyl, isopropenyl, n-butenyl, sec-butenyl, and the like. In some embodiments, the alkenyl moiety contains 2 to 6, 2 to 4, or 2 to 3 carbon atoms.

As used herein, “Cₙ₋ₘ alkynyl” refers to an alkyl group having one or more triple carbon-carbon bonds and having n to m carbons. Example alkynyl groups include, but are not limited to, ethynyl, propyn-1-yl, propyn-2-yl, and the like. In some embodiments, the alkynyl moiety contains 2 to 6, 2 to 4, or 2 to 3 carbon atoms.

As used herein, the term “Cₙ₋ₘ alkylene”, employed alone or in combination with other terms, refers to a divalent alkyl linking group having n to m carbons. Examples of alkylene groups include, but are not limited to, ethan-1,1-diyl, ethan-1,2-diyl, propan-1,1,-diyl, propan-1,3-diyl, propan-1,2-diyl, butan-1,4-diyl, butan-1,3-diyl, butan-1,2-diyl, 2-methyl-propan-1,3-diyl, and the like. In some embodiments, the alkylene moiety contains 2 to 6, 2 to 4, 2 to 3, 1 to 6, 1 to 4, or 1 to 2 carbon atoms.

As used herein, the term “Cₙ₋ₘ alkoxy”, employed alone or in combination with other terms, refers to a group of formula —O-alkyl, wherein the alkyl group has n to m carbons. Example alkoxy groups include, but are not limited to, methoxy, ethoxy, propoxy (e.g., n-propoxy and isopropoxy), butoxy (e.g., n-butoxy and tert-butoxy), and the like. In some embodiments, the alkyl group has 1 to 6, 1 to 4, or 1 to 3 carbon atoms.

As used herein, “Cₙ₋ₘ haloalkoxy” refers to a group of formula —O-haloalkyl having n to m carbon atoms. An example haloalkoxy group is OCF₃. In some embodiments, the haloalkoxy group is fluorinated only. In some embodiments, the alkyl group has 1 to 6, 1 to 4, or 1 to 3 carbon atoms.

As used herein, the term “amino” refers to a group of formula —NH₂.

As used herein, the term “Cₙ₋ₘ alkylamino” refers to a group of formula —NH (alkyl), wherein the alkyl group has n to m carbon atoms. In some embodiments, the alkyl group has 1 to 6, 1 to 4, or 1 to 3 carbon atoms. Examples of alkylamino groups include, but are not limited to, N-methylamino, N-ethylamino, N-propylamino (e.g., N-(n-propyl)amino and N-isopropylamino), N-butylamino (e.g., N-(n-butyl)amino and N-(tert-butyl)amino), and the like.

As used herein, the term “di(Cₙ₋ₘ-alkyl)amino” refers to a group of formula —N(alkyl)₂, wherein the two alkyl groups each has, independently, n to m carbon atoms. In some embodiments, each alkyl group independently has 1 to 6, 1 to 4, or 1 to 3 carbon atoms.

As used herein, the term “carboxy” refers to a —C(O)OH group.

As used herein, “halo” refers to F, Cl, Br, or I. In some embodiments, a halo is F, Cl, or Br.

As used herein, “cycloalkyl” refers to non-aromatic cyclic hydrocarbons including cyclized alkyl and/or alkenyl groups. Cycloalkyl groups can include mono- or polycyclic (e.g., having 2, 3 or 4 fused rings) groups and spirocycles. Ring-forming carbon atoms of a cycloalkyl group can be optionally substituted by 1 or 2 independently selected oxo or sulfide groups (e.g., C(O) or C(S)). Also included in the definition of cycloalkyl are moieties that have one or more aromatic rings fused (i.e., having a bond in common with) to the cycloalkyl ring, for example, benzo or thienyl derivatives of cyclopentane, cyclohexane, and the like. A cycloalkyl group containing a fused aromatic ring can be attached through any ring-forming atom including a ring-forming atom of the fused aromatic ring. Cycloalkyl groups can have 3, 4, 5, 6, 7, 8, 9, or 10 ring-forming carbons (C₃₋₁₀). In some embodiments, the cycloalkyl is a C₃₋₁₀ monocyclic or bicyclic cyclocalkyl. In some embodiments, the cycloalkyl is a C₃₋₇ monocyclic cyclocalkyl. Example cycloalkyl groups include cyclopropyl, cyclobutyl, cyclopentyl, cyclohexyl, cycloheptyl, cyclopentenyl, cyclohexenyl, cyclohexadienyl, cycloheptatrienyl, norbornyl, norpinyl, norcarnyl, adamantyl, and the like. In some embodiments, cycloalkyl is cyclopropyl, cyclobutyl, cyclopentyl, or cyclohexyl.

As used herein, “heterocycloalkyl” refers to non-aromatic monocyclic or polycyclic heterocycles having one or more ring-forming heteroatoms selected from O, N, or S. Included in heterocycloalkyl are monocyclic 4-, 5-, 6-, 7-, 8-, 9- or 10-membered heterocycloalkyl groups. Heterocycloalkyl groups can also include spirocycles. Example heterocycloalkyl groups include pyrrolidin-2-one, 1,3-isoxazolidin-2-one, pyranyl, tetrahydropuran, oxetanyl, azetidinyl, morpholino, thiomorpholino, piperazinyl, tetrahydrofuranyl, tetrahydrothienyl, piperidinyl, pyrrolidinyl, isoxazolidinyl, isothiazolidinyl, pyrazolidinyl, oxazolidinyl, thiazolidinyl, imidazolidinyl, azepanyl, benzazapene, and the like. Ring-forming carbon atoms and heteroatoms of a heterocycloalkyl group can be optionally substituted by 1 or 2 independently selected oxo or sulfido groups (e.g., C(O), S(O), C(S), or S(O)₂, etc.). The heterocycloalkyl group can be attached through a ring-forming carbon atom or a ring-forming heteroatom. In some embodiments, the heterocycloalkyl group contains 0 to 3 double bonds. In some embodiments, the heterocycloalkyl group contains 0 to 2 double bonds. Also included in the definition of heterocycloalkyl are moieties that have one or more aromatic rings fused (i.e., having a bond in common with) to the cycloalkyl ring, for example, benzo or thienyl derivatives of piperidine, morpholine, azepine, etc. A heterocycloalkyl group containing a fused aromatic ring can be attached through any ring-forming atom including a ring-forming atom of the fused aromatic ring. In some embodiments, the heterocycloalkyl is a monocyclic 4-6 membered heterocycloalkyl having 1 or 2 heteroatoms independently selected from nitrogen, oxygen, or sulfur and having one or more oxidized ring members. In some embodiments, the heterocycloalkyl is a monocyclic or bicyclic 4-10 membered heterocycloalkyl having 1, 2, 3, or 4 heteroatoms independently selected from nitrogen, oxygen, or sulfur and having one or more oxidized ring members.

The term “compound” as used herein is meant to include all stereoisomers, geometric isomers, tautomers, and isotopes of the structures depicted. Compounds herein identified by name or structure as one particular tautomeric form are intended to include other tautomeric forms unless otherwise specified.

The compounds described herein can be asymmetric (e.g., having one or more stereocenters). All stereoisomers, such as enantiomers and diastereomers, are intended unless otherwise indicated. Compounds of the present invention that contain asymmetrically substituted carbon atoms can be isolated in optically active or racemic forms. Methods on how to prepare optically active forms from optically inactive starting materials are known in the art, such as by resolution of racemic mixtures or by stereoselective synthesis. Many geometric isomers of olefins, C═N double bonds, N═N double bonds, and the like can also be present in the compounds described herein, and all such stable isomers are contemplated in the present invention. Cis and trans geometric isomers of the compounds of the present invention are described and may be isolated as a mixture of isomers or as separated isomeric forms. In some embodiments, the compound has the (R)-configuration. In some embodiments, the compound has the (S)-configuration.

Compounds provided herein also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Example prototropic tautomers include ketone—enol pairs, amide—imidic acid pairs, lactam—lactim pairs, enamine—imine pairs, and annular forms where a proton can occupy two or more positions of a heterocyclic system, for example, 1H- and 3H-imidazole, 1H-, 2H- and 4H-1,2,4-triazole, 1H- and 2H-isoindole, and 1H- and 2H-pyrazole. Tautomeric forms can be in equilibrium or sterically locked into one form by appropriate substitution.

In some cases, one or more inhibitors of a BRD4 polypeptide can be formulated into a composition (e.g., a pharmaceutically acceptable composition) for administration to a mammal (e.g., a human) having a liver disease (e.g., an ALD such as AH). For example, one or more inhibitors of a BRD4 polypeptide can be formulated together with one or more pharmaceutically acceptable carriers (additives), excipients, and/or diluents. Examples of pharmaceutically acceptable carriers, excipients, and diluents that can be used in a composition described herein include, without limitation, cyclodextrins (e.g., beta-cyclodextrins such as KLEPTOSE®), dimethylsulfoxide (DMSO), sucrose, lactose, starch (e.g., starch glycolate), cellulose, cellulose derivatives (e.g., modified celluloses such as microcrystalline cellulose, and cellulose ethers like hydroxypropyl cellulose (HPC) and cellulose ether hydroxypropyl methylcellulose (HPMC)), xylitol, sorbitol, mannitol, gelatin, polymers (e.g., polyvinylpyrrolidone (PVP), polyethylene glycol (PEG), crosslinked polyvinylpyrrolidone (crospovidone), carboxymethyl cellulose, polyethylene-polyoxypropylene-block polymers, and crosslinked sodium carboxymethyl cellulose (croscarmellose sodium)), titanium oxide, azo dyes, silica gel, fumed silica, talc, magnesium carbonate, vegetable stearin, magnesium stearate, aluminum stearate, stearic acid, antioxidants (e.g., vitamin A, vitamin E, vitamin C, retinyl palmitate, and selenium), citric acid, sodium citrate, parabens (e.g., methyl paraben and propyl paraben), petrolatum, dimethyl sulfoxide, mineral oil, serum proteins (e.g., human serum albumin), glycine, sorbic acid, potassium sorbate, water, salts or electrolytes (e.g., saline, protamine sulfate, disodium hydrogen phosphate, potassium hydrogen phosphate, sodium chloride, and zinc salts), colloidal silica, magnesium trisilicate, polyacrylates, waxes, wool fat, lecithin, and corn oil.

In some cases, when a composition containing one or more inhibitors of a BRD4 polypeptide is administered to a mammal (e.g., a human) having a liver disease (e.g., an ALD such as AH), the composition can be designed for oral or parenteral (including, without limitation, a subcutaneous, intramuscular, intravenous, intradermal, intra-cerebral, intrathecal, or intraperitoneal (i.p.) injection) administration to the mammal. Compositions suitable for oral administration include, without limitation, liquids, tablets, capsules, pills, powders, gels, and granules. In some cases, compositions suitable for oral administration can be in the form of a food supplement. In some cases, compositions suitable for oral administration can be in the form of a drink supplement. Compositions suitable for parenteral administration include, without limitation, aqueous and non-aqueous sterile injection solutions that can contain anti-oxidants, buffers, bacteriostats, and solutes that render the formulation isotonic with the blood of the intended recipient.

A composition containing one or more inhibitors of a BRD4 polypeptide can be administered to a mammal (e.g., a human) having a liver disease (e.g., an ALD such as AH) in any appropriate amount (e.g., any appropriate dose). An effective amount of a composition containing one or more inhibitors of a BRD4 polypeptide can be any amount that can treat a mammal having a liver disease (e.g., an ALD such as AH) as described herein without producing significant toxicity to the mammal. In cases where an inhibitor of a BRD4 polypeptide is UMN627, an effective amount of one or more inhibitors of a BRD4 polypeptide can be from about 5 milligrams per kilogram body weight (mg/kg) to about 30 mg/kg (e.g., from about 5 mg/kg to about 25 mg/kg, from about 5 mg/kg to about 20 mg/kg, from about 5 mg/kg to about 15 mg/kg, from about 5 mg/kg to about 10 mg/kg, from about 10 mg/kg to about 30 mg/kg, from about 15 mg/kg to about 30 mg/kg, from about 20 mg/kg to about 30 mg/kg, from about 25 mg/kg to about 30 mg/kg, from about 10 mg/kg to about 25 mg/kg, from about 15 mg/kg to about 20 mg/kg, from about 10 mg/kg to about 15 mg/kg, or from about 20 mg/kg to about 25 mg/kg). For example, an effective amount of a composition containing can include from about 20 mg/kg to about 25 mg/kg (e.g., 22 mg/kg) UMN627. The effective amount can remain constant or can be adjusted as a sliding scale or variable dose depending on the mammal's response to treatment. Various factors can influence the actual effective amount used for a particular application. For example, the frequency of administration, duration of treatment, use of multiple treatment agents, route of administration, and/or severity of the liver disease (e.g., an ALD such as AH) in the mammal being treated may require an increase or decrease in the actual effective amount administered.

A composition containing one or more inhibitors of a BRD4 polypeptide can be administered to a mammal (e.g., a human) having a liver disease (e.g., an ALD such as AH) in any appropriate frequency. The frequency of administration can be any frequency that can treat a mammal having a liver disease (e.g., an ALD such as AH) without producing significant toxicity to the mammal. For example, the frequency of administration can be from about once a day to about once a week, from about once a week to about once a month, or from about twice a month to about once a month. The frequency of administration can remain constant or can be variable during the duration of treatment. As with the effective amount, various factors can influence the actual frequency of administration used for a particular application. For example, the effective amount, duration of treatment, use of multiple treatment agents, and/or route of administration may require an increase or decrease in administration frequency.

A composition containing one or more inhibitors of a BRD4 polypeptide can be administered to a mammal (e.g., a human) having a liver disease (e.g., an ALD such as AH) for any appropriate duration. An effective duration for administering or using a composition containing one or more inhibitors of a BRD4 polypeptide can be any duration that can treat a mammal having a liver disease (e.g., an ALD such as AH) without producing significant toxicity to the mammal. For example, the effective duration can vary from several weeks to several months, from several months to several years, or from several years to a lifetime. Multiple factors can influence the actual effective duration used for a particular treatment. For example, an effective duration can vary with the frequency of administration, effective amount, use of multiple treatment agents, and/or route of administration.

In some cases, methods for treating a mammal (e.g., a human) having a liver disease (e.g., an ALD such as AH) can include administering to the mammal one or more inhibitors of a BRD4 polypeptide as the sole active ingredient to treat the mammal. For example, a composition containing one or more inhibitors of a BRD4 polypeptide can include the one or more inhibitors of a BRD4 polypeptide as the sole active ingredient in the composition that is effective to treat a mammal having a liver disease (e.g., an ALD such as AH).

In some cases, methods for treating a mammal (e.g., a human) having a liver disease (e.g., an ALD such as AH) as described herein (e.g., by administering one or more inhibitors of a BRD4 polypeptide) also can include administering to the mammal one or more (e.g., one, two, three, four, five or more) additional agents/therapies used to treat a liver disease (e.g., an ALD such as AH). For example, a combination therapy used to treat a liver disease (e.g., an ALD such as AH) can include administering to the mammal (e.g., a human) one or more inhibitors of a BRD4 polypeptide described herein and one or more (e.g., one, two, three, four, five or more) agents used to treat a liver disease. Examples of agents that can be administered to a mammal to treat a liver disease include, without limitation, nutritional supplements, corticosteroids (e.g., glucocorticoids such as prednisolone), pentoxifylline, antibiotics, inhibitors of other epigenetic polypeptides, and any combinations thereof. In cases where one or more inhibitors of a BRD4 polypeptide are used in combination with additional agents used to treat a liver disease, the one or more additional agents can be administered at the same time (e.g., in a single composition containing both one or more inhibitors of a BRD4 polypeptide and the one or more additional agents) or independently. For example, one or more inhibitors of a BRD4 polypeptide described herein can be administered first, and the one or more additional agents administered second, or vice versa.

In some cases, a combination therapy used to treat a liver disease (e.g., an ALD such as AH) can include administering to the mammal (e.g., a human) one or more inhibitors of a BRD4 polypeptide described herein and performing one or more (e.g., one, two, three, four, five or more) additional therapies used to treat a liver disease on the mammal. Examples of therapies used to treat a liver disease include, without limitation, alcohol cessation counseling and/or liver transplantation. In cases where one or more inhibitors of a BRD4 polypeptide described herein are used in combination with one or more additional therapies used to treat a liver disease, the one or more additional therapies can be performed at the same time or independently of the administration of one or more inhibitors of a BRD4 polypeptide described herein. For example, one or more inhibitors of a BRD4 polypeptide described herein can be administered before, during, or after the one or more additional therapies are performed.

The invention will be further described in the following examples, which do not limit the scope of the invention described in the claims.

### EXAMPLES

### Example 1: Super Enhancer Regulation of Cytokine-Induced Chemokine Production in Alcoholic Hepatitis

Alcoholic hepatitis (AH) is associated with liver neutrophil infiltration through activation of cytokine pathways such as TNFα signaling, leading to elevated chemokine expression.

This Example demonstrates upregulation of multiple CXCL chemokines in AH, and identifies liver sinusoidal endothelial cells (LSEC) as source of CXCL expression in the human liver. This Example also demonstrates that inhibition of bromodomain and extraterminal (BET) polypeptides can decrease neutrophil infiltration in AH.

### Results

### Gene Expression and Histone Modification in AH and Normal Livers

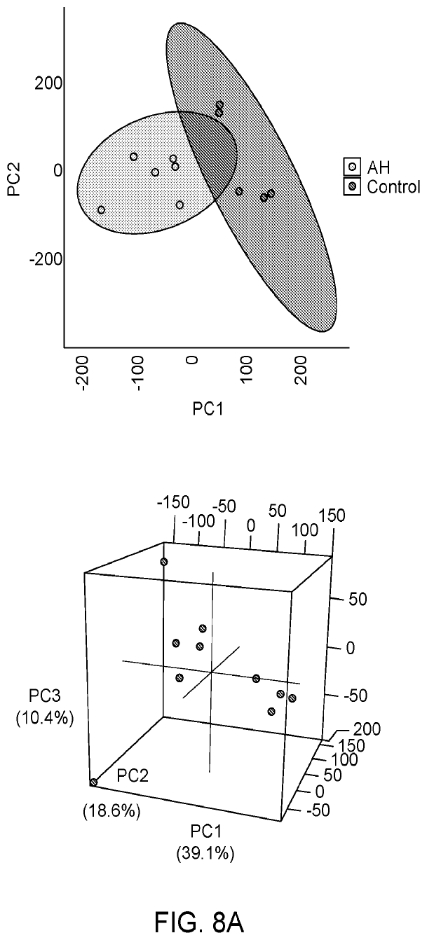

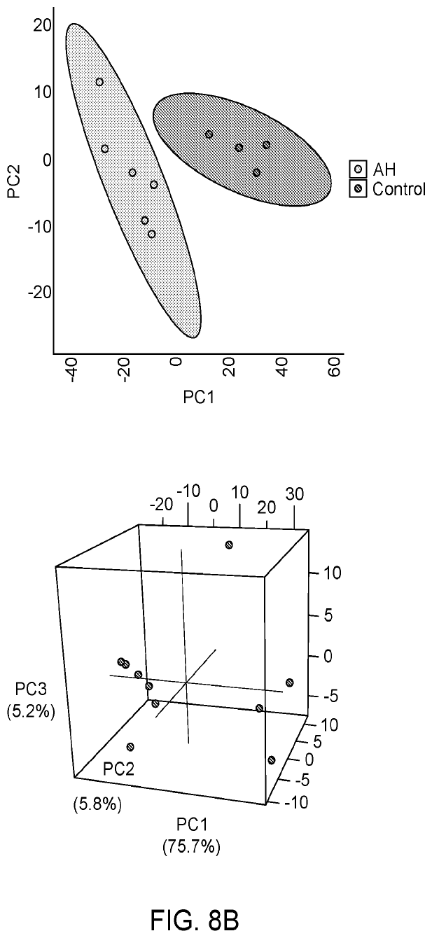

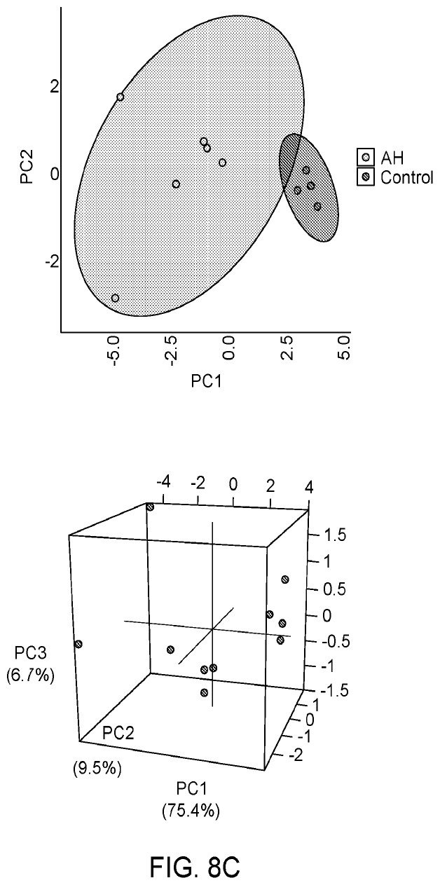

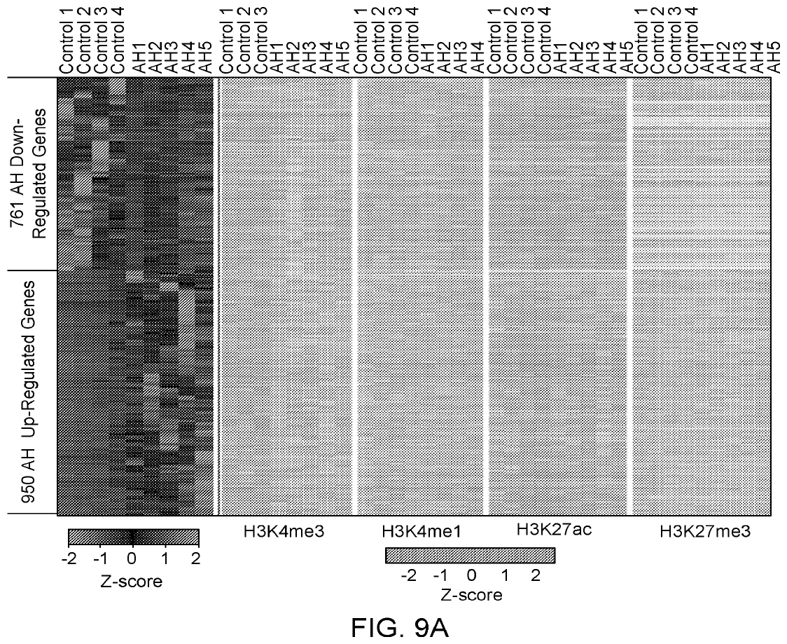

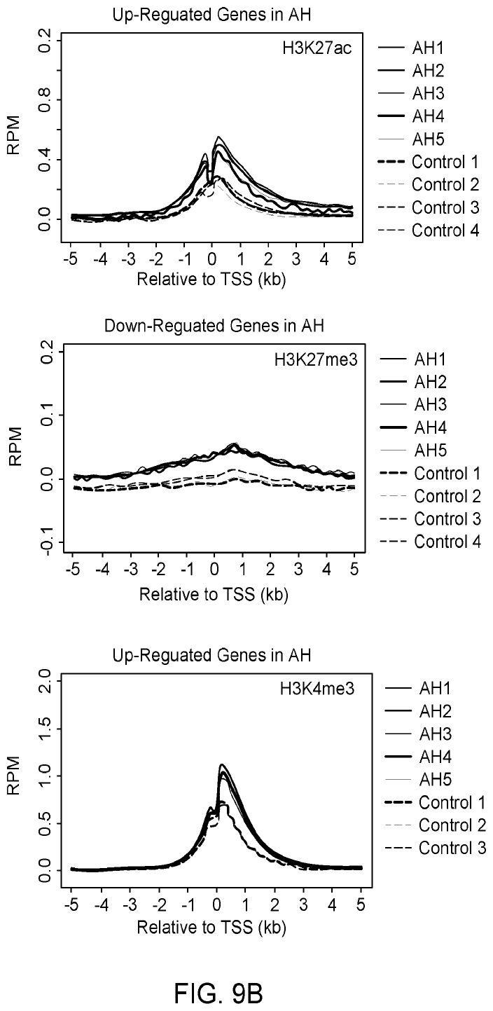

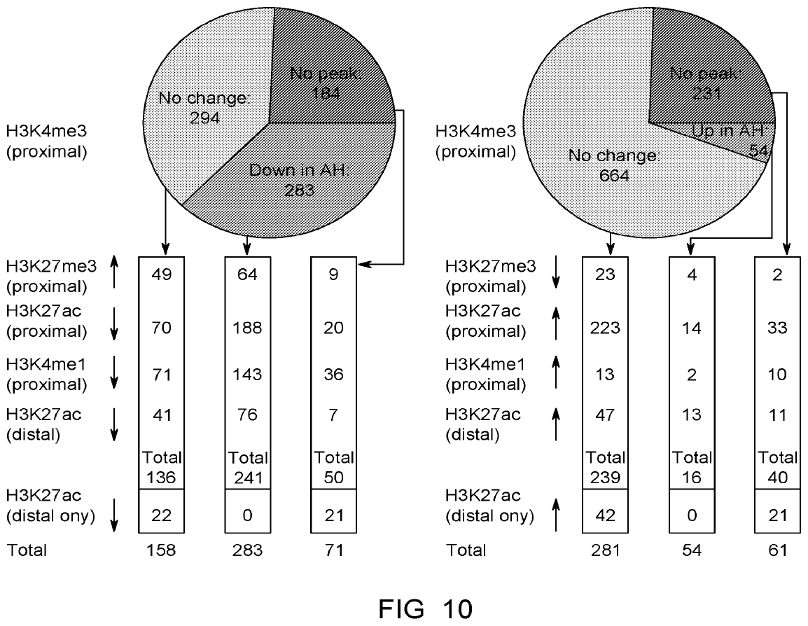

To characterize the gene expression changes in the livers of AH patients, liver explants were obtained at the time of liver transplantation from patients with severe AH and analyzed by RNA-Seq. Individuals with normal livers were used as controls. Demographics and clinical characteristics of AH subjects are summarized in FIG. 7. Principal Component Analysis (PCA) of RNA-seq data revealed transcriptomic alterations in AH livers (FIG. 8A). 950 genes were identified that are significantly upregulated and another 761 genes were identified that were significantly downregulated in AH livers (FDR<=0.01 and log2 (fold change)>=1.5, FIG. 9A). To understand whether epigenetic mechanisms play a role in the transcriptional dysregulation in AH, ChIP-Seq was performed on these samples for 4 histone modifications: H3K4me3 preferentially associated with promoters, H3K4me1 and H3K27ac preferentially associated with enhancers, and H3K27me3 associated with Polycomb repression. Peaks were mapped to the nearest transcription start site (TSS). Plotting the ChIP-Seq signal density over TSS±2 kb regions revealed that AH up-regulated genes are generally associated with increased signals of active marks (H3K4me3, H3K4me1 and H3K27ac) and decreased signal of H3K27me3, and a reverse trend was observed for AH down-regulated genes (FIG. 9A and FIG. 10). A similar pattern was revealed when plotting the aggregate histone mark signals (reads per million) over TSS±5 kb regions of AH up- and down-regulated genes (FIG. 9B). These findings suggest that histone modification patterns correlate with gene expression in a significant subset of genes dysregulated in AH and further raise the possibility that epigenetic mechanisms may play regulatory roles in the disease process.

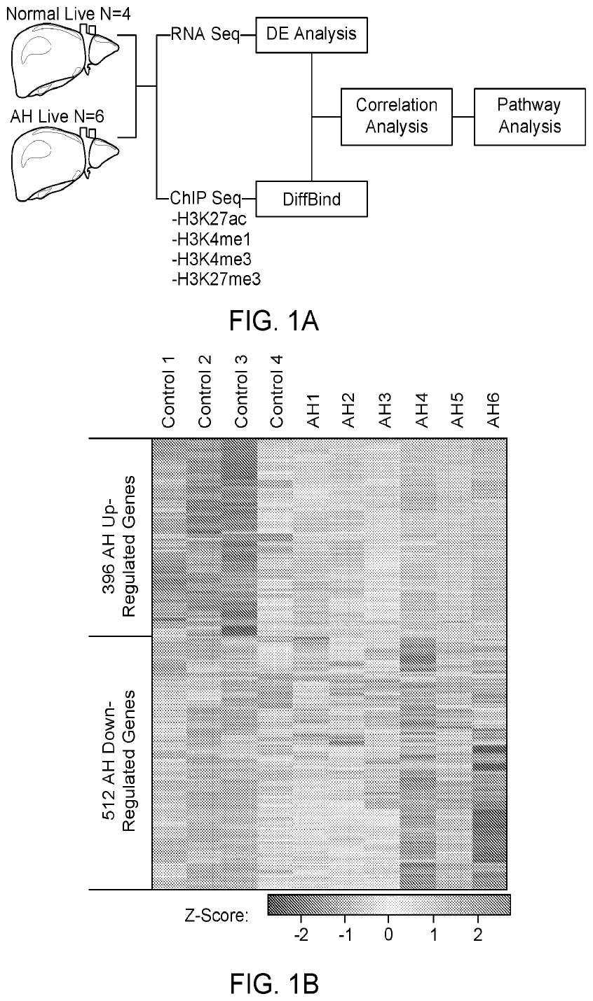

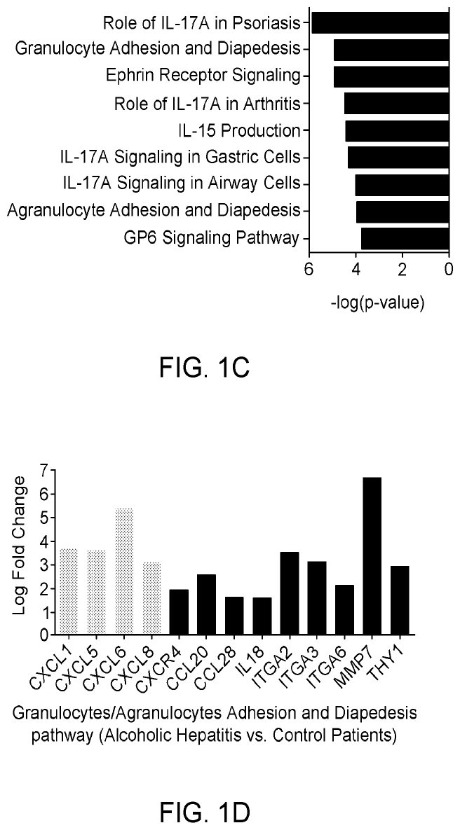

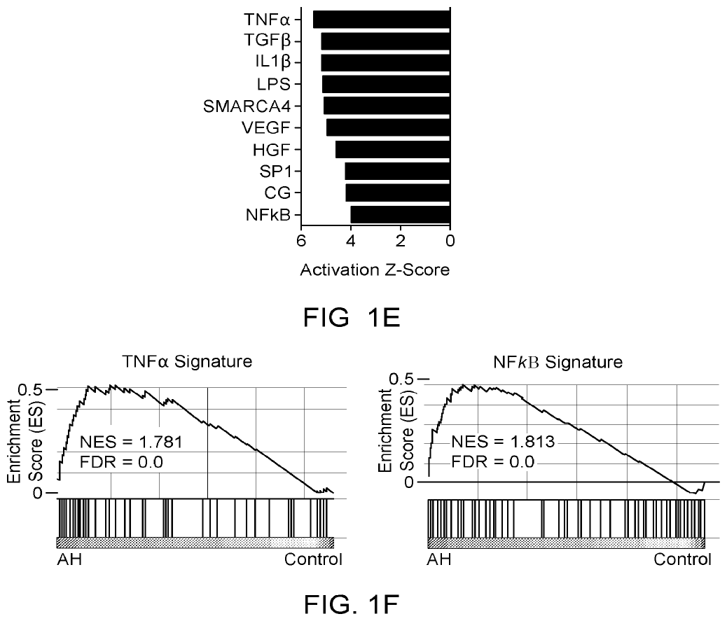

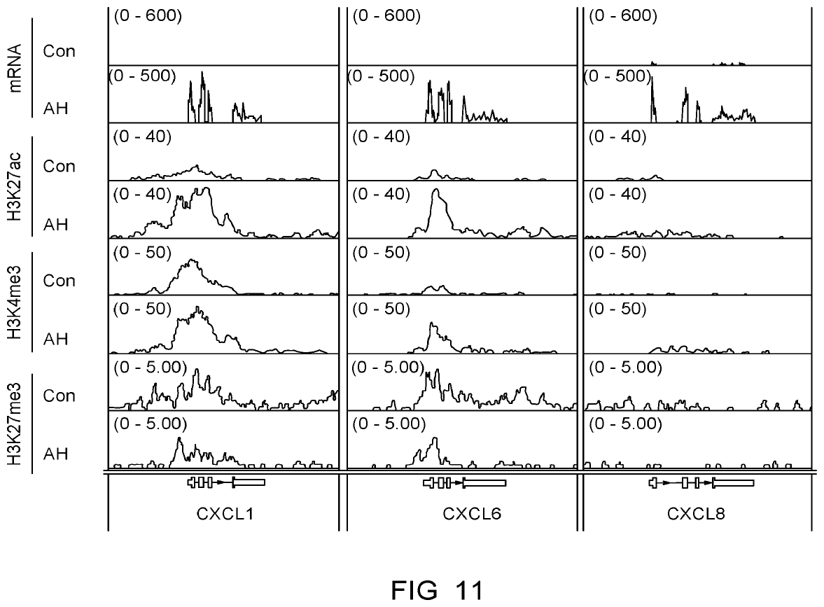

Integration of differential occupancy profiles of histone modifications with differential gene expression identified candidate genes that may be subjected to epigenetic regulation (schema shown in FIG. 1A). In this analysis, 396 of the 950 significantly upregulated genes and 512 of 761 significantly downregulated genes in AH livers also showed corresponding differential histone mark occupancy (FIG. 1B). Ingenuity Pathway Analysis (IPA) identified enrichment of numerous signaling pathway associated with differential expression and histone mark profiles in AH. The top affected pathways included the granulocyte adhesion and diapedesis pathway and the agranulocyte adhesion and diapedesis pathway, with the granulocyte adhesion and diapedesis pathway being the most second affected pathway when only the differentially upregulated genes with congruent histone modifications were examined (FIG. 1C). PCA of the differentially expressed genes with congruent histone modifications as well as the granulocyte/agranulocyte adhesion and diapedesis pathway associated genes showed excellent discrimination between AH and normal samples (FIG. 8B, 8C). Within the granulocyte/agranulocyte adhesion and diapedesis pathways, several CXCL chemokines were located near the same gene locus on chromosome 4 and showed remarkable upregulation in AH patients (FIG. 1D). Subsequent analysis focused on CXCL1, 6 and 8 due to their robust expression in the AH cohort and their role in immune cell chemotaxis, particularly neutrophils. These genes were enriched for activating modifications (H3K27ac and H3K4me3) and depleted for inhibitory mark H3K27me3 within both promoter regions and gene bodies in AH (FIG. 11). Given the central role of neutrophilic infiltration in the pathogenesis of AH, the upregulation of these CXCL genes provides a mechanistic link between local inflammation and systemic neutrophilic mobilization in the development of AH. To study the regulatory mechanism driving gene expression reprogramming in AH, upstream regulator analysis was performed and identified multiple pathways well-studied in liver inflammatory signaling, including TNFα, TGFβ, IL1β, among others (FIG. 1E). Gene set enrichment analysis (GSEA) of TNFα and NF-κB pathway target genes showed selective upregulation in AH patients (FIG. 1F). Upstream regulator analysis also highlighted multiple epigenetic modifiers, including SMARCA4 and SP1, that are activated in AH (FIG. 1E).

### Liver Sinusoidal Endothelial Cells and CXCL Chemokines in AH

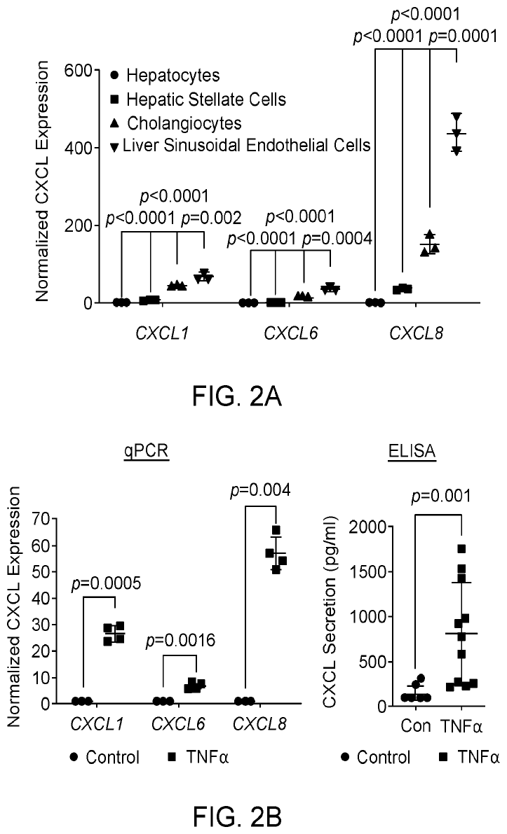

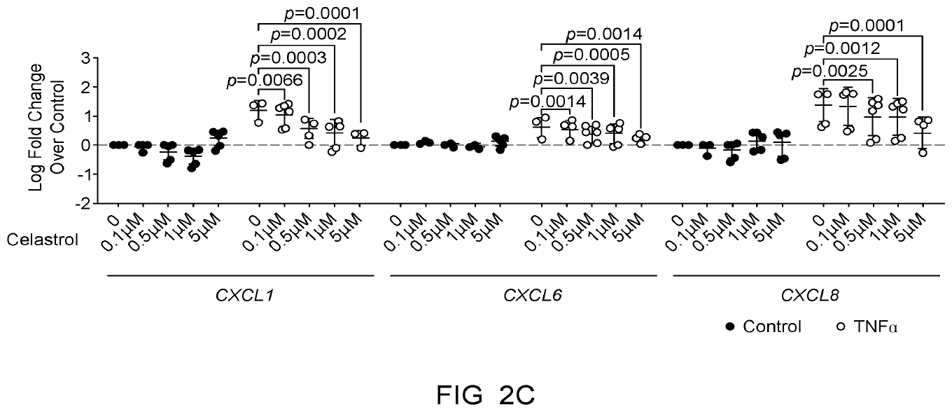

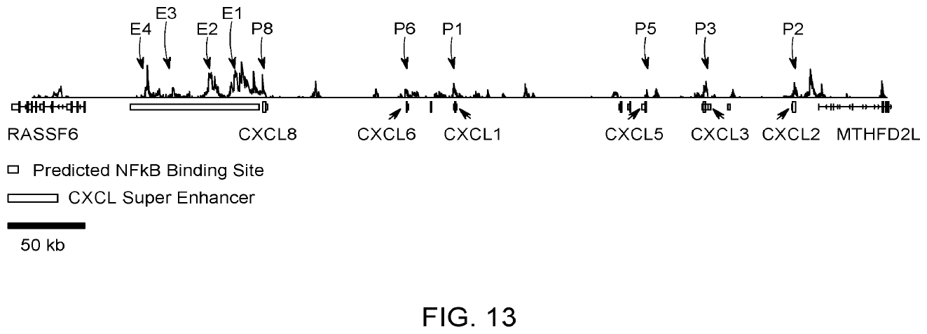

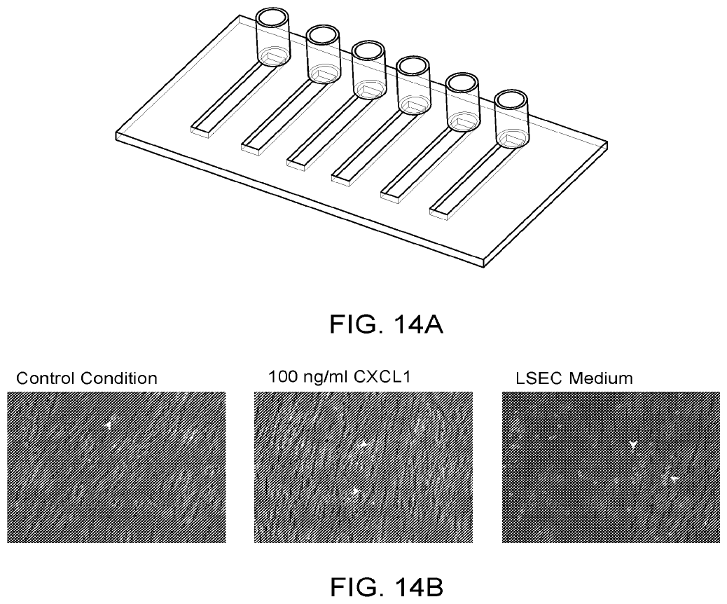

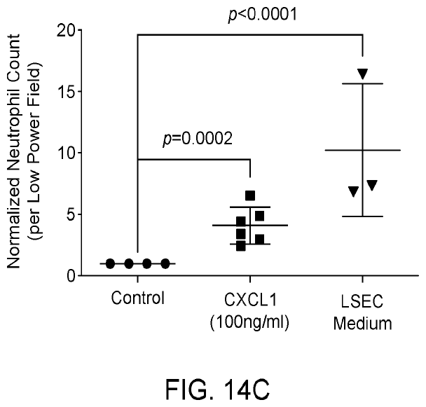

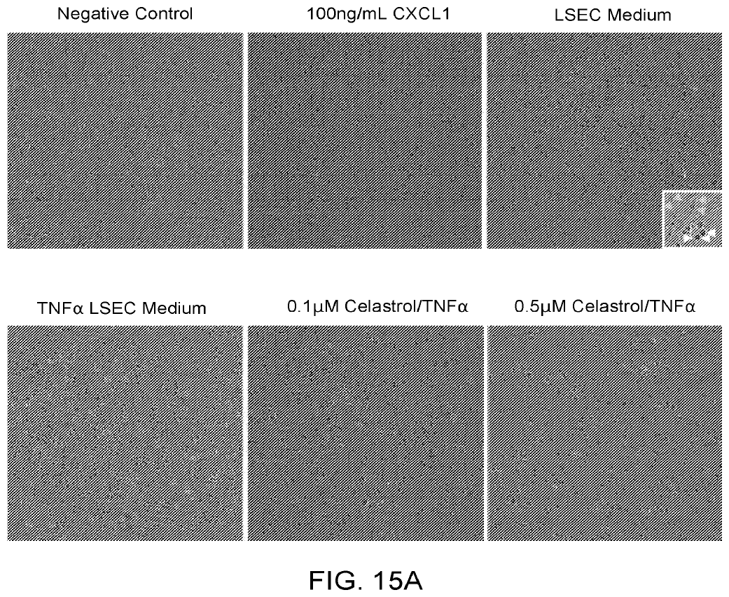

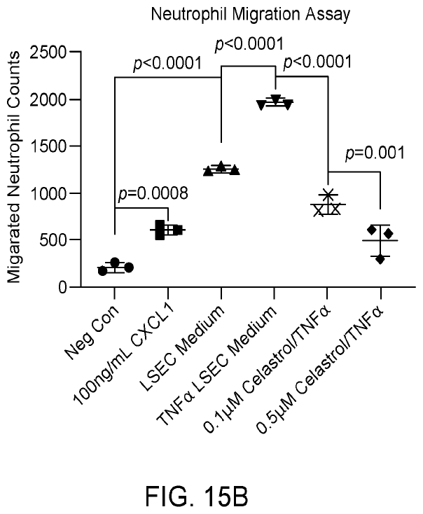

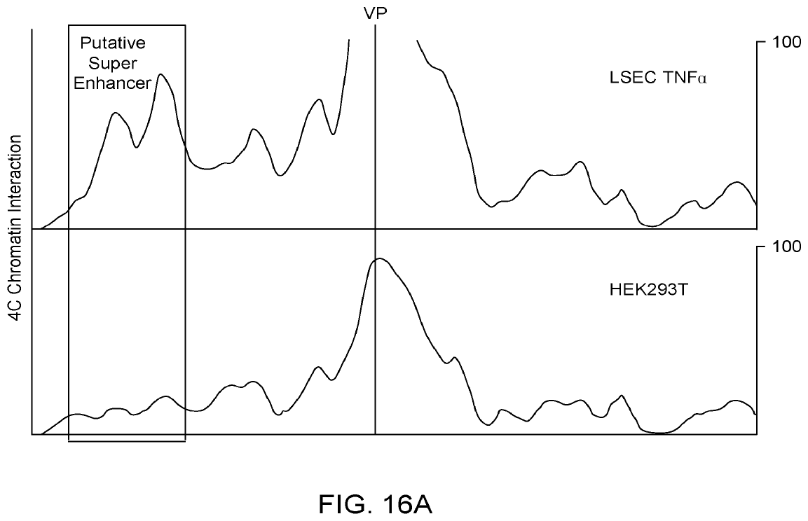

Coincidence of mutually exclusive marks H3K27ac and H3K27me3 over the same gene promoters (FIG. 11) suggested concordant epigenetic events occurring in different cells and, possibly, in different cell types. To identify the cellular source of elevated liver CXCL chemokine production in AH, RNA-Seq analysis was performed in several primary human liver cell types, including LSECs, intrahepatic biliary epithelial cells (cholangiocytes), and hepatic stellate cells, as well as hepatocyte cell-line HepG2, and LSECs were found to have the highest expression of CXCL1, 6, and 8 (FIG. 2A). As TNFα is a well-known activator of chemotaxis and was identified as a key upstream regulator of differentially expressed genes in the earlier pathway analysis (FIG. 1E), the LSEC response to TNFα simulation was examined. LSECs demonstrated a significant increase in the expression of CXCL genes upon exposure to TNFα in vitro (FIG. 2B). NF-κB was another key signaling intermediary in the upstream regulator analysis (FIG. 1E). Using the transcription factor binding profiles curated in the JASPAR database, consensus NF-κB binding sites (>95% consensus) in the promoter region of each CXCL chemokine were identified (FIG. 13). To test NF-κB signaling in LSECs, celastrol, a proteasome inhibitor that blocks NF-κB transport to the nucleus, was applied to LSECs, which significantly decreased CXCL chemokine expression and abrogated the stimulatory effect of TNFα stimulation in a dose-dependent manner (FIG. 2C). These findings suggest that LSEC production of CXCL chemokines is NF-κB dependent, and that TNFα augments CXCL expression through the action of NF-κB. To simulate the process of neutrophil recruitment in vitro, a LSEC-coated microfluidic system and a transwell two chamber system was utilized to assess the effect of CXCL chemokines on neutrophil adhesion and chemotaxis. Recombinant CXCL1 increased neutrophil adhesion. Conditioned medium from LSECs increased neutrophil attachment compared to fresh medium (FIG. 14). This chemotactic effect was further accentuated by TNFα pretreatment of LSECs and decreased with pretreatment with celastrol, a pharmacologic inhibitor of NF-κB translation, suggesting that CXCL differential expression has biologic relevance (FIG. 15).

### Super Enhancer-induced Expression of Multiple CXCL Chemokines is NF-κB-Dependent

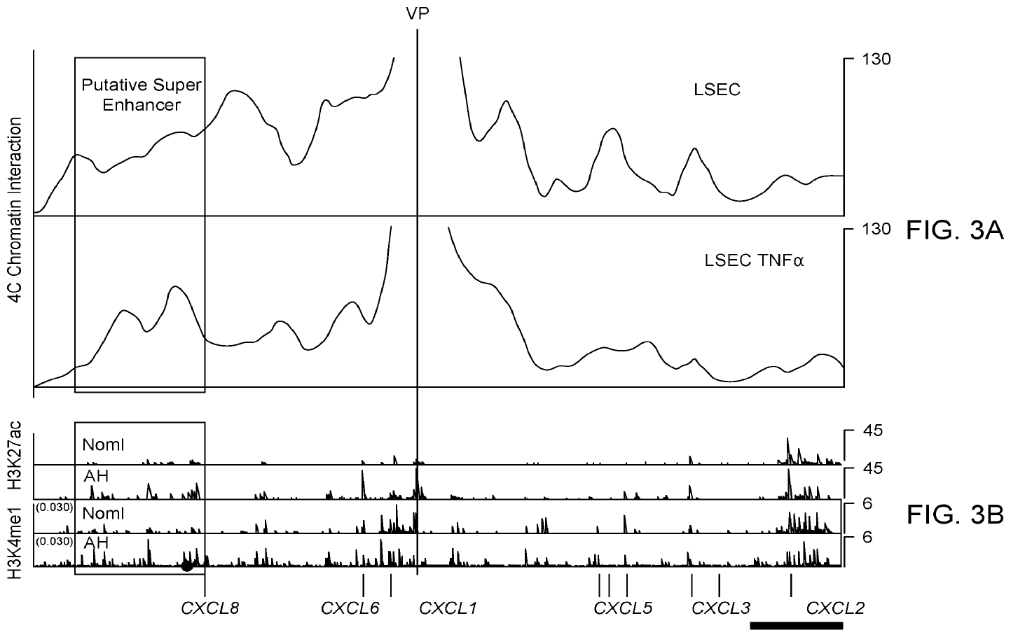

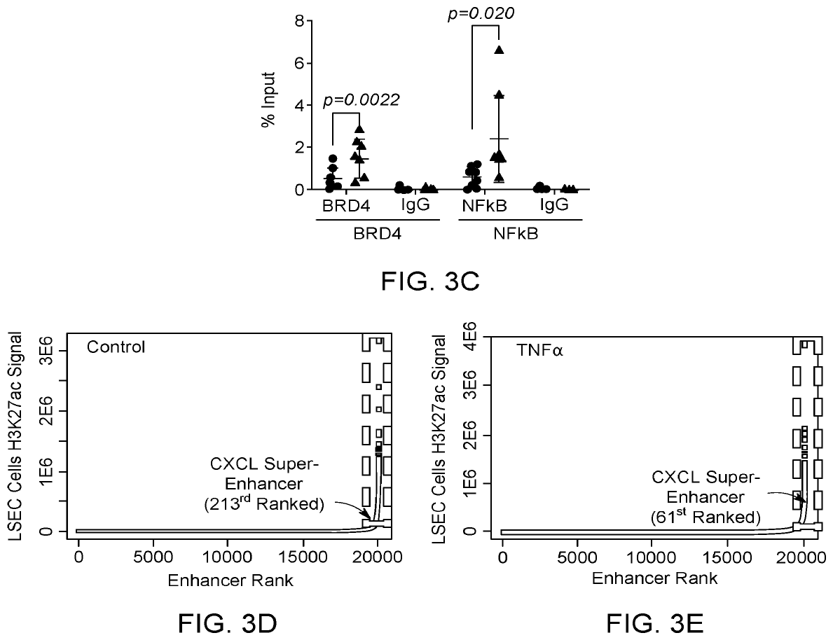

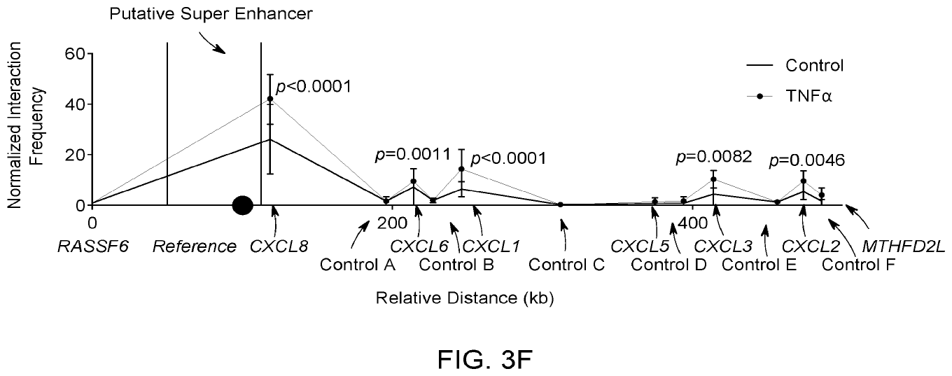

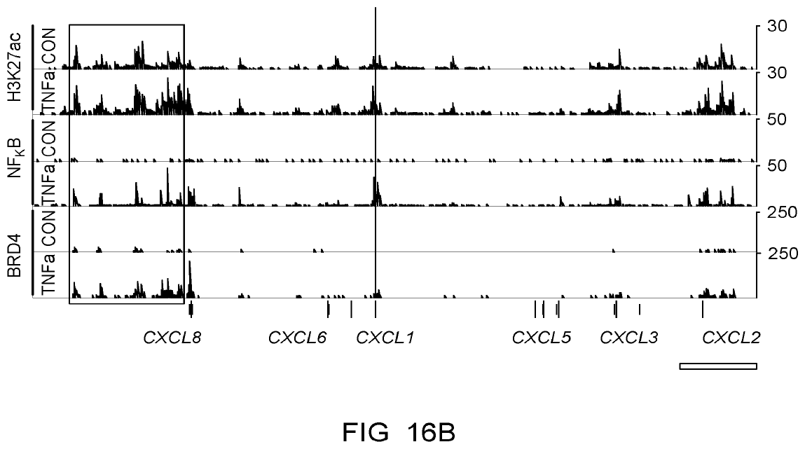

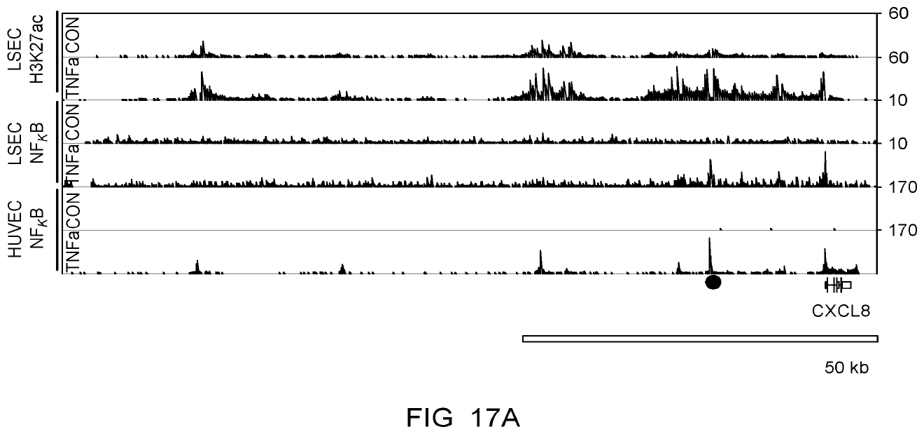

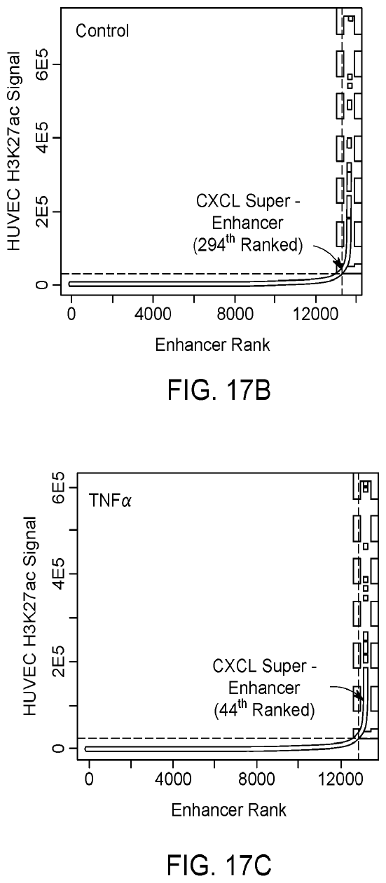

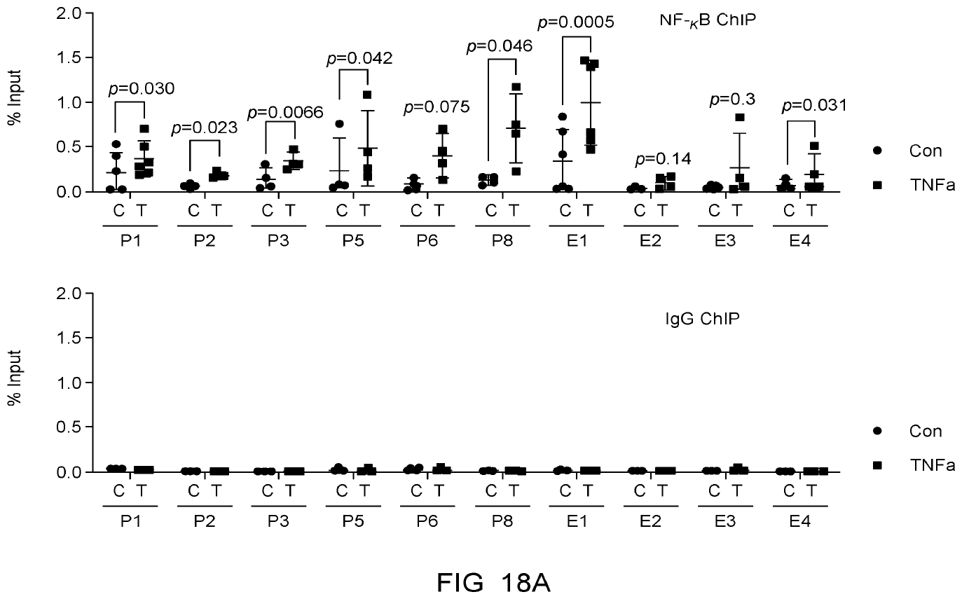

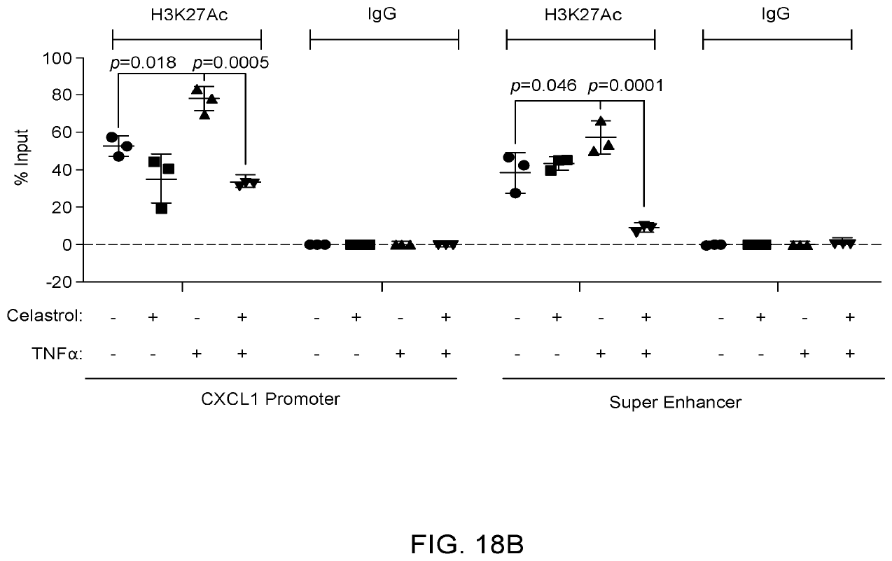

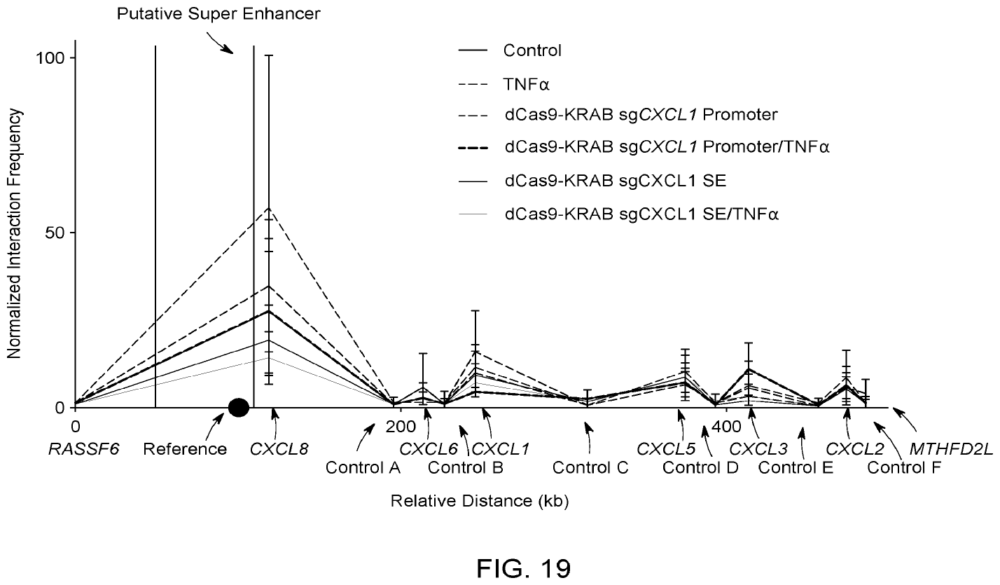

To identify the presence of a super enhancer regulating the expression of chemokines CXCL1, 6, and 8 under the control of TNFα as a putative CXCL master regulatory element in LSEC cells, circular chromosome conformation capture-sequencing (4C-Seq) was utilized. The CXCL1 promoter was used as the “view point” and the 3-dimensional interactions occurring genome-wide with this region were investigated in the presence and absence of TNFα stimulation. A 75 kb region proximal to the CXCL8 gene locus was highly associated with the CXCL1 promoter following TNFα stimulation, particularly in response to TNFα (FIG. 3A). In AH livers, this gene locus showed increased occupancy for H3K27ac and H3K4me1, which together define active enhancer sites, in response to TNFα (FIG. 3B). In contrast, the same site showed reduced interactions with the CXCL1 promoter in HEK293T cells which express CXCL minimally (FIG. 16A). To further study the role of NF-κB in LSEC CXCL expression, NF-κB (RELA subunit) ChIP-Seq datasets in HUVEC cells (GSE53998) were utilized, and NF-κB binding in the CXCL locus was analyzed. Following TNFα stimulation, multiple strong NF-κB binding peaks were found in the putative enhancer region as well as in the promoter regions of CXCL genes, showing NF-κB binding motif curated in the JASPAR database (FIG. 16B). NF-κB (RELA/p65) ChIPseq was performed in LSECs and a similar pattern of enhanced NF-κB binding with TNFα stimulation was observed (FIG. 17A). ChIP-qPCR experiments further confirmed these results, and NF-κB binding was increased in the promoters of multiple CXCL genes in response to TNFα stimulation (FIG. 18A). BRD4 binding was strongly enriched in the putative CXCL enhancer region in HUVEC cells after TNFα stimulation, largely overlapping with NF-κB peaks (FIG. 16B). Binding of NF-κB to the predicted binding sites in the putative enhancer region in LSEC cells was examined. These sites were selected based on the enrichment of H3K27ac ChIP-Seq signal in AH livers as well as strong NF-κB binding in HUVEC and LSEC cells (FIG. 16B and FIG. 17A). The site with the highest NF-κB ChIPseq signal also showed the most TNFα responsiveness in ChIP-qPCR (FIG. 17A and FIG. 18A). Both NF-κB and BRD4 bound specifically and robustly to this site after TNFα stimulation (FIG. 3C). Given the large size of this putative enhancer region and high occupancy of the transcriptional coactivator BRD4, the interaction site identified through 4C-Seq may be a super enhancer with CXCL1 as one of its target genes in LSECs. H3K27ac ChIPseq was performed on LSEC cells with and without TNFα stimulation. H3K27ac occupancy over the putative CXCL super enhancer was increased with TNFα treatment. H3K27ac ChIPseq was analyzed using the Rank Ordering of Super Enhancer (ROSE) algorithm and this site was identified as a super enhancer in LSEC cells both in the presence and absence of TNFα, but its site ranking rose higher under TNFα stimulation (FIG. 3D, 3E). ROSE analysis of HUVEC H3K27ac ChIP-Seq data (GSE53998) similarly identified this putative enhancer as a super enhancer in HUVEC cells (FIG. 17B). To explore if this super enhancer also regulates other CXCL genes at this gene locus in LSEC, a targeted chromosome conformation capture (3C) experiment was performed to examine the chromatin interaction between this super enhancer with the promoters of CXCL1, 2, 3, 5, 6, or 8 genes. Sequences from a nearby non-inflammatory gene, RASSF6, as well as intronic segments between CXCL genes were used as negative controls (FIG. 3F). The previously selected NF-κB binding site on the super enhancer was chosen as the reference site to assess for interaction with various promoters. Increased interaction was found between the super enhancer with promoters of CXCL1, 2, 3, 6, and 8 which increased with TNFα stimulation (FIG. 19). CXCL5 did not show increased interaction with the putative super enhancer. Collectively, these chromatin binding and interaction experiments identified a super enhancer with multiple CXCL chemokines as putative target genes in LSECs.

### Epigenetic Suppression of the CXCL Super Enhancer and CXCL1 Promoter Sites Modulate Chemokine Gene Expression

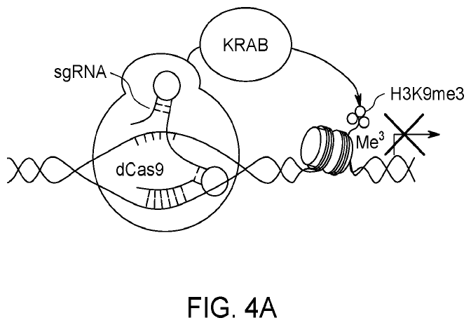

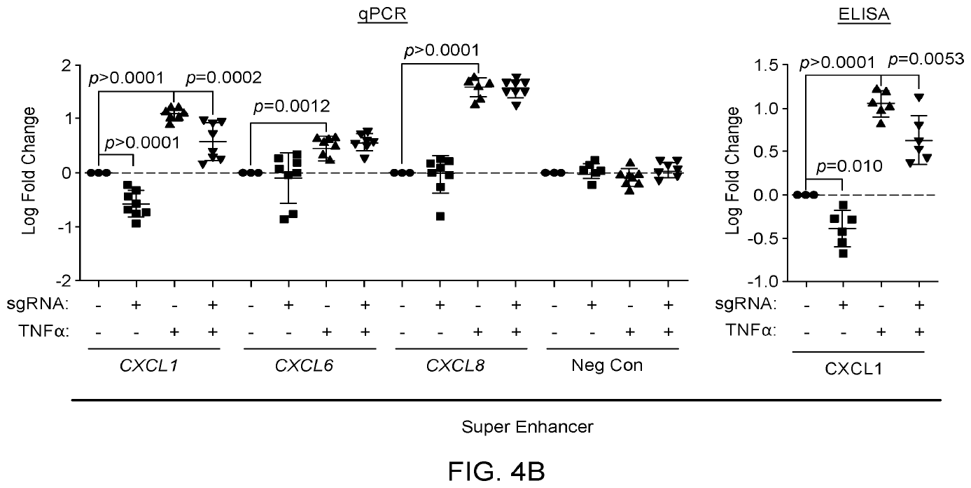

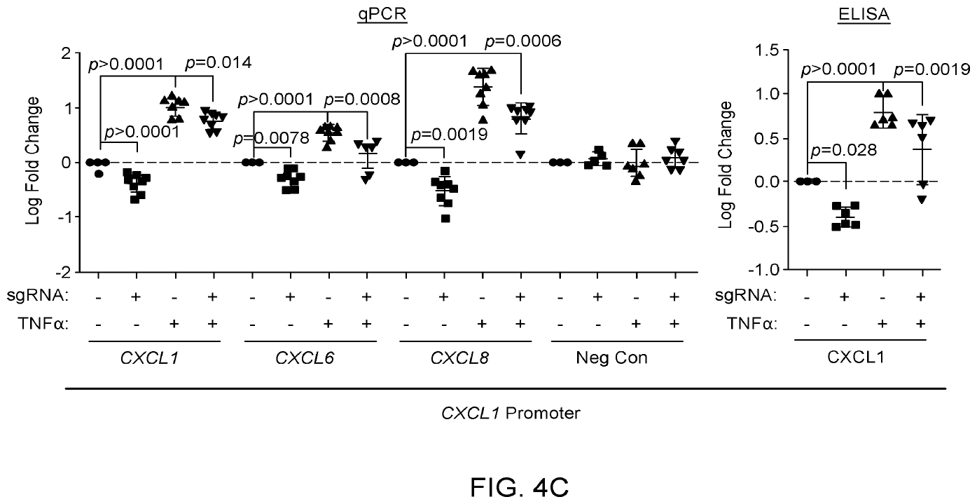

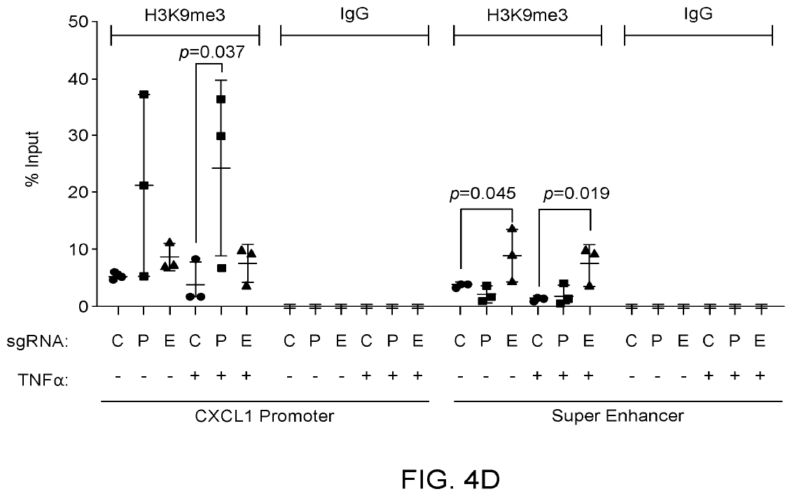

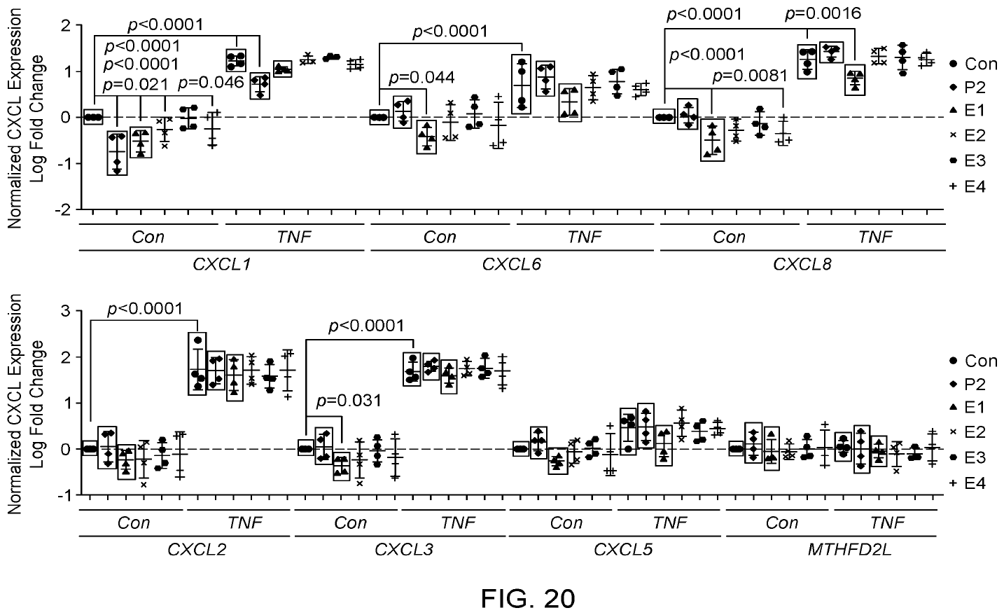

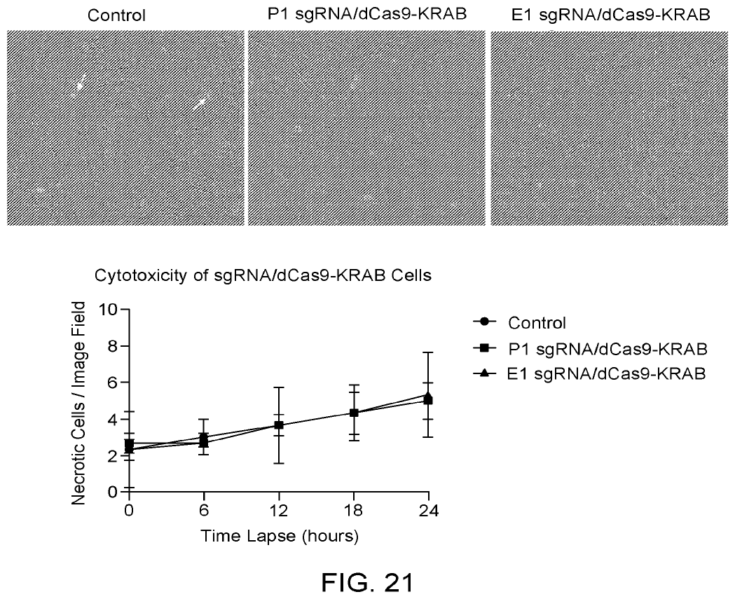

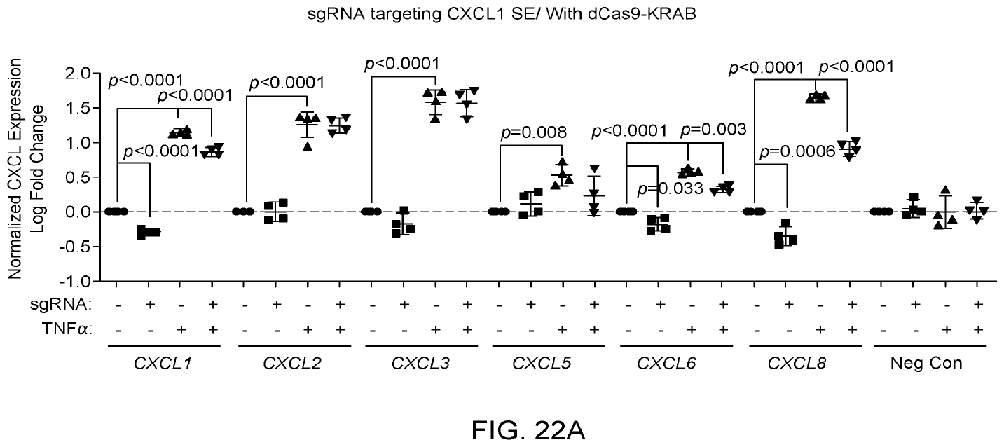

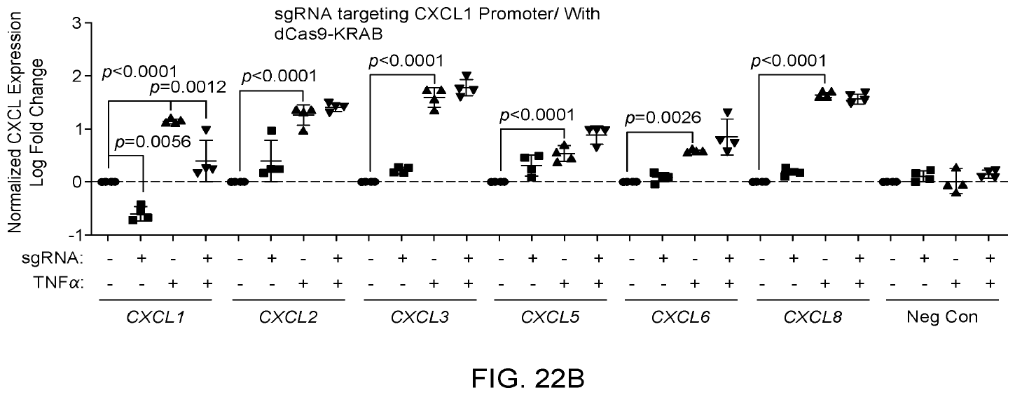

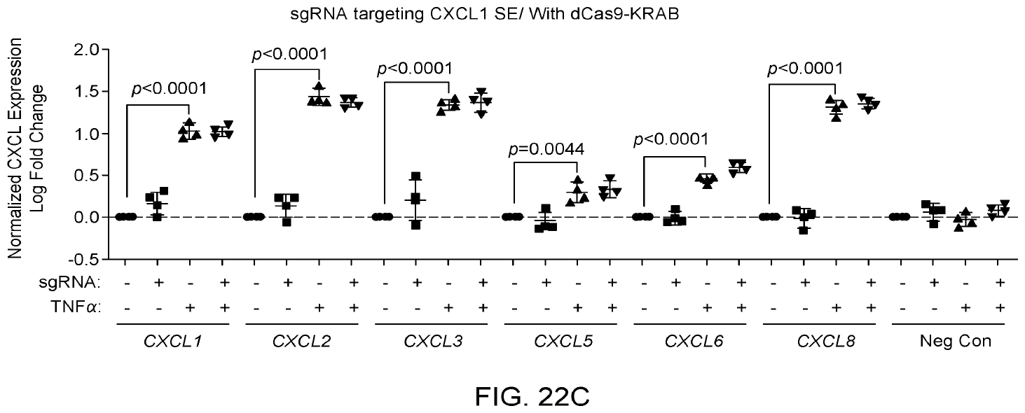

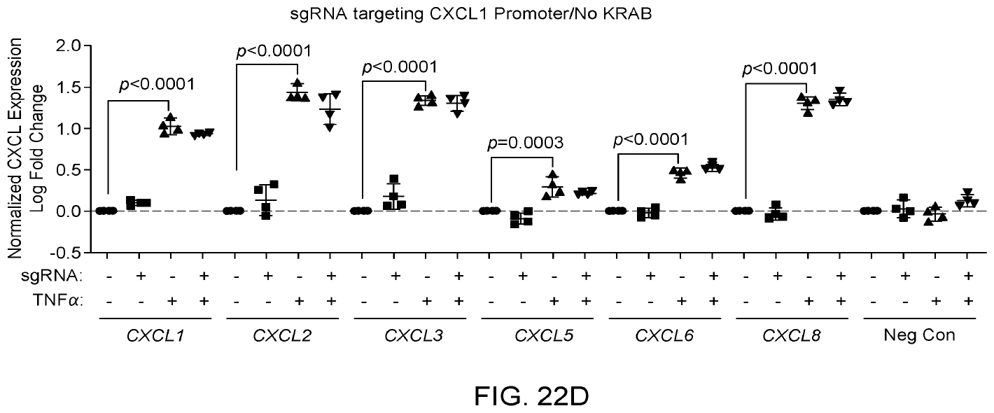

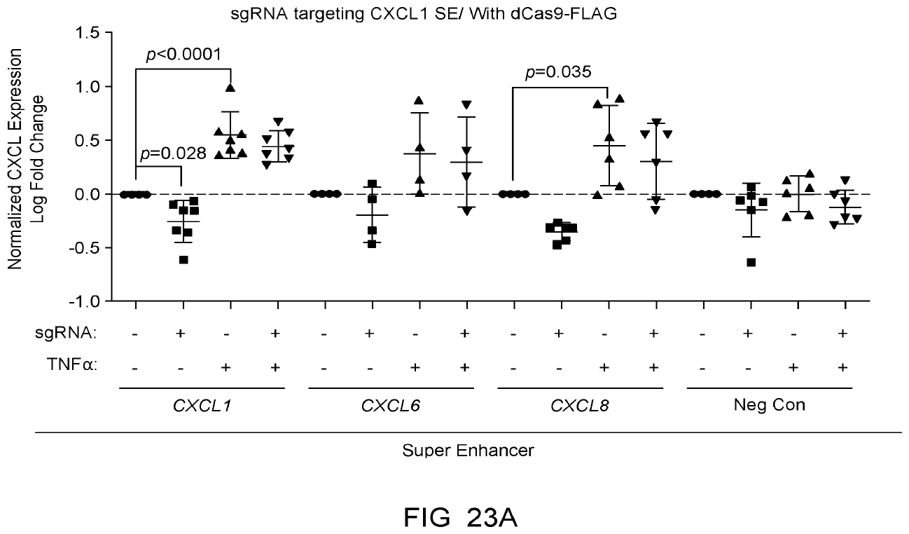

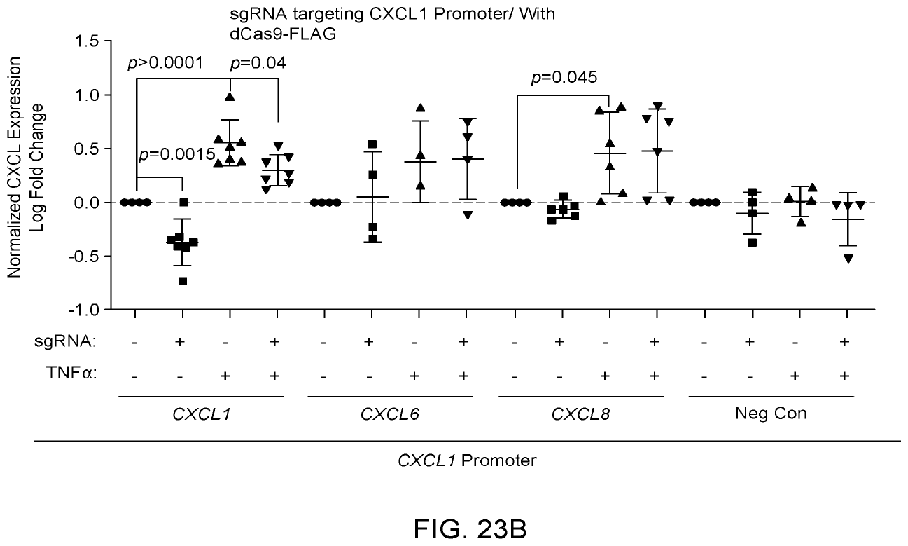

To demonstrate that targeted suppression of this super enhancer at strategic sites inhibits CXCL gene expression, an endonuclease-deficient Cas9 protein (dCas9) fused with the Krüppel associated box (KRAB) domain was used to introduce targeted, epigenetic gene suppression in the super enhancer region in LSECs (FIG. 4A). Single guide-RNAs (sgRNA), which dictate site-specificity of dCas9-KRAB, were designed to target the NF-κB binding sites on the super enhancer described above, and the sgRNA with the strongest effect compared to empty sgRNA vector in dCas9-KRAB transduced cells was selected for subsequent experiments (FIG. 20). It was found that dCas9-KRAB significantly reduced expression of multiple CXCLs without excessive cytotoxicity (FIG. 21), while the expression of nearby non-inflammatory gene MTHFD2L was unchanged (FIG. 4B). CXCL1, 6, and 8 appeared to be more sensitive to dCas9-KRAB mediated suppression of the CXCL super-enhancer compared to CXCL 2, 3, and 5 (FIG. 22A). Site-specific gene repression targeting a predicted NF-κB binding site in the promoter of CXCL1 was also performed. CXCL1 expression was suppressed, but other CXCL genes and a nearby non-inflammatory gene were unaffected (FIG. 4C, FIG. 22B). Cells receiving sgRNA treatment only without dCas9-KRAB showed no change in expression of CXCL genes (FIG. 22C and 22D). To demonstrate that the effect of dCas9-KRAB was target-specific, the enrichment of the repressive mark H3K9me3 was examined. H3K9me3 occupancy at the targeted NF-κB binding sites in the super enhancer and CXCL1 promoter regions was increased with dCas9-KRAB-mediated epigenomic editing (FIG. 4D). Interestingly, the expression of dCas9-FLAG construct without the KRAB domain showed a similar pattern of repression, but at to a lesser extent (FIG. 23). No significant difference in chromatin interaction was noted by 3C by dCas9-KRAB targeting (FIG. 19B). These experiments reflect the broad regulatory activity of CXCL super enhancer on multiple CXCL, genes, whereas repression of the CXCL1 promoter has more specific effects exclusively on the CXCL1 gene. The occupancy of H3K27ac through ChIP-seq and ChIP-qPCR in LSECs was also examined. H3K27ac occupancy in LSECs was increased with TNFα stimulation and decreased with celastrol, which decreases NF-κB and BRD4 binding, suggesting that H3K27 acetylation may be dependent on NF-κB and BRD4 binding (FIG. 17A and FIG. 18B).

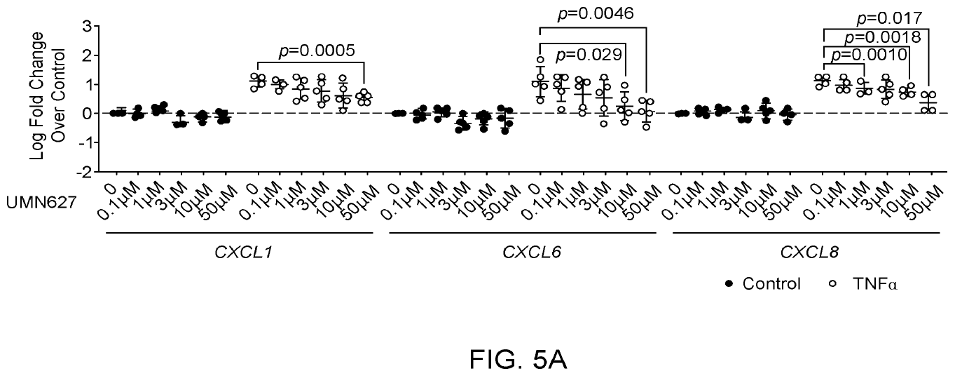

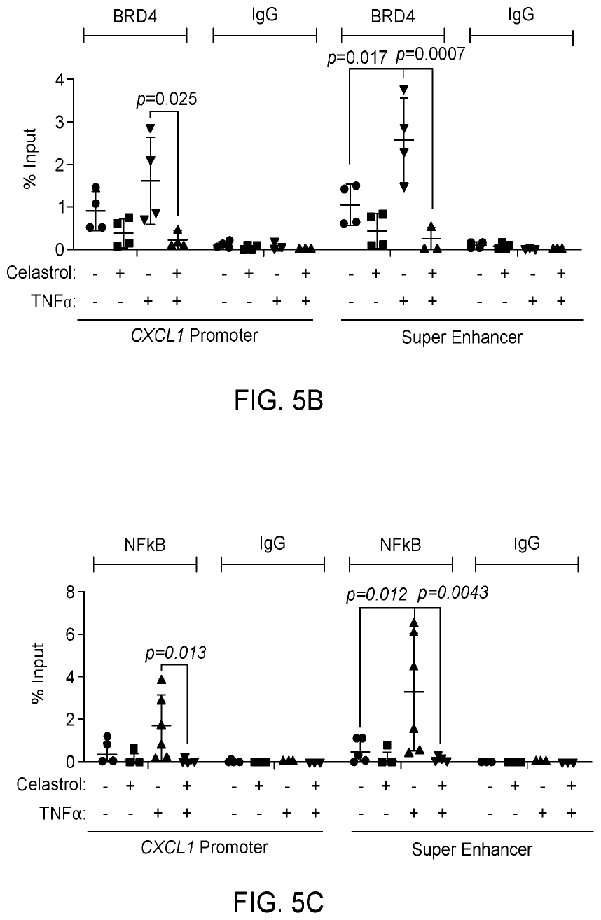

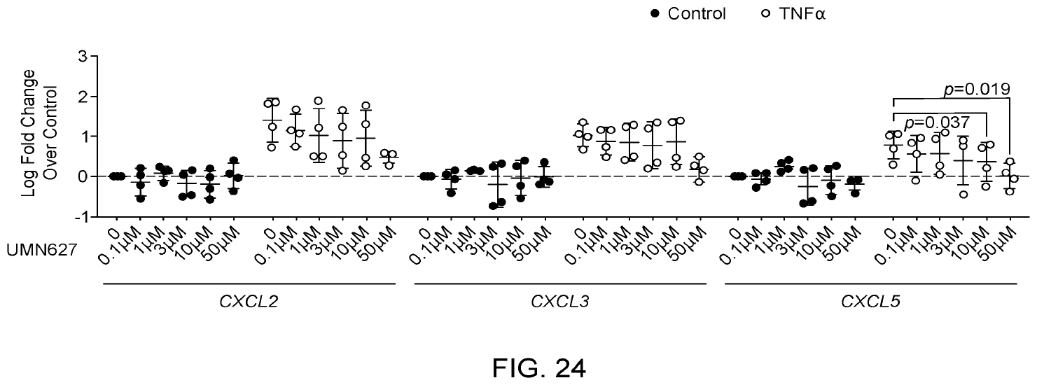

To further validate the function of this super enhancer in CXCL production, the impact of disrupting super enhancer signaling pathway function was examined using pharmacologic inhibitors. The transcription regulator and epigenetic reader BRD4contributes to super enhancer function by maintaining super enhancer structure and facilitating the recruitment of other transcriptional cofactors. A BD1 selective inhibitor, UMN627, which has over 20 fold higher affinity for BRD4 BD1 over BRD4 BD2 (IC₅₀=0.94 vs. 25.8 μM) (FIG. 24), was tested on LSECs for its ability to suppress the activity of the CXCL super enhancer. In vitro, UMN627 showed efficacy in CXCL suppression (FIG. 5A, FIG. 24).

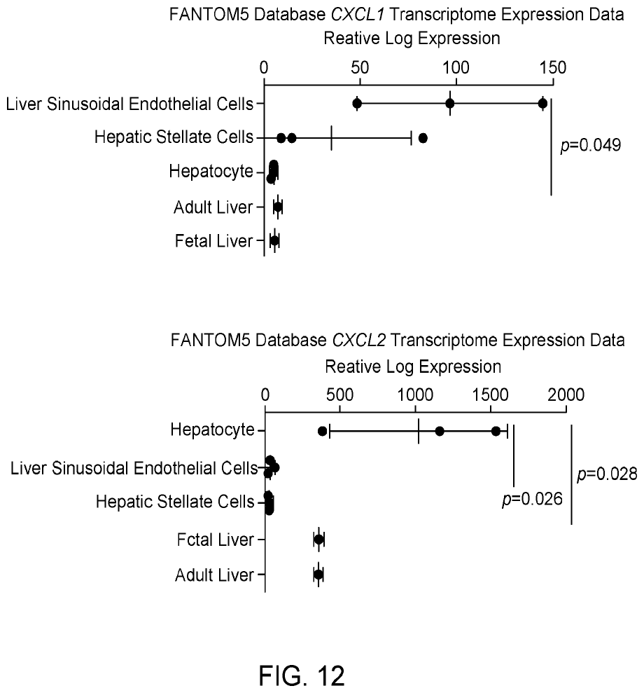

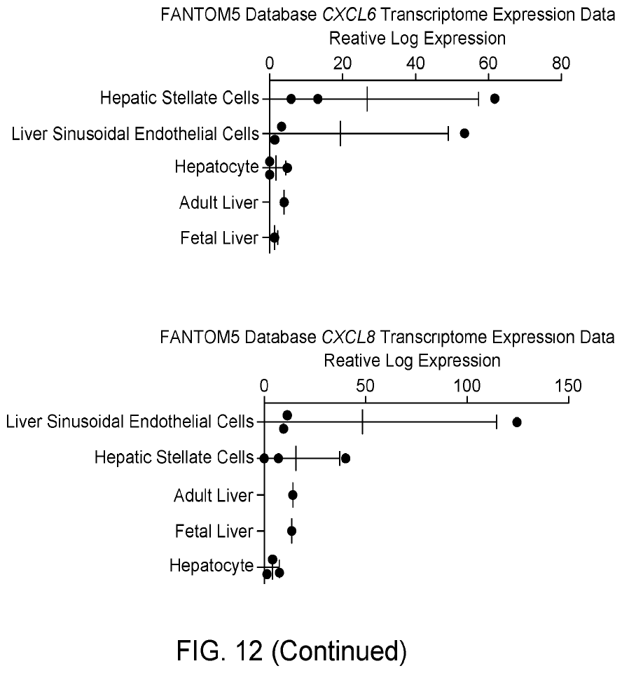

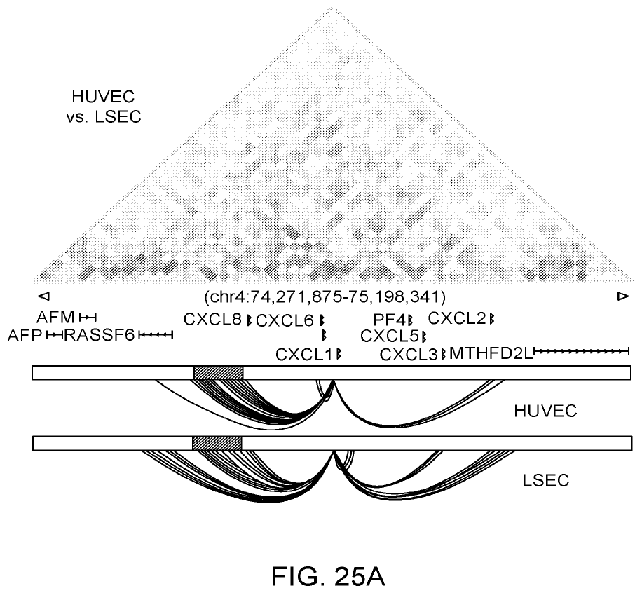

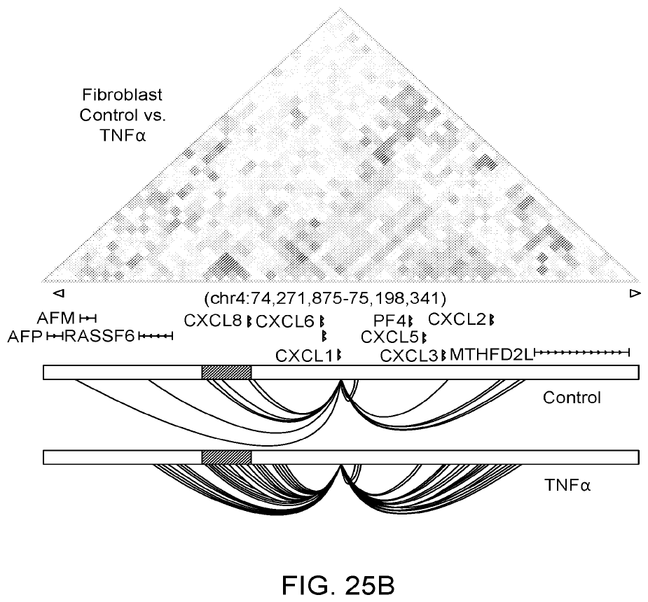

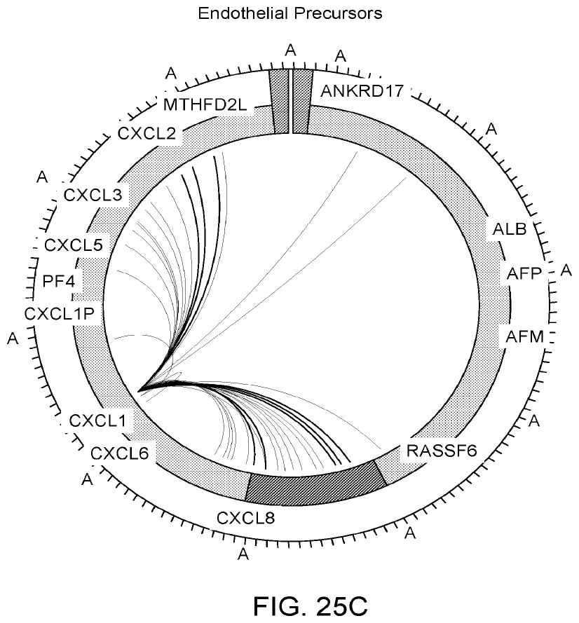

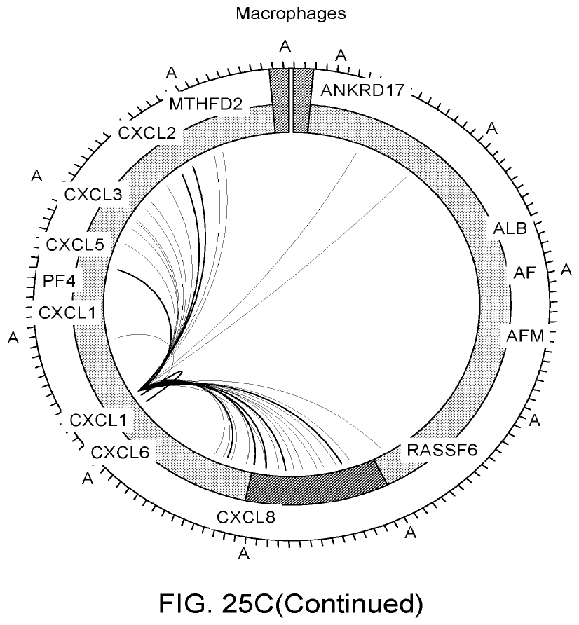

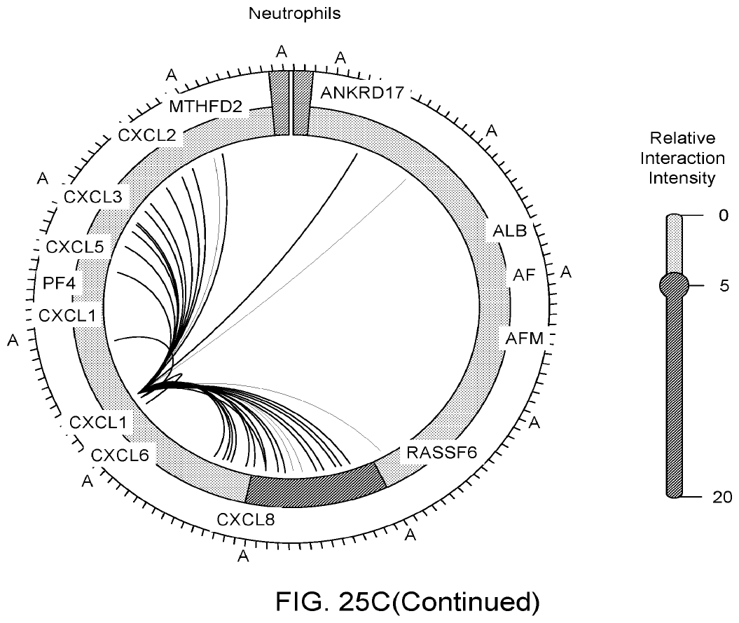

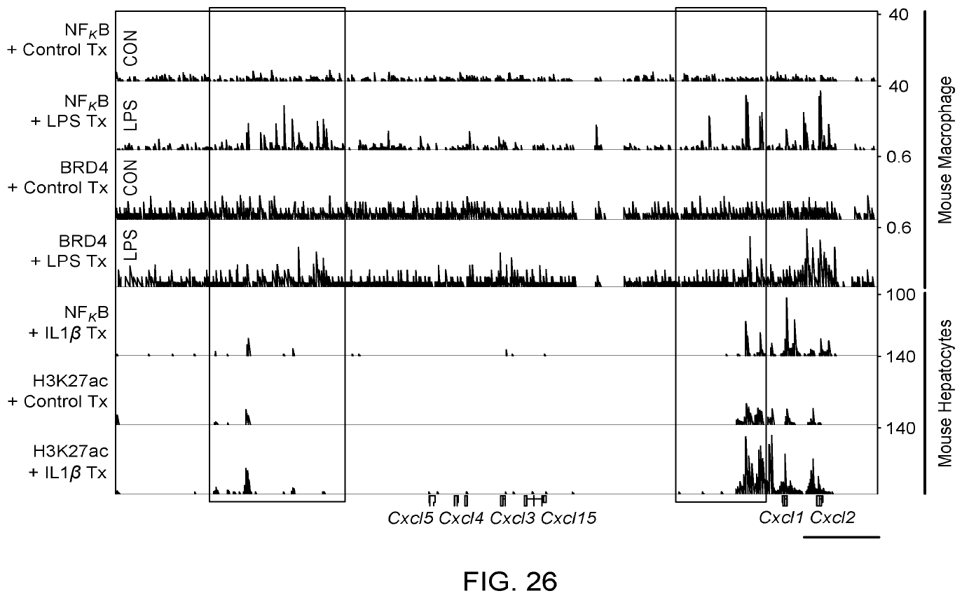

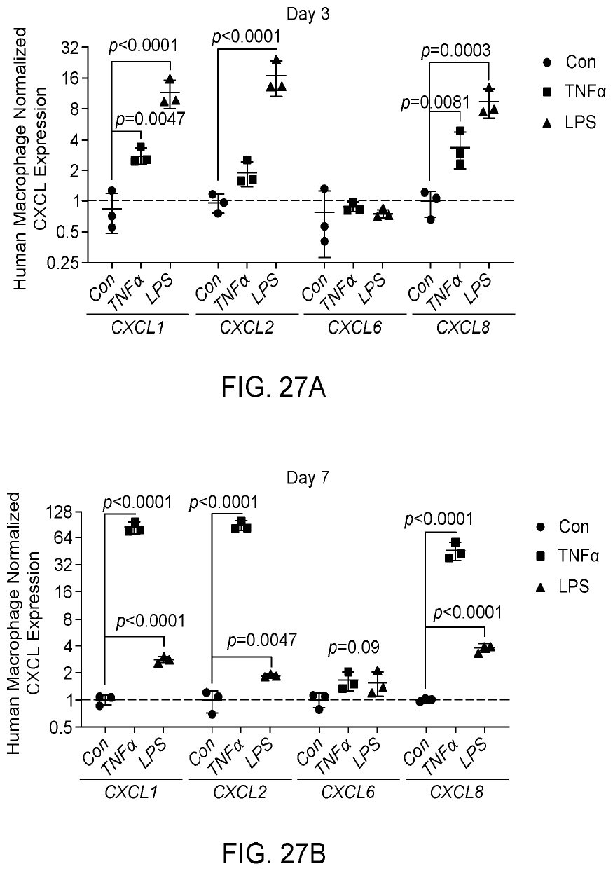

Considering the heterogenous sources of CXCL production in liver diseases (FIG. 12), cell type specificity of this super enhancer was examined by analyzing ChIP-Seq and Hi-C datasets from different cell types. The analysis of Hi-C data identified significant overlap of topologically-associated domains (TADs) between HUVEC and LSECs at the CXCL locus, suggesting a similar chromatin organization (FIG. 25A). In human lung fibroblasts, TNFα stimulation was found to enhance chromatin interactions between the CXCL super enhancer and CXCL genes (FIG. 25B), indicating the activation of the CXCL super enhancer. Another Hi-C study of human blood progenitor cells revealed similar enrichment of chromatin interactions in endothelial precursors, activated macrophages, and neutrophils (FIG. 25C). There was no enriched interaction in anti-inflammatory macrophages, erythrocyte precursors, and naïve B cells, among others. Furthermore, there appears to be a homolog for this super enhancer in mouse. ChIP-Seq data also showed the enrichment of NF-κB and BRD4 occupancy within the 2 putative super enhancer regions after LPS stimulation in bone marrow derived macrophages in mice (FIG. 26). Experiments with cultured macrophage support these observations as well. While isolated human circulating monocytes demonstrated poor response to TNFα stimulation, culturing monocytes with M-CSF induces differentiation into macrophage, and these cells become highly responsive to LPS and TNFα stimulation by upregulating chemokine expression (FIG. 27). In IL1β stimulated hepatocytes, ChIP-Seq identified enriched occupancy of NF-κB and H3K27ac at the same 2 putative super enhancer regions (FIG. 26). Taken together, these high-throughput epigenomic studies in both human and mouse support a conserved role for the CXCL super enhancer in regulating CXCL genes in immune cells and hepatocytes, as demonstrated in endothelial cells here.

### BET Inhibition Reduces CXCL Expression and Neutrophilic Infiltration in AH

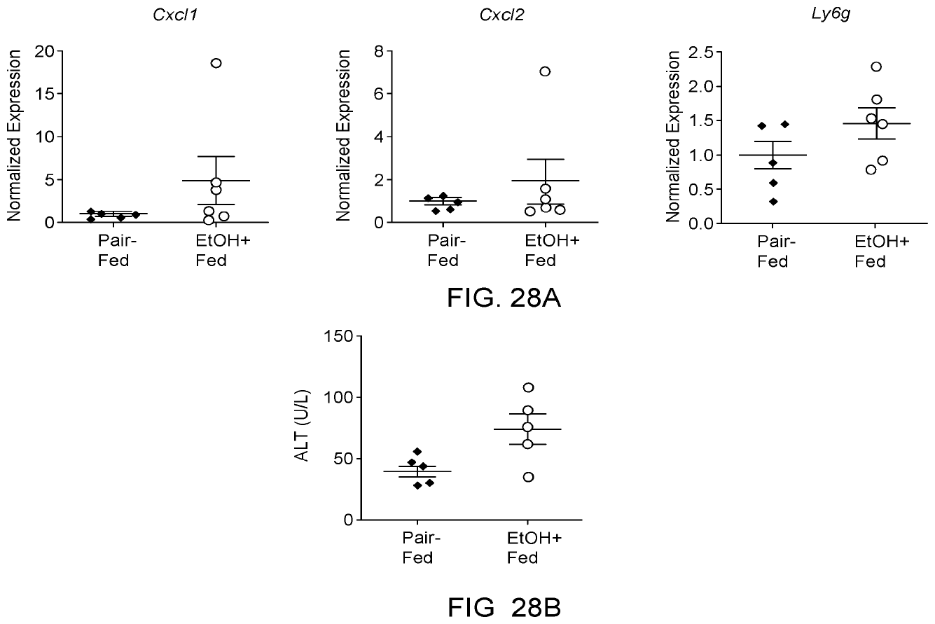

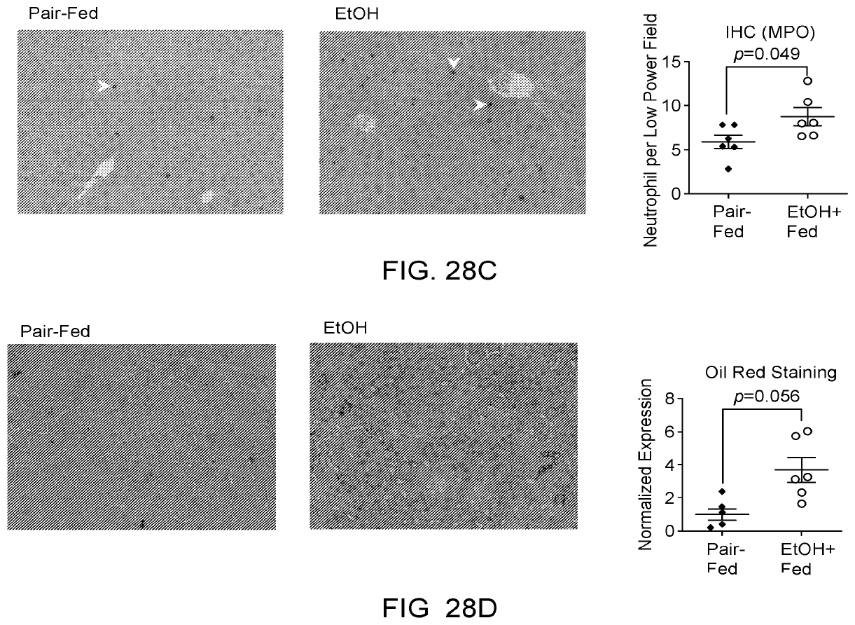

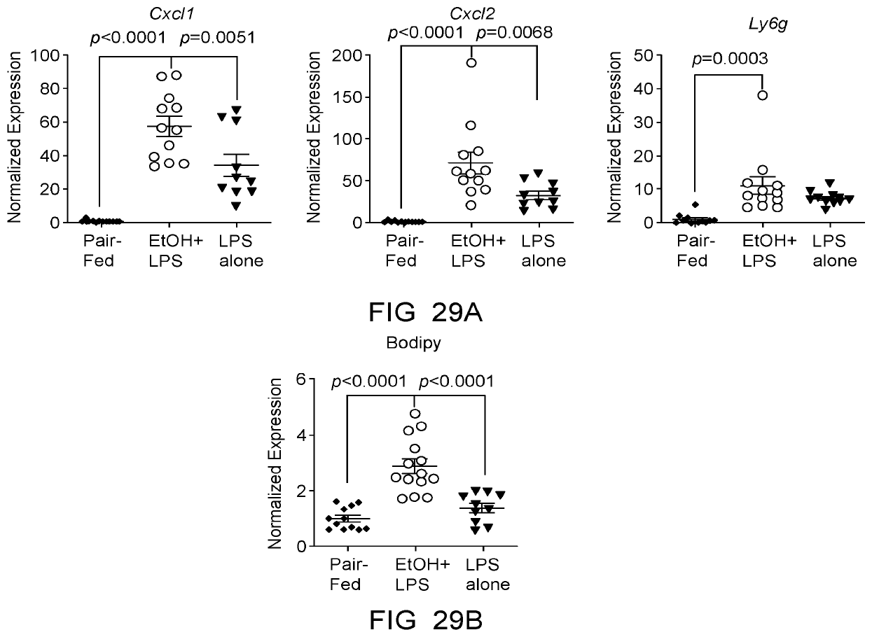

The effect of super enhancer suppression was examined in vivo. Using the NIH/NIAAA 10 days chronic-binge alcohol feeding protocol, the histopathology of human alcoholic hepatitis was mimicked in mice. In this model, increased murine Cxcl1 gene expression was observed, but Cxcl2 expression was not increased. IHC demonstrated modestly increased neutrophil infiltration with alcohol feeding (FIG. 28). Cxcl1 and Cxcl2 are functional homologs of human CXCL8 and play important roles in neutrophil chemotaxis in mouse. There was increased steatosis in alcohol-fed mice as assessed by Oil-Red-O staining (FIG. 28D).

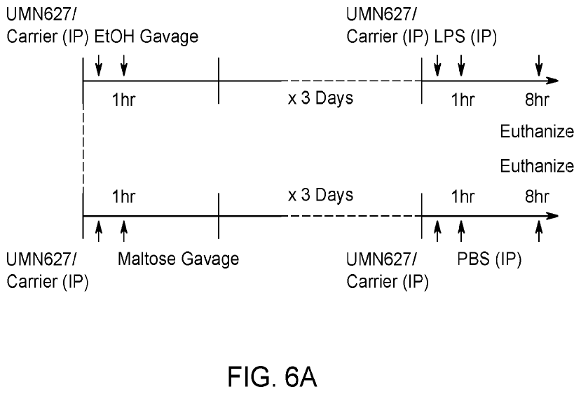

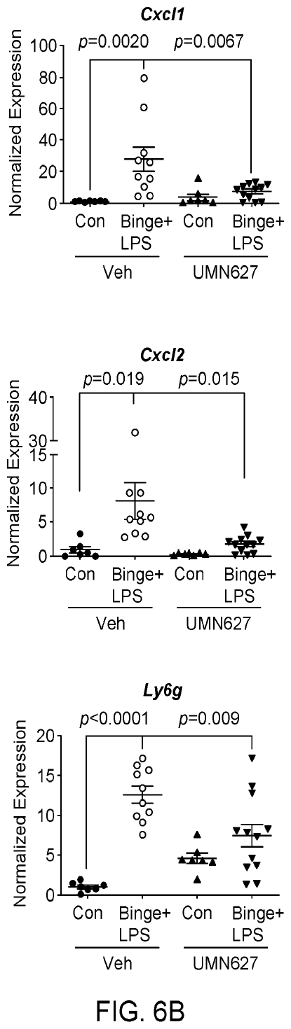

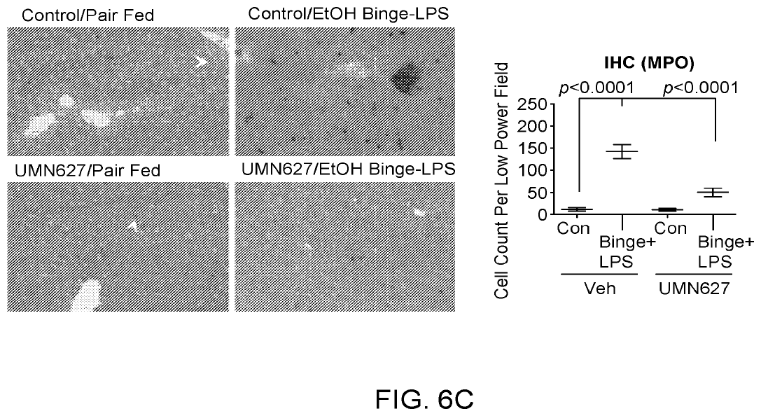

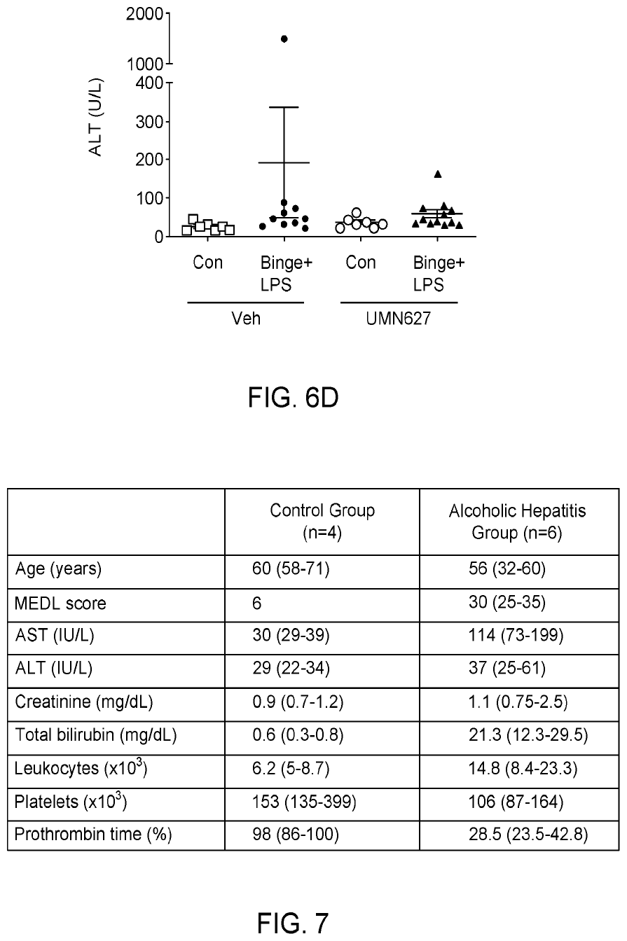

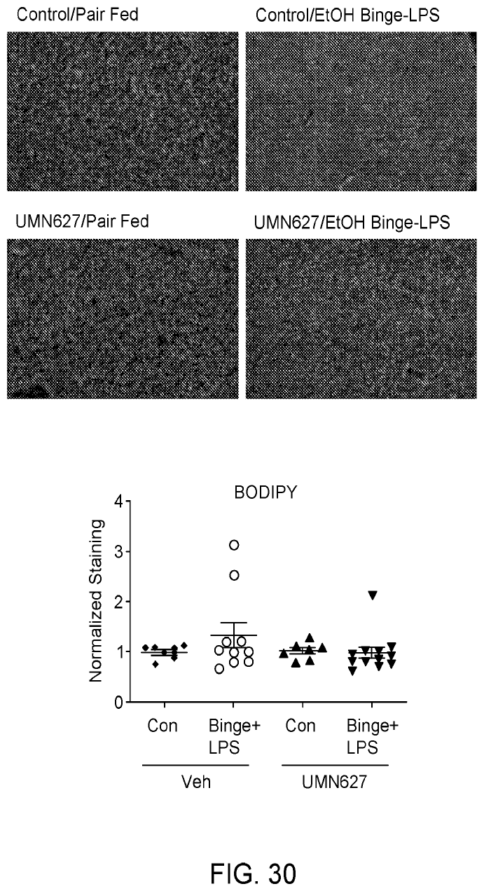

A multiple alcohol binges/LPS injection model was used to accentuate liver injury caused by LPS injection (FIG. 6A). Using this model, robust liver inflammation with increased CXCL expression and neutrophilic infiltration were induced in alcohol binges/LPS mice (FIGS. 6B and 6C). To ascertain if BD1-specific inhibition was sufficient in attenuating liver inflammation in this AH disease model, UMN627 was administered to mice undergoing alcohol binges/LPS injection. Attenuated CXCL expression and neutrophil infiltration were observed with the administration of UMN627 (FIGS. 6B and 6C). There were no significant changes in ALT levels or steatosis among various treatment groups (FIG. 6D and FIG. 30).

Together, these results demonstrate that inhibition of the BD1 domain of the BRD4 polypeptide can attenuate alcohol/LPS-induced liver inflammation, and can be used to treat AH.

### Materials And Methods

### Human Subjects

For human liver RNA-Seq and ChIP-Seq analyses, human liver explants from six patients with AH were procured at the time of liver transplant surgery. Four control patients with no history of chronic liver diseases undergoing liver resection for other causes were obtained to serve as controls. Select demographical information on these patients is shown in FIG. 7.

### RNA Seq and Histone Mark ChIP Seq

Human liver tissues were processed for RNA-Seq and Epigenomics Development Laboratory for ChIP-Seq on H3K27ac, H3K4me1, H3K4me3, and H3K27me3 marks. RNA-seq utilized samples from all six patients, and ChIP-seq was performed on five of six patients.

RNA-seq data were analyzed using the MAP-RSeq pipeline. In brief, paired-end reads were aligned to the human genome reference hg19 using TopHat (v2.1.0) and gene counts were estimated using the featureCounts (v1.4.6) software based on the Ensembl gene definition files. Gene expression was quantified as reads per kilobase per million mapped reads (RPKM). Protein-coding genes with RPKM>=1 in at least one sample were extracted and the top 5,000 genes with the largest between-sample variation were used in hierarchical clustering. A subset of protein-coding genes with reads per million (cpm)>=1 in at least two samples were selected for differential analysis. The differentially expressed genes between the 6 AH and 4 normal were identified using the edgeR package (v3.18.1) with the option of the trimmed mean of M-values (TMM) normalization, at the cutoffs of FDR<=1% and an absolute log2 (fold change)>=1.5.

ChIP was done in the Epigenomics Development Lab using antibodies against histone marks H3K27ac, H3K4me1, H3K4me3, and H3K27me3 with human liver tissue and against H3K27ac and NF-κB. Briefly, tissue (50 mg) is homogenized for 15-30 seconds in 500 μl of 1×PBS using tissue grinder. Homogenized tissues or tissue culture cells were cross-linked to final 1% formaldehyde for 10 minutes, followed by quenching with 125 mM glycine for 5 minutes at room temperature and by washing with TBS. The pellets were resuspended in cell lysis buffer (10 mM Tris-HCl, pH7.5, 10 mM NaCl, 0.5% NP-40) and incubated on ice for 10 minutes. The lysates were aliquoted into 2 tubes and washed with MNase digestion buffer (20 mM Tris-HCl, pH7.5, 15 mM NaCl, 60 mM KCl, 1 mM CaCl₂) once. After resuspending in 250 μl of the MNase digestion buffer with proteinase inhibitor cocktails for each tube, the lysates were incubated in the presence of 1,000 gel units of MNase (NEB, M0247S) per 4×10⁶ cells at 37° C. for 20 minutes with continuous mixing in thermal mixer (Fisher Scientific, 05-450-206). After adding the same volume of sonication buffer (100 mM Tris-HCl, pH8.1, 20 mM EDTA, 200 mM NaCl, 2% Triton X-100, 0.2% Sodium deoxycholate), the lysates were sonicated for 15 minutes (30 sec-on/30 sec-off) in Diagenode bioruptor and centrifuged at 15,000 rpm for 10 minutes. The cleared supernatant equivalent to the cellularity of 4×10⁶ cells was incubated with 2 μg of modification-specific antibodies on rocker overnight. The following antibodies are used in the experiment; anti-H3K27ac antibody (Cell signaling, 8173), in-house generated anti-H3K4me3 antibody (EDL lot 1), in-house generated anti-H3K4me1 antibody (EDL lot 1), and anti-H3K27me3 antibody (Cell signaling, 9733), anti-NF-κB (RELA subunit) (Cell signaling 8242S). For the next-generation sequencing, ChIP-seq libraries were prepared from 10 ng of ChIP and input DNAs with the Ovation Ultralow DR Multiplex system (NuGEN). The ChIP-seq libraries were sequenced to 51 base pairs from both ends using the Illumina HiSeq 2000 in the Mayo Clinic Medical Genomics Core.

ChIP-seq data was analyzed using the HiChIP pipeline. Briefly, paired-end reads were mapped to the hg19 genome reference using Burrows-Wheeler Alignment (BWA). Pairs of reads with one or both ends being uniquely mapped were retained and duplicates were removed using Picard MarkDuplicates command (broadinstitute.github.io/picard/). Narrow peaks from H3K4me3, H3K4me1 and H3K27ac were identified using model-based analysis of ChIP-seq (MACS2) software package at FDR<=1%. Broad peaks from H3K27me3 were identified using spatial clustering approach for the identification of ChIP-enriched regions (SICER) software package at FDR<=1%. For data visualization, Bedtools together with in-house scripts were used to generate per-million read density profile at 200-bp window size and a step size of 20 bp. ChIP-seq signal tracks were visualized in Integrative Genomics Viewer (IGV) software.

For each of the ChIP-seq libraries, the number of reads in the TSS±2kb region of all protein-coding genes was estimated, normalized to 10 million uniquely mapped reads (RP10M), log2 transformed and quantile normalized across samples. The normalized values extracted for the differentially expression genes were used to generate the heatmaps. In addition, the read density (RPM, reads per million) in 100-bp non-overlapping bins over the TSS±5kb region was calculated using the ngs.plot tool (v2.02). The input-subtracted read density was plot separately for the AH up- and down-regulated genes. Finally, for each of the histone marks, peaks present in at least two samples were retained and those showing increased or decreased signals in AH relative to normal were identified by the DiffBind package (v2.4.8) at the FDR<=1% and absolute fold change>=2. The retained peaks were assigned to the proximal (TSS±2kb) or to the nearest distal regulatory regions outside of TSS±2.5kb. Upregulated genes with increased signals of active marks (H3K4me3, H3K4me1 and H3K27ac) or decreased signal of H3K27me3, and downregulated genes with the reverse histone mark patterns were deemed to be genes of interest in the integrated analysis.

A heatmap was generated from the Z-scores of expression FPKM values with genes in the integrated analysis. Z-scores were calculated by subtracting the mean FPKM values in all subjects and dividing by the standard deviation. Genes in the integrated analysis were analyzed with Ingenuity pathway analysis (IPA) to uncover common regulatory pathways. Upregulated genes from the integrated analysis were analyzed separately. A canonical pathway unrelated to the liver (sperm motility) was removed, and top differentially activated pathways along with predicated upstream regulators were displayed. To explore the potential roles of TNFα and NF-κB signaling in AH differentially expressed genes, gene set enrichment analysis (GSEA) were performed using default settings (1000 permutations and for a maximum size of sets of 1000) on AH differentially upregulated genes as previously described. To examine the within-group variations of AH and normal subjects enlisted in the study, principle component analyses (PCA) were performed separately for the whole transcriptome, differential genes in the integrated analysis, and genes in the granulocyte and agranulocyte adhesion and diapedesis pathways. Expression level (RPKM values) of all cataloged genes were filtered and lowly expressed genes (<1 RPKM) were removed. Z scores were calculated from RPKM, and data was analyzed with built-in R function prcomp and plotted with package ggplot2 and rgl.

Super enhancer calling was performed using H3K27ac data of LSEC and HUVEC cells using the ROSE tool (v1.0.0, younglab.wi.mit.edu/super_enhancer_code.html), using H3K27ac peaks called with the Model-based analysis of ChIP-Seq (MACS) software package (v2.0.10). Peaks within TSS+/−2.5kb were excluded to account for promoter biases, and the remaining peaks within a distance of 12.5kb or less were stitched together.

### Human Liver Cell RNA-seq

Human primary liver cells used for RNA-seq were purchased from ScienCell. Human LSECs were isolated from mixed primary cultures containing all liver cells by CD31 antibody and characterized by immunofluorescence with antibodies specific to vWF/Factor VIII and CD31 (PECAM). LSECs are negative for HIV-1, HBV, HCV, mycoplasma, bacteria, yeast and fungi. RNA-seq was performed and analyzed in the same manner as described above for human liver RNA-seq. The RPKM values of CXCL chemokines were extracted and normalized to multiple house-keeping genes (Clorf43, CHMP2A, GPI, PSMB2, PSMB4, RAB7A, VCP, and VPS29).

### Analysis of Public Hi-C Data of Human Cells

Hi-C data in HUVEC (GSM1551629), LSEC (ENCLB284TIY) and fibroblasts (GSM1055800 and GSM1055802) were accessed through the 3DIV Hi-C database. Heatmap shows the differences in normalized interaction frequency between the indicated cell types at the CXCL1 locus (FIGS. 18A and 18B). Promoter Capture Hi-C (CHi-C) data of human endothelial precursors, macrophages, and neutrophils were accessed from the CHiCP web browser.

### Analysis of Public scRNA-seq Data

scRNA-seq data of mouse and human liver were analyzed. Data was downloaded from GEO database and analyzed in Seurat V4. In studies by MacParland et.al. (GSE115469) and Aizarani et.al. (GSE124395), clustering information of single cells were provided by the authors. Data matrixes were analyzed in Seurat, and cell clustering was performed with marker genes provided by each respective study. For each study, the total count of CXCL chemokines transcript was tallied per cell type and relative contribution of each cell type was calculated as a percentage.

### Cell Culture and TNFα Stimulation

Primary human LSECs were purchased from ScienCell (Cat #5000) and cultured using standard cell culture techniques. For liver cell RNA-Seq experiment, primary human LSECs, HSCs (Cat #5300), and HiBECs (Cat #5100) were purchased from ScienCell, and HepG2 cell line was obtained from ATCC (HB-8065). Where appropriate, LSECs underwent TNFα (Peprotech, 300-01A) stimulation at 20 ng/mL. In selected experiments, cell supernatant was collected and enzyme-linked immunosorbent assay (ELISA) performed to assess concentration of secreted CXCL1.

### Additional Cell Culture Techniques

LSECs were thawed and cultured according to standard cell culture conditions and according to manufacture instruction (ScienCell). Briefly, LSECs are plated at low confluency and cultured in Endothelial cell medium (Cell 211-500). Fresh medium changed every other day and cells are split when close to confluency. For TNFα stimulation experiments, low passage cells were plated at 70% confluency and cultured overnight. Serum starvation was performed by changing cells to low-serum medium (0.5% FBS in basal endothelial medium (Lonza CC-3121)) for 2 hours. Human TNFα at concentration 10 ng/mL was added to low-serum medium and incubated with cells for 90 minutes before cells were collected and assayed for downstream analysis. For experiments with celastrol (Sigma C0869) or BRD4 inhibitor UMN627, LSECs were plated and serum starved similarly to TNFα stimulation experiments. Inhibitors were added at concentrations indicated to low-serum medium, and cells were incubated in inhibitor containing medium for 2 hours. After inhibitor treatment, medium change was performed with TNFα (Peprotech 300-01A) at 10 ng/ml or control low-serum medium for 90 minutes incubation. LSECs were then collected for analysis.

Primary human monocytes (Astarte 1008) were thawed and resuspended in RPMI (Gibco 1640) with 10% bovine FBS supplemented with recombinant human M-CSF (BioLegend 574804) at 20 ng/mL. Medium was changed on Day 1 then every 2 days and cells were cultured for up to 7 days.

### CRISPR-dCas9 Lentiviral Transduction

Multiple sgRNA target sites were selected in the promoter area of CXCL1 gene for dCas9-KRAB targeting assay. Putative target sites were selected based on predicted NF-κB binding motif analysis with JASPAR. The top five sites predicated to show the highest NF-κB binding affinity were chosen, and sgRNA sequence was designed using the publicly available Benchling software in proximity to these target sites. Synthesized sequences were inserted into the sgRNA backbone vector LentiGuide-Puro (Addgene Plasmid #52963). The backbone vector was used as a control. The dCas9-KRAB Lentivector (Addgene Plasmid #89567) or a dCas9-FLAG Lentivector (Addgene Plasmid #106357) were also obtained through Addgene. 293T cells were cultured and transfected with either modified a sgRNA lentivector or a dCas9 lentivector according to manufacture protocol (Lipofectamine 3000, Invitrogen). Cells were cultured for 48 hours and supernatants containing lentivirus were collected. dCas9 lentivirus containing supernatant was further concentrated 100-fold with ultracentrifugation at 120,000 g for 90 minutes (Optima XPN-80 Ultracentrifuge, Beckman Coulter). LSEC cells at low passage were transduced with supernatant containing sgRNA lentivirus along with 1:1000 dilution of polybrene (Millipore TR1003-G). Cells were cultured for 48 hours before selection with puromycin (puromycin resistance conferred by LentiGuide-Puro lentivector) (Sigma P8833). Selected cells were replated and transduced with either dCas9-KRAB or dCas9-FLAG lentivirus concentrate, and antibiotics selection was performed with either blasticidin (dCas9-KRAB) (Invitrogen ant-bl-1) or puromycin (dCas9-FLAG) on LSEC cells after 48 hours in culture. Selected cells were replated and treated with human TNFα and assayed by qPCR for CXCL gene expression.

### CXCL1 ELISA

Human LSEC CXCL1 ELISA was performed on supernatant of cultured cells. Cultured cells underwent medium change with equal volume of culture medium for 16 hours before cells and supernatant media were collected. Capture ELISA was performed on supernatants using the Human CXCL1 DuoSet ELISA kit (R&D, DY275) using manufacturer's instructions. Fresh medium was used as negative control. Cells were collected and lysed in RIPA buffer (Cell Signaling 98065) and protein concentration was quantified with DC Protein Assay (BioRad 500-0114) according to manufacture protocols. CXCL1 concentrations determined by ELISA assays in supernatant was then normalized to protein concentration to ensure equal plating.

### Real-Time PCR

mRNA levels were quantified by real-time reverse transcription PCR. RNA extraction was performed with RNeasy kit (Qiagen 74104) from cells and mouse tissue according to the manufacturer's instructions. RNA Quantification was performed with spectrophotometry (NanoDrop, Thermo Scientific). 500 ng of mRNA was used for cDNA synthesis with dNTP and oligo primer using SuperScript™ III (Invitrogen 18080-093) for reverse transcription per the manufacturer's protocol. Real-Time PCR was performed from cDNA using IQ SYBR Green Mix (Biorad 1725121) on the 7500 Real-Time PCR system (Applied Biosystems), according to the manufacturer's instructions. Amplification of GAPDH and B-actin was performed for respective samples as internal controls. Each experiment was done in duplicates.

### Neutrophil Chemotaxis Assays

Neutrophil Isolation: Human neutrophils were isolated from whole blood using an immunomagnetic separation technique (Miltenyl Biotec 130-104-434 and MACSxpress Separator) according to manufacture protocol. Following isolation, cells were suspended in RPMI (Gibco 1640).

Microfluidic Device fabrication: Microfluidic devices were fabricated using standard soft lithography approaches. Design of the device with 6 parallel channels was created in AutoCAD and converted into photomask by CAD/Art services (Bandon, Oregon) (FIG. 14A). Microfluidic channels were then molded in polydimethyl siloxane (PDMS) and secured atop a 3×1 inch glass slide. A cloning cylinder was mounted at the inlet of each of the six channels and was used for loading neutrophils. Prior to seeding cells, microfluidic chambers were infused with 10% FBS for 30 minutes. Subsequently, FBS was removed and devices were washed twice with ice cold PBS and coated with collagen. LSECs were seeded into devices and cultured for 2 days prior to use.

Chamber Chemotaxis Experiment: Neutrophils were stained with Hoechst dye (Invitrogen 33342) for visualization and then infused into a microfluidic device containing LSECs for 10 minutes at the flow rate of 3.84 L/minute (shear stress of 0.02 Pa) for 10 minutes. In select samples, neutrophils were resuspended in medium supplemented with recombinant human CXCL1 (STEMCELL 78063.1) at 100 ng/ml or in conditional LSEC medium (medium exposed to LSECs for 24 hours). Afterwards, microfluidic devices were washed by flowing PBS for additional 10 minutes. The neutrophils attached in microfluidic channels were quantified by acquiring images at 10× magnification, 5 images per channel.

Transwell Chemotaxis Experiment: LSECs were seeded in Transwell plates (Corning 3421) and cultured for 24 hours with 600 μL of basal, TNFα (10 ng/mL) supplemented medium, or TNFα and celastrol supplemented medium. Prior to the experiment, overnight medium was removed from the negative (basal medium) and positive (recombinant CXCL1) controls and appropriate fresh medium was added. 600 μL of endothelial medium was added back to each control well, and recombinant CXCL1 at 100 ng/mL was added to positive control wells. Isolated neutrophils (1 million cells) were added to each well insert. Chemotaxis experiment was allowed to occur at 37° C. for 1 hour. Inserts were removed and plates were imaged in IncuCyte at 10× magnification with 5 visual fields. Quantification of neutrophils were done manually.

### Circular Chromosome Conformation Capture-Sequencing (40-Seq)

10 million LSEC cells with or without TNFα treatment or HEK293T (ATCC CRL-11268) cells were fixed in 1×PBS/10% FCS, 2% (w/v) final concentration formaldehyde for 10 minutes at room temperature. A final 0.125 M Glycine was added to stop the fixation on ice, followed by 400 g×8 minutes in 40° C. to remove supernatant. Pellets were resuspended in cold lysis buffer (50 mM Tris-HCl pH 7.5, 150 mM NaCl, 5 mM EDTA, 0.5% NP-40, 1% TX-100 and 1× complete protease inhibitors (Roche #11245200)) and incubated 10 minutes on ice. The efficiency of cell lysis was determined by Methyl Green-Pyronin staining (Sigma #HT70116). The DNA was digested with Csp6I (New England BioLabs R0639) and NlaIII (New England BioLabs R0125) as primary and secondary enzymes, respectively. T4 DNA ligase (New England BioLabs M0202) was used for both ligation steps. Specific primers were designed at the CXCL1 gene promoter with 4C-CXCL1 reverse primer (Csp6I) and 4C-CXCL1 forward primer (NlaIII). PCR amplifications were made with Expand Long Template PCR System (Roche). The bar-coded DNA libraries were generated with Illumina primers for each sample and purified with a High Pure PCR Product Purification Kit (Roche) and sent for deep sequencing. The libraries were sequenced on an llumina HiSeq 2000 instrument (Illumina, CA, USA). 4C libraries were sequenced to 100 bp from both ends. Primer sequences were trimmed off using the Trim Galore package (v0.2.2, bioinformatics.babraham.ac.uk/projects/trim_galore/). Only the pairs of reads whose primer sequences were trimmed were retained. The retained reads were mapped to the human genome reference hg19 using BWA-MEM (v0.7.10) 9 (arxiv.org/abs/1303.3997) in single-end mode. The mapped reads were filtered to keep those with a minimal mapping quality score of 20. Cis interactions within 1 Mb of the reference region were identified using the R package Basic4Cseq. Illumina adaptors were included in the primer sequences.

### Chromosome Conformation Capture (30)

10 million LSEC cells or CRISPR dCas9-KRAB cells (generated with methods provided below) with or without TNFα treatment was trypsinized and resuspended in 10% FBS/DMEM in single-cell suspensions. Cells were crosslinked with in 1×PBS/10% FCS, 2% (w/v) final concentration formaldehyde for 10 minutes at room temperature followed by addition of 0.125 M Glycine to stop the fixation on ice. Cells were pelleted and resuspended in cold lysis buffer (50 mM Tris-HCl pH 7.5, 150 mM NaCl, 5 mM EDTA, 0.5% NP-40, 1% TX-100 and 1× complete protease inhibitors (Roche #11245200)) and incubated for 10 minutes on ice. The efficiency of cell lysis was determined by Methyl Green-Pyronin staining (Sigma #HT70116). DNA was digested with NlaIII restriction enzyme (New England BioLabs R0125) overnight, and ligated with T4 DNA ligase (New England BioLabs M0202). DNA was then de-crosslinked overnight and purified with phenol-chloroform. A NlaIII restriction site near the predicted NF-κB binding site in the CXCL super-enhancer was used to design the reference sequence. Specific primers were designed to detect DNA fragment ligation at various restriction sites near each CXCL promoter NF-κB binding site and control segments RASSF6 and intronic segments between CXCL genes. The interaction frequencies of various segments were assessed by probe-based qPCR (PrimeTime, IDT), and amplification levels were normalized to RASSF6 and plotted (FIG. 3F and FIG. 19).

### ChIP

LSECs were treated with appropriate conditions as outlined separately, and subjected to ChIP according to Millipore High-Sens ChIP kit (Millipore MAGNA0025) manufacture protocols. Briefly, cells were crosslinked with formaldehyde (1% final concentration) followed by glycine treatment (100 mM) for 5 minutes each. Cells were washed, collected, pelleted with centrifugation, and lysed with cell lysis buffer. Cells were repelleted, the nuclei were lysed with provided nuclear lysis buffer, and DNA was sheared with ultrasonification. Soluble chromatin was aliquoted and immunoprecipitated with magnetic beads with anti-bodies for BRD4 (Abcam ab128874), NF-κB (Cell signaling 8242S), H3K9me3 (Abcam ab8898), or H3K27ac (Abcam 4729) with appropriate isotype controls. Immunoprecipitated beads were collected and processed according to manufacture protocol. Real-time PCR was performed in purified ChIP and input DNAs at target loci, and enrichment was compared with isotype control IgG.

### Immunohistochemistry

Right lobes of all mouse livers were fixed in 10% formalin and embedded in paraffin and cut into 5 μm sections. Slides were deparaffinized and underwent antigen retrieval (IHC-Tek IW-1000). Slides were treated with hydrogen peroxide for 10 minutes before blocking in 5% bovine serum albumin in phosphate-buffered saline (PBS) for 1 hour at room temperature. Samples were then incubated overnight at 4° C. with rabbit anti-MPO antibody (Abcam ab9535) at 1:25. Slides were then blocked with Avidin and Biotin (Vector SP-2001) for 15 minutes each before treatment with biotinylated secondary antibody horse anti-Rabbit (Vector BA-1100) at 1:200 for 1 hour at room temperature. Slides were then treated with

ABC reagent (Vector PK-7100) for 30 minutes before treatment with DAB reagent (Vector SK-4100) for 5 minutes. Counterstain was performed with hematoxylin stain and dehydrated in ethanol and xylene. Images were acquired using a Zeiss LSM 780 confocal microscope at 10× magnification, 5 images per slide (Carl Zeiss MicroImaging, Jena, Germany).

### Oil Red Staining and BODIPY Staining

Liver sections from the left lobe of all mouse livers were embedded in OCT (Sakura 4583) and flash frozen. Frozen tissue was sectioned into 10 μm slices, fixed in 10% formalin and stained with BODIPY 493/503 (Invitrogen D3922) or Oil-red (Sigma 00625) and counterstained with 4′,6-diamidino-2-phenylindole (DAPI) (Sigma D9542) or hematoxylin, respectively. Representative images were obtained with microscopy in one session under same settings (Carl Zeiss MicroImaging, Jena, Germany), 3 images per slide. The proportion of tissue stained with BODIPY, or Oil-red content was quantified with ImageJ macro commands for standardization. For BODIPY staining, FITC channel intensity was measured across the whole image, and average intensities of images were normalized to the average of all control samples. For Oil Red staining, red staining was quantified above a pre-selected threshold, and average intensities of images were normalized to the average of all control samples.

### Western Blot

Liver tissue was homogenized in RIPA lysis buffer (Cell signaling 9806S) with protease inhibitor cocktail (Roche 4693159001). 40 μg of protein were loaded onto a SDS-PAGE gel for electrophoresis and proteins were transferred onto a nitrocellulose membrane. The membrane was blocked by 3% bovine serum albumin and then incubated overnight with a primary antibody. Primary antibodies used include: anti-caspase 3 (Cell signaling 14220S) and anti-HSC70 (Santa Cruz, sc7298). Blots were developed using a chemiluminescence substrate (Santa Cruz sc-2048). HSC70 was used as the loading control and the results were quantified by using the ImageJ software.

### Lipid Peroxidation Assay and 8-Hydroxy 2 Deoxyguanosine (8-OHdG) ELISA

MDA quantification with Lipid Peroxidation Assay Kit (Abcam ab118970) was performed according to manufacture protocol. For 8-OHdG assay, mice liver DNA was purified with DNeasy Blood&Tissue Kit (Qiagen 69504) and treated with Nuclease P1 according to manufacture protocol. 8-OHdG ELISA (Abcam ab201734) was performed according to manufacture protocol.

### Animal Experiments

Multiple Alcohol Binge with LPS Model with BRD4 Inhibitor UMN627. WT C57BL/6 mice (10-12 weeks) were used in this model. All mice were fed standard chow and were gavaged once a day for 3 days with 6 g/kg alcohol solution or equal caloric maltose dextran solution. On Day 4, mice receiving alcohol gavages were given IP injection of LPS at 4 mg/kg, control mice were injected with equal volume of PBS, and all mice were sacrificed 8 hours later as described elsewhere (see, e.g., Beier et al., Hepatology, 49 (5): 1545-53 (2009)). UMN627 (22 mg/kg) in 10% KLEPTOSE® and 1% DMSO or equal volume of carrier solution were administered by IP injection to mice 1 hour before gavage each day or LPS/PBS injection on Day 4.

### Statistical Analysis

Means are expressed as means±standard deviation. Statistical analysis was conducted using GraphPad PRISM (La Jolla, USA) and R statistical software. Comparisons between three groups or more were conducted using one-way ANOVA with Dunnet's or Tukey's post-test for multiple comparisons using GraphPad PRISM. Comparisons with two different conditions were performed with two-way ANOVA with Sidak's or Tukey's post-test for multiple comparisons. A comparison of two groups was performed using the

Student's t test. P value≤0.05 is considered significant.

### Data Availability

All RNA-Seq and ChIP-Seq data generated in this publication will be uploaded online on the GEO database (GSE155926 and 166564).

### Example 2: BRD4 inhibitors and Liver Disease

### Results

### BRD4 Inhibitors Reduce Inflammatory Chemokines in Human Hepatic Sinusoidal Endothelial Cells

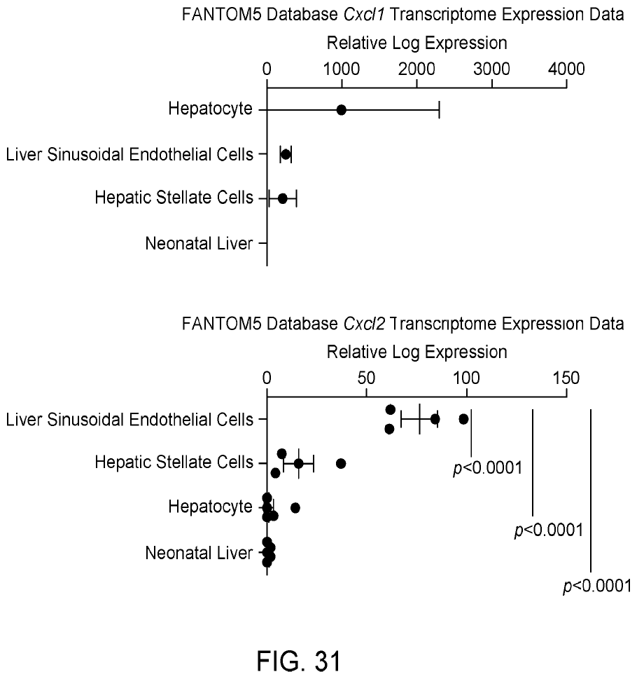

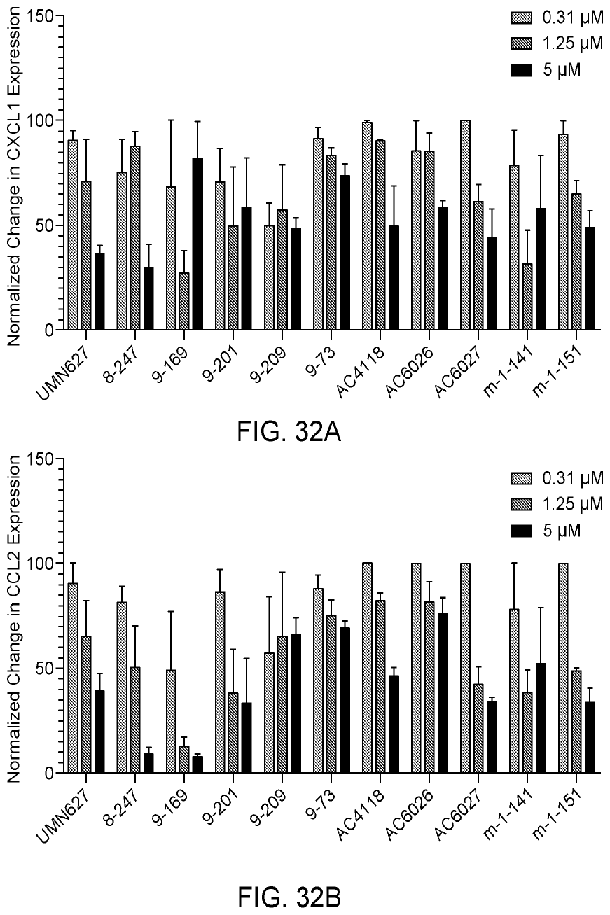

The efficacy of the BRD4 inhibitors was measured in an in vitro model of mild liver inflammation. The BRD4 inhibitors were incubated with human hepatic sinusoidal endothelial cells (HHSEC) prior to incubation with lipopolysaccharide (LPS). Quantitative PCR (qPCR) was used to measure the expression of the chemokines CXCL1 (neutrophils) and CCL2 (monocytes, memory T-cells, and dendritic cells) which recruit immune cells to site of inflammation (FIGS. 32A and 32B). At 0.31 μM most of the compounds (except 9-169) had little to no effect on the expression of CXCL1 and CCL2. The compounds AC6027, UMN627, AC4118, m-1-151, 9-201, and 8-247 showed dose dependent reduction in both chemokines, effectively decreasing chemokine expression below 50% compared to the LPS control.

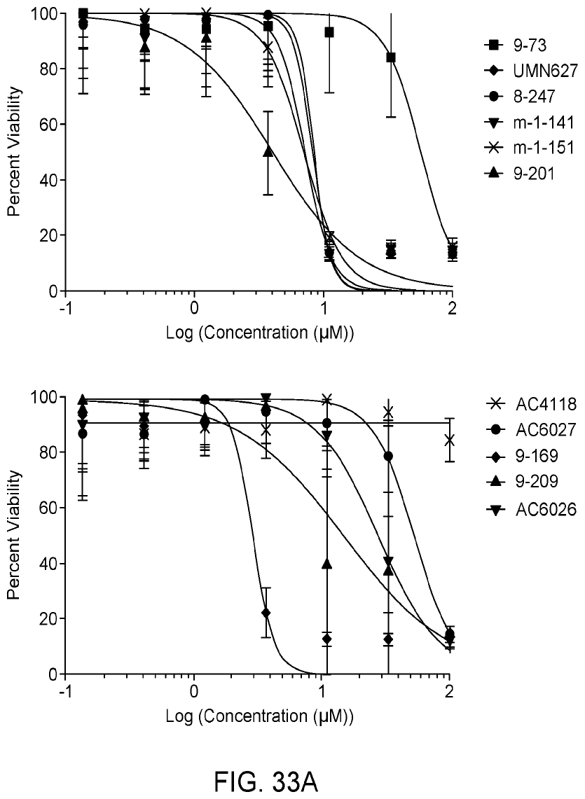

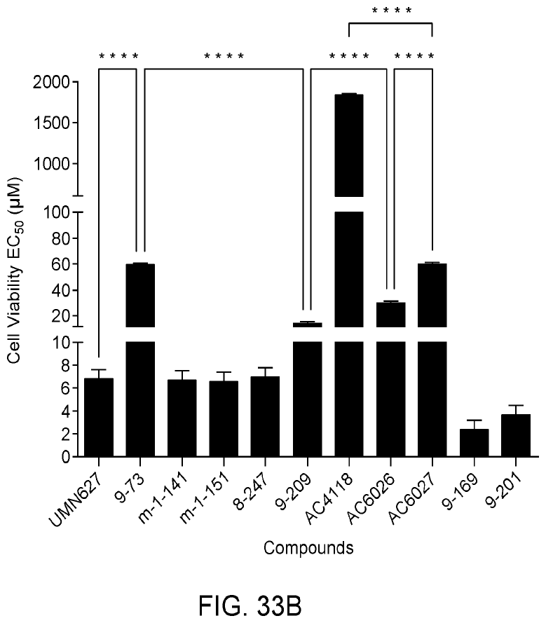

To get a better sense of the potential therapeutic window, the toxicity of the inhibitors was determined with a colorimetric cell viability assay (FIGS. 33A and 33B). The least toxic compounds were AC4118, AC6027, 9-73, AC6026, and 9-209 (FIG. 33B). On the other hand, 9-169 and 9-201 had markedly low EC₅₀ values below 4 μM. Based on the efficacy and toxicity, AC4118 and AC6027 were selected for in vivo models of inflammation to compare to previous measurements with UMN627.

### BRD4 Inhibitors Reduce Chemokine Expression in Preventative Inflammation Mouse Model

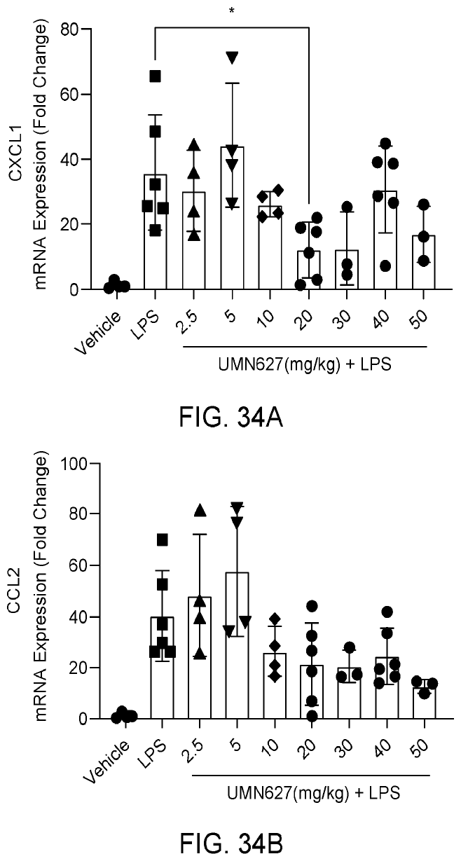

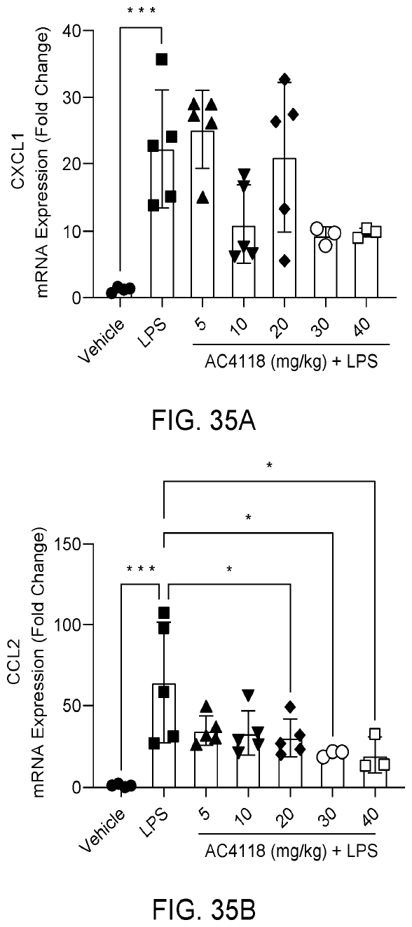

BRD4 inhibitors were introduced to murine models of mild liver inflammation induced by LPS. The BRD4 inhibitors acted as prophylactic agents to prevent the increased expression of CXCL1 and CCL2. UMN627 (FIGS. 34A and 34B) and AC4118 (FIGS. 35A and 35B) were given to mice at varying doses. The IC₅₀ for UMN627 was 10.9 mg drug per kg mouse (mg/kg) for CXCL1 and 8.8 mg/kg for CCL2. The IC₅₀ for AC4118 was lower <5 mg/kg. AC4118 was significantly reduced CCL2 expression at higher concentrations compared to UMN627.

### Methods

### Quantitative Real-Time Polymerase Chain Reaction

RNA extraction was performed with RNeasy kit (Qiagen 74104) from cells and mouse tissue according to the manufacturer's instructions. RNA Quantification was performed with spectrophotometry (NanoDrop, Thermo Scientific). 500 ng of mRNA was used for cDNA synthesis with dNTP and oligo primer using SuperScript™ III (Invitrogen 18080-093) for reverse transcription per the manufacturer's protocol. Real-Time PCR was performed from cDNA using IQ SYBR Green Mix (Biorad 1725121) on the 7500 Real-Time PCR system (Applied Biosystems), according to the manufacturer's instructions. Amplification of GAPDH and B-actin was performed for respective samples as internal controls. Each experiment was done in duplicates.

### Colorimetric Cell Viability Assay

Cell viability assay was performed with a XTT Viability Kit (Cell Signaling Technology, 9095) according to the manufacturer's instructions. HHSEC were seeded in three black-walled 96-well plates (Corning, 3603) with cell density≥12,000 cells per well.

The cells were incubated for 24 hours at 37° C., 5% CO₂ in endothelial growth media (Cell Application Inc, 211-500). After 24 hours, old media was removed and 100 μL of fresh media was added to each well. Drugs were dissolved in DMSO; a 1.1 mM stock solution was made by diluting drug in DPBS (11% DMSO, v/v). The 1.1 mM stock was serially diluted with 3-fold dilutions. DMSO volumes were balanced to 11% DMSO (v/v). A multichannel pipette was used to add 10 μL of drug or control stocks simultaneously (i.e., one column per drug per plate). The final maximum concentration of drug was 100 μM. Plates were incubated for 21 hours at 37° C., 5% CO₂. After incubation, 50 μL of XTT solution were added to each well and incubated for three hours to maximize differences in signal. Absorbance was measured at 462 and 620 nm with a spectrophotometer (Molecular Devices, Spectramax Plus 384). Background absorbance at 620 nm was subtracted from maximum peak at 462 nm.

### In Vitro LPS-Induced Inflammation Model

HHSEC were thawed and cultured according to standard cell culture conditions and according to manufacture instruction (ScienCell). Briefly, HHSEC are plated at low confluency and cultured in endothelial growth media (Cell, 211-500). Media was changed every other day and cells were split when close to confluency. Low passage cells (III-V) were plated at 70% confluency and cultured overnight. Serum starvation was performed by changing cells to low-serum medium (0.5% FBS in basal endothelial medium (Lonza CC-3121)) for 1.5 hours. After starvation, culture medium was changed into regular media without or with different concentration of BRD4 inhibitors for 2 hours. Then LPS (Invivogen tlrl-eblps) at a concentration of 200 ng/mL was added to the same culture media and incubated with cells for another 4 hours before cells were collected and assayed for downstream analysis.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for treating a mammal having a liver disease, wherein said method comprises administering a composition comprising an inhibitor of a BRD4 polypeptide to said mammal.

2. The method of claim 1, wherein said mammal is a human.

3. The method of claim 1, wherein said liver disease is an ALD.

4. The method of claim 3, wherein said ALD is alcoholic hepatitis.

5. The method of claim 1, wherein said method comprises identifying said mammal as having said liver disease.

6. The method of claim 1, wherein said method further comprises administering an agent used to treat a liver disease to said mammal.

7. The method of claim 6, wherein said agent is selected from the group consisting of nutritional supplements, corticosteroids, pentoxifylline, antibiotics, and a combination thereof.

8. The method of claim 1, wherein said method further comprises subjecting said mammal to a therapy used to treat a liver disease.

9. The method of claim 8, wherein said therapy is selected the group consisting of alcohol cessation counseling, liver transplantation, or alcohol cessation counseling and liver transplantation.

10. A method for reducing inflammation in a liver of a mammal having a liver disease, wherein said method comprises administering a composition comprising an inhibitor of a BRD4 polypeptide to said mammal.

11. The method of claim 10, wherein said mammal is a human.

12-13. (canceled)

14. A method for reducing a number of neutrophils in a liver of a mammal having a liver disease, wherein said method comprises administering a composition comprising an inhibitor of a BRD4 polypeptide to said mammal.

15. The method of claim 14, wherein said mammal is a human.

16-17. (canceled)

18. The method of claim 1, wherein said inhibitor is a compound of Formula (I): 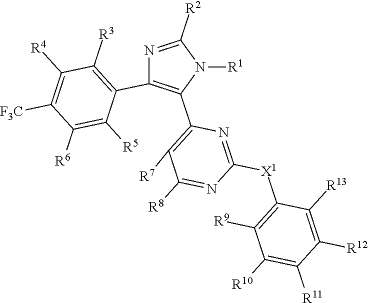
or a pharmaceutically acceptable salt thereof, wherein:
R¹ is a 4-7-membered heterocycloalkyl ring comprising at least one N atom, which is optionally substituted with 1, 2, or 3 R^{A} substituents independently selected from C₁₋₆ alkyl, C₂₋₆ alkenyl, C₂₋₆ alkynyl, C₁₋₄ haloalkyl, and C₃₋₅ cycloalkyl, each of which is optionally substituted with 1, 2, or 3 substituents independently selected from OH, NO₂, CN, amino, C₁₋₆ alkylamino, and di(C₁₋₆ alkyl)amino;
X¹ is selected from O and NR^{N};
R^{N} is selected from H, C₁₋₃ alkyl, and C₁₋₃ haloalkyl; and
each of R², R³, R⁴, R⁵, R⁶, R⁷, R⁸, R⁹, R¹⁰, R¹¹, R¹², and R¹³ is independently selected from H, OH, NO₂, CN, halo, C₁₋₆ alkyl, C₂₋₆ alkenyl, C₂₋₆ alkynyl, C₁₋₄ haloalkyl, C₁₋₆ alkoxy, and C₁₋₆ haloalkoxy, wherein each of said C₁₋₆ alkyl, C₂₋₆ alkenyl, and C₂₋₆ alkynyl is substituted with a substituent independently selected from OH, NO₂, CN, amino, C₁₋₆ alkylamino, and di(C₁₋₆ alkyl)amino.

19-23. (canceled)

24. The method of claim 18, wherein the compound of Formula (I) is selected from any one of the following compounds: 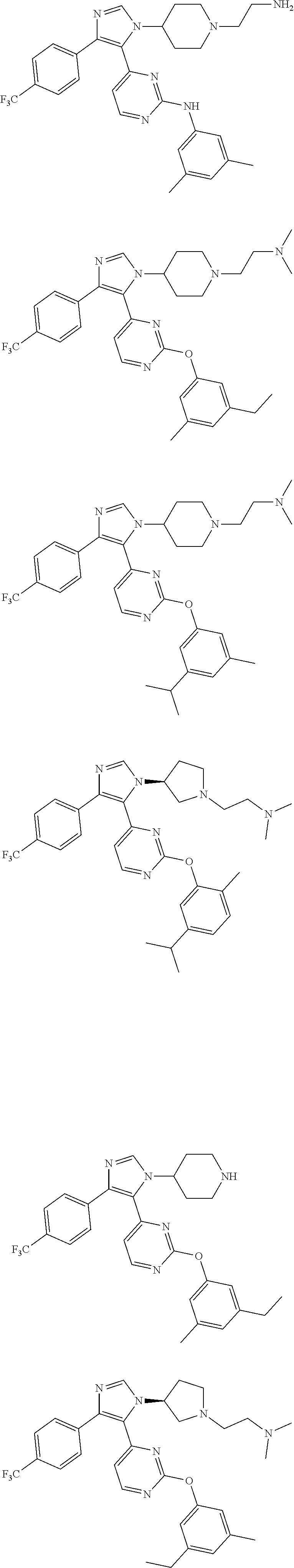 [SMILES: Cc1cc(C)cc(Nc2nccc(-c3c(-c4ccc(C(F)(F)F)cc4)ncn3C3CCN(CCN)CC3)n2)c1 ; CCc1cc(C)cc(Oc2nccc(-c3c(-c4ccc(C(F)(F)F)cc4)ncn3C3CCN(CCN(C)C)CC3)n2)c1 ; Cc1cc(Oc2nccc(-c3c(-c4ccc(C(F)(F)F)cc4)ncn3C3CCN(CCN(C)C)CC3)n2)cc(C(C)C)c1 ; Cc1ccc(C(C)C)cc1Oc1nccc(-c2c(-c3ccc(C(F)(F)F)cc3)ncn2[C@H]2CCN(CCN(C)C)C2)n1 ; CCc1cc(C)cc(Oc2nccc(-c3c(-c4ccc(C(F)(F)F)cc4)ncn3C3CCNCC3)n2)c1 ; CCc1cc(C)cc(Oc2nccc(-c3c(-c4ccc(C(F)(F)F)cc4)ncn3[C@H]3CCN(CCN(C)C)C3)n2)c1]
or a pharmaceutically acceptable salt thereof.

25. The method of claim 1, wherein said inhibitor is a compound of Formula (II): 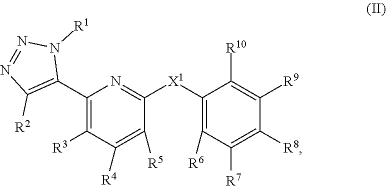
or a pharmaceutically acceptable salt thereof, wherein:
R¹ is a 4-7-membered heterocycloalkyl ring comprising at least one N atom, which is optionally substituted with 1, 2, or 3 RA substituents independently selected from C₁₋₆ alkyl, C₂₋₆ alkenyl, C₂₋₆ alkynyl, C₁₋₄ haloalkyl, and C₃₋₅ cycloalkyl, each of which is optionally substituted with 1, 2, or 3 substituents independently selected from OH, NO₂, CN, amino, C₁₋₆ alkylamino, and di(C₁₋₆ alkyl)amino;
X¹ is selected from O and NR^{N};
R^{N} is selected from H, C₁₋₃ alkyl, and C₁₋₃ haloalkyl; and
each of R², R³, R⁴, R⁵, R⁶, R⁷, R⁸, R⁹, and R¹⁰ is independently selected from H, OH, NO₂, CN, halo, C₁₋₆ alkyl, C₂₋₆ alkenyl, C₂₋₆ alkynyl, C₁₋₄ haloalkyl, C₁₋₆ alkoxy, and C₁₋₆ haloalkoxy, wherein each of said C₁₋₆ alkyl, C₂₋₆ alkenyl, and C₂₋₆ alkynyl is substituted with a substituent independently selected from OH, NO₂, CN, amino, C₁₋₆ alkylamino, and di(C₁₋₆ alkyl)amino.

26-27. (canceled)

28. The composition of claim 25, wherein the compound of Formula (II) is selected from any one of the following compounds: 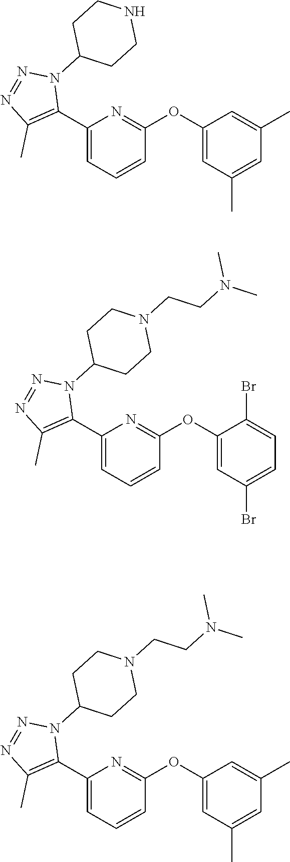
or a pharmaceutically acceptable salt thereof.

29. The method of claim 1, wherein said inhibitor is a compound of Formula (III): 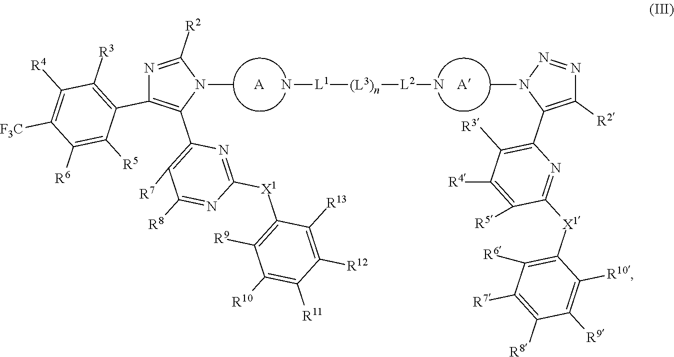
or a pharmaceutically acceptable salt thereof, wherein:
each of ring A and ring A′ is independently a 4-7-membered heterocycloalkyl ring comprising at least one N atom, which is optionally substituted with 1, 2, or 3 RA substituents independently selected from C₁₋₆ alkyl, C₂₋₆ alkenyl, C₂₋₆ alkynyl, C₁₋₄ haloalkyl, and C₃₋₅ cycloalkyl, each of which is optionally substituted with 1, 2, or 3 substituents independently selected from OH, NO₂, CN, amino, C₁₋₆ alkylamino, and di(C₁₋₆ alkyl)amino;
each of X¹ and X¹′ is independently selected from O and NR^{N};
each R^{N} is independently selected from H, C₁₋₃ alkyl, and C₁₋₃ haloalkyl;
each of R², R³, R⁴, R⁵, R⁶, R⁷, R⁸, R⁹, R¹⁰, R¹¹, R¹², R¹³, R^{2′}, R^{3′}, R^{4′}, R^{5′}, R^{6′}, R^{7′}, R^{8′}, R^{9′}, and R^{10′} is independently selected from H, OH, NO₂, CN, halo, C₁₋₆ alkyl, C₂₋₆ alkenyl, C₂₋₆ alkynyl, C₁₋₄ haloalkyl, C₁₋₆ alkoxy, and C₁₋₆ haloalkoxy, wherein each of said C₁₋₆ alkyl, C₂₋₆ alkenyl, and C₂₋₆ alkynyl is substituted with a substituent independently selected from OH, NO₂, CN, amino, C₁₋₆ alkylamino, and di(C₁₋₆ alkyl)amino;
each of L¹ and L² is independently C₁₋₃ alkylene, optionally substituted with 1, 2, or 3 substituents independently selected from OH, NO₂, CN, halo, amino, and carboxy;
n is an integer selected from 1 to 10; and
each L³ is independently selected from C(═O), N(R^{N}), O, (—C₁₋₃ alkylene-O—)ₓ, (—O—C₁₋₃ alkylene-)ₓ, and —C₁₋₃ alkylene-, wherein each x is independently an integer from 1 to 10 and each C₁₋₃ alkylene is optionally substituted with 1, 2, or 3 substituents independently selected from OH, NO₂, CN, halo, amino, and carboxy.

30. (canceled)

31. The composition of claim 29, wherein the compound of Formula (III) is selected from any one of the following compounds: 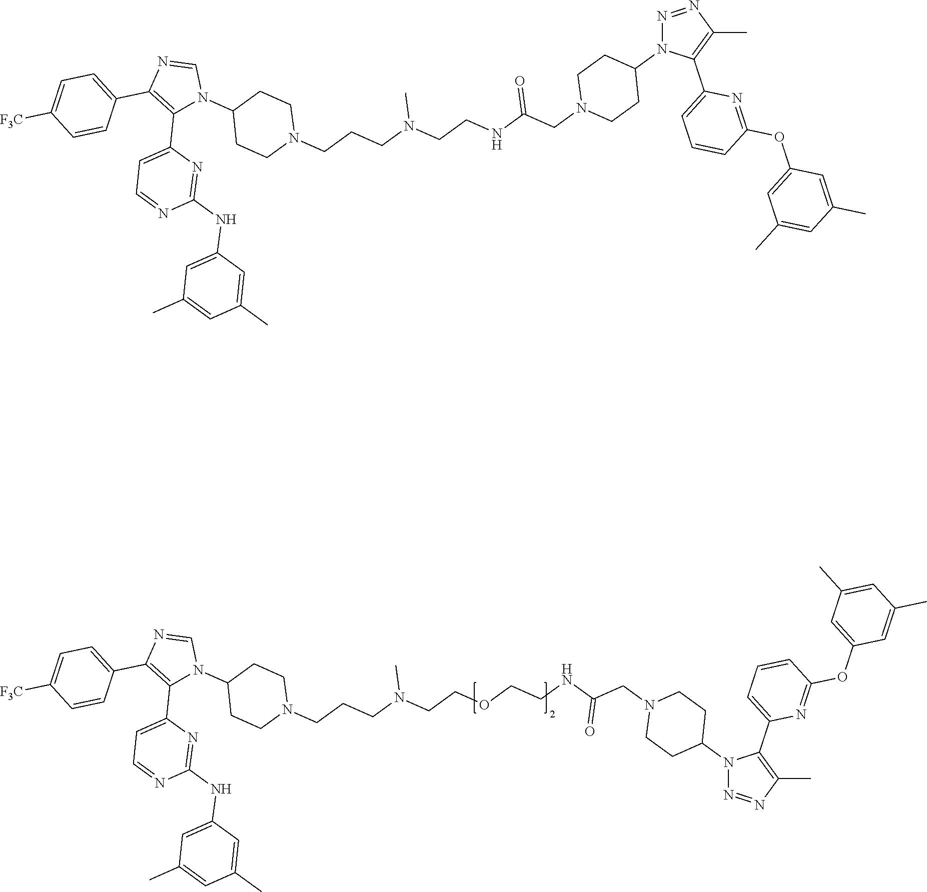
or a pharmaceutically acceptable salt thereof.

32-34. (canceled)
